(19) United States
(12) Patent Application Publication
(10) Pub. No.: US 2003/0215456 A1
(43) Pub. Date: Nov. 20, 2003

(54) Method of treating cancer

(76) Inventors: Sui-Long Yao, West Windsor, NJ (US); Raymond E. Jones, Lansdale, PA (US); Deborah DeFeo-Jones, Lansdale, PA (US); David C. Heimbrook, Coopersburg, PA (US); Patricia Rhymer, Martinsville, NJ (US); Pamela J. Wasserbly, Doylestown, PA (US)

Correspondence Address: MERCK AND CO INC, P O BOX 2000, RAHWAY, NJ, 070650907

(21) Appl. No.: 09/969,322

(22) Filed: Oct. 2, 2001

(51) Int. Cl. A61K 39/00; A61K 38/14

(52) U.S. Cl. 424/185.1; 424/277.1; 514/8

(57) ABSTRACT

The present invention relates to methods of treating cancer using a combination of a compound which is a PSA conjugate and a tachykinin receptor antagonist, which methods comprise administering to said mammal, either sequentially in any order or simultaneously, amounts of at least two therapeutic agents selected from a group consisting of a compound which is a PSA conjugate and a tachykinin receptor antagonist. The invention also relates to methods of preparing such compositions.

### BACKGROUND OF THE INVENTION

The present invention relates to methods of treating cancer, and more particularly cancer associated with cells that produce prostate specific antigen (PSA), which comprise administering to a patient in need thereof at least one tachykinin receptor antagonist, in particular a neurokinin-1 (NK-1) receptor antagonist, and at least one conjugate, which comprises an oligopeptide that is selectively cleaved by PSA and a cytotoxic agent.

In 1999, new cases of cancer of the prostate gland were expected to be diagnosed in 179,300 men in the U.S. and 37,000 American males were expected to die from this disease (Landis, S. H. et al. CA Cancer J. Clin. 49:8-31 (1999)). Prostate cancer is the most frequently diagnosed malignancy (other than that of the skin) in U.S. men and the second leading cause of cancer-related deaths (behind lung cancer) in that group. In 2000, the Surveillance Research Program of the American Cancer Society reported its findings of the estimated cancer incidence, mortality and survival data in the United States. According to the report, the total number of cancer deaths among men had decreased for the first time in 70 years. This decrease, which occurred from 1996 to 1997 is attributed to the recent down-turns in lung and bronchus cancer deaths, prostate cancer deaths and colon and rectum cancer deaths. (R. T. Greenlee et al., CA Cancer J. Clin. (2000) 50(1):7-33).

Prostate specific Antigen (PSA) is a single chain 33 kDa glycoprotein that is produced almost exclusively by the human prostate epithelium and occurs at levels of 0.5 to 4.0 ng/ml in human seminal fluid (Nadji, M., Taber, S. Z., Castro, A., et al. (1981) Cancer 48:1229; Papsidero, L., Kuriyama, M., Wang, M., et al. (1981), JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979), Invest. Urol. 17:159; W. J. Catalona et al., New Engl. J. Med. (1991) 324(17):1156-61). The single carbo-hydrate unit is attached at asparagine residue number 45 and accounts for 2 to 3 kDa of the total molecular mass. PSA is a protease with chymotrypsin-like specificity (Christensson, A., Laurell, C. B., Lilja, H. (1990), Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985), J. Clin. Invest. 76:1899; Lilja, H., Oldbring, J., Rannevik, G., et al. (1987), J. Clin. Invest. 80:281; McGee, R. S., Herr, J. C. (1988), Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984), Scand. J. Clin. Lab. Invest. 44:447; McGee, R. S., Herr, J. C. (1987), Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Björk, T., Nilsson, O., et al. (1993), J. Urol. 150: 100-105; Lilja, H., Christensson, A., Dahlén, U. (1991), Clin. Chem. 37:1618-1625; Stenman, U. H., Leinoven, J., Alfthan, H., et al. (1991), Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991), Biochemistry 30:1723-1730; Perlmutter, D. H., Glover, G. I., Rivetna, M., et al. (1990), Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes aminor fraction of the serum PSA (Christensson, A., Björk, T., Nilsson, O., et al. (1993), J. Urol. 150:100-105; Lilja, H., Christensson, A., Dahlén, U. (1991), Clin. Chem. 37:1618-1625). The size of this form of serum PSA is similar to that of PSA in seminal fluid (Lilja, H., Christensson, A., Dahlén, U. (1991), Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Björk, T., Nilsson, O., et al. (1993), J. Urol. 150:100-105; Lilja, H., Christensson, A., Dahlén, U. (1991), Clin. Chem. 37:1618-1625).

Serum measurements of PSA are useful for monitoring the treatment of adenocarcinoma of the prostate (Duffy, M. S. (1989), Ann. Clin. Biochem. 26:379-387; Brawer, M. K. and Lange, P. H. (1989), Urol. Suppl. 5:11-16; Hara, M. and Kimura, H. (1989), J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991), Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985), Brit. J. Urology 57:50-55). Therefore, a cytotoxic compound that could be activated by the proteolytic activity of PSA should be prostate cell specific as well as specific for PSA secreting prostate metastases.

Conjugates which comprise an oligopeptide which can be selectively cleaved by enzymatically active PSA attached, either directly or via a linker to a cytotoxic agent and which are useful in the treatment of prostate cancer and benign prostatic hyperplasia have been previously described (U.S. Pat. Nos. 5,599,686 and 5,866,679).

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to SP the known mammalian tachykinins include neurokinin A and neurokinin B. The current nomenclature designates the receptors for substance P, neurokinin A, and neurokinin B as neurokinin-1, neurokinin-2, and neurokinin-3, respectively.

The neuropeptide receptors for substance P (neurokinin-1; NK-1) are widely distributed throughout the mammalian nervous system (especially brain and spinal ganglia), the circulatory system and peripheral tissues (especially the duodenum and jejunum) and are involved in regulating a number of diverse biological processes. This includes sensory perception of olfaction, vision, audition and pain, movement control, gastric motility, vasodilation, salivation, and micturition (B. Pernow, Pharmacol. Rev., 1983, 35, 85-141).

Substance P is a pharmacologically-active neuropeptide that is produced in mammals and acts as a vasodilator, a depressant, stimulates salivation and produces increased capillary permeability. It is also capable of producing both analgesia and hyperalgesia in animals, depending on dose and pain responsiveness of the animal (see R. C. A. Frederickson et al., Science, 199, 1359 (1978); P. Oehme et al., Science, 208, 305 (1980)) and plays a role in sensory transmission and pain perception (T. M. Jessell, Advan. Biochem. Psychopharmacol. 28, 189 (1981)).

Conditions in which substance P has been implicated include disorders of bladder function, such as cystitis, bladder detrusor hyperreflexia, and urinary incontinence (see PCT International Patent Publication No. WO 95/16679 and EPO Patent Publication No. EP 0,610,021). It has been suggested that substance P is implicated in certain urinary tract conditions (Chapple C R, et al., Journal of Urology, 146:1637-1644 (1991); Danuser H, et al., Journal of Urology, 157:1018-1024 (1997); Palea S, et al., Journal of Pharmacology and Experimental Therapeutics, 277:700-705 (1996); Tainio H, Acta. Histochem., 97:113-119 (1995)). It has also been suggested that neurokinin-2 receptor antagonists might be useful for treatment of urinary bladder disorders and interstitial cystitis (Palea S, et al., “Pharmacological characterization of tachykinin NK₂ receptors on isolated human urinary bladder, prostatic urethra, and prostate,” Journal of Pharmacology and Experimental Therapeutics, 277:700-705 (1996)).

It is the object of the instant invention to provide a method for treating cancer, and more particularly cancer associated with cells that produce prostate specific antigen (PSA), which offers advantages over previously disclosed methods of treatment.

### SUMMARY OF THE INVENTION

A method of treating cancer, and more particularly cancer associated with cells that produce prostate specific antigen (PSA), is disclosed which is comprised of administering to a patient in need of such treatment amounts of at least one tachykinin receptor antagonist, in particular a neurokinin-1 (NK-1) receptor antagonist, and at least one conjugate, which comprises an oligopeptide that is selectively cleaved by PSA and a cytotoxic agent.

### DETAILED DESCRIPTION OF THE INVENTION

The present invention relates to a method of treating cancer, and more particularly cancer associated with cells that produce and secrete prostate specific antigen (PSA), which is comprised of administering to a patient in need of such treatment amounts of at least one tachykinin receptor antagonist, in particular a neurokinin-1 (NK-1) receptor antagonist, and at least one conjugate, which comprises an oligopeptide that is selectively cleaved by PSA and a cytotoxic agent (hereinafter referred to as a PSA conjugate).

In practicing the instant method of treatment, it is understood that the neurokinin-1 (NK-1) receptor antagonist and the PSA conjugate(s) may be administered either simultaneously in a single pharmaceutical composition or individually in separate pharmaceutical compositions. If the neurokinin-1 (NK-1) receptor antagonist and the PSA conjugate(s) are administered in separate compositions, such compositions may be administered simultaneously or consecutively.

The term “consecutively” when used in the context of administration of two or more separate pharmaceutical compositions means that administrations of the separate pharmaceutical compositions are at separate times. The term “consecutively” also includes administration of two or more separate pharmaceutical compositions wherein administration of one or more pharmaceutical compositions is a continuous administration over a prolonged period of time and wherein administration of another of the compositions occur at a discrete time during the prolonged period.

The PSA conjugate administered in the instant invention comprises an oligopeptide, which is specifically recognized by the free prostate specific antigen (PSA) and is capable of being proteolytically cleaved by the enzymatic activity of the free prostate specific antigen. This oligopeptide may be covalently bonded directly, or through a chemical linker, to a cytotoxic agent. Ideally, the cytotoxic activity of the cytotoxic agent is greatly reduced or absent when the oligopeptide containing the PSA proteolytic cleavage site is bonded directly, or through a chemical linker, to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site. While it is not necessary for practicing this aspect of the invention, a preferred embodiment of this aspect of the invention is a conjugate wherein the oligopeptide, and the chemical linker if present, are detached from the cytotoxic agent by the proteolytic activity of the free PSA and any other native proteolytic enzymes present in the tissue proximity, thereby releasing unmodified cytotoxic agent into the physiological environment at the place of proteolytic cleavage. Pharmaceutically acceptable salts of the conjugates are also included.

PSA conjugates, which contain oligopeptides that are selectively cleaved by enzymatically active PSA, can be identified by a number of assays, in particularly the assays described in Examples 24-28.

In one embodiment of the instant invention, the oligopeptide component of the PSA conjugate incorporates a cyclic amino acid having a hydrophilic substituent as part of the oligopeptides, said cyclic amino acid contributes to the aqueous solubility of the conjugate. Examples of such hydrophilic cyclic amino acids include but are not limited to hydroxylated, polyhydroxylated and alkoxylated proline and pipecolic acid moieties.

In a preferred embodiment of the invention, the oligopeptide component of the PSA conjugate is characterized by having a protecting group on the terminus amino acid moiety that is not attached to the cytotoxic agent. Such protection of the terminal amino acid reduces or eliminates the enzymatic degradation of such peptidyl therapeutic agents by the action of exogenous aminopeptidases and carboxypeptidases which are present in the blood plasma of warm blooded animals. Examples of protecting groups that may be attached to the amino moiety of an N-terminus oligopeptide include, but are not limited to acetyl, benzoyl, pivaloyl, succinyl, glutaryl, hydoxyalkanoyl, polyhydroxyalkanoyl, polyethylene glycol (PEG) containing alkanoyl and the like. Examples of protecting groups that may be attached to the carboxylic acid of a C-terminus oligopeptide include, but are not limited to, formation of an organic or inorganic ester of the carboxylic acid, such as an alkyl, aralkyl, aryl, polyether ester, phosphoryl and sulfuryl, or conversion of the carboxylic acid moiety to a substituted or unsubstituted amide moiety. The N-terminus or C-terminus of the oligopeptide may also be substituted with a unnatural amino acid, such as β-alanine, or a D-amino acid, such as a D-valyl or D-alanyl group.

It is understood that the oligopeptide which is conjugated to the cytotoxic agent, whether through a direct covalent bond or through a chemical linker, does not need to be the oligopeptide that has the greatest recognition by free PSA and is most readily proteolytically cleaved by free PSA. Thus, the oligopeptide that is selected for incorporation in such conjugate will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage.

Because the PSA conjugates useful in the instant compositions can be used for modifying a given biological response, the cytotoxic agent component of the PSA conjugate is not to be construed as limited to classical chemical therapeutic agents. For example, the cytotoxic agent may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, b-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The preferred cytotoxic agents include, in general, alkylating agents, antiproliferative agents, tubulin binding agents and the like. Preferred classes of cytotoxic agents include, for example, the anthracycline family of drugs, the vinca drugs, the taxanes, the mitomycins, the bleomycins, the cytotoxic nucleosides, the pteridine family of drugs, diynenes, and the podophyllotoxins. Particularly useful members of those classes include, for example, doxorubicin, caminomycin, daunorubicin, aminopterin, methotrexate, methopterin, dichloro-methotrexate, mitomycin C, porfiromycin, paclitaxel, docetaxel, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. Other useful cytotoxic agents include estramustine, cisplatin and cyclophosphamide. One skilled in the art may make chemical modifications to the desired cytotoxic agent in order to make reactions of that compound more convenient for purposes of preparing PSA conjugates of the invention.

Preferably the cytotoxic agent component of the PSA conjugate is selected from a member of a class of cytotoxic agents selected from the vinca alkaloid drugs and the anthracyclines.

PSA conjugates that are useful in the methods of the instant invention and are identified by the properties described hereinabove include:

a) a compound represented by the formula I:

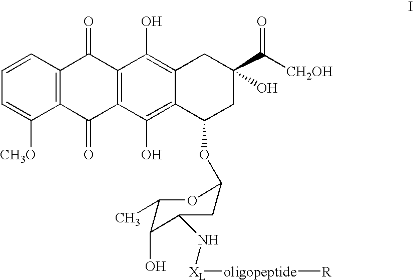

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine;

R is hydrogen or —(C═O)R¹; and

R¹ is C₁-C₆-alkyl or aryl,

or the pharmaceutically acceptable salt thereof;

b) a compound represented by the formula II:

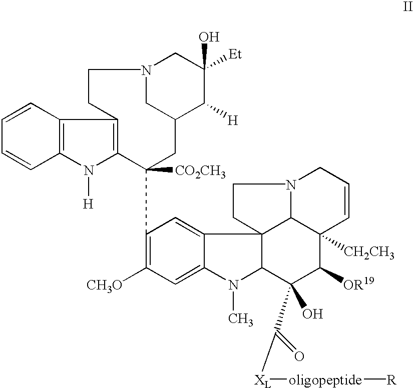

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine; or

X_{L} is —NH—(CH₂)ₙ—NH—;

R is hydrogen or —(C═O)R¹;

R¹ is C₁-C₆-alkyl or aryl;

R¹⁹ is hydrogen or acetyl; and

n is 1, 2, 3, 4 or 5,

or the pharmaceutically acceptable salt thereof;

c) a compound represented by the formula III:

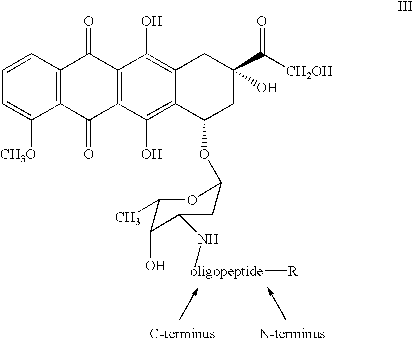

wherein:

oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula:

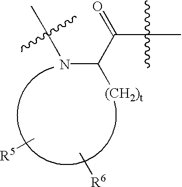

and wherein

the C-terminus carbonyl is covalently bound to the amine of doxorubicin;

R is selected from

a) hydrogen,

b) —(C═O)R^{1a},

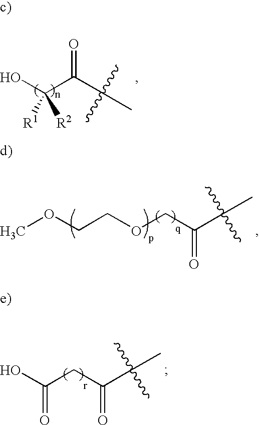

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

n is 1, 2, 3 or 4;

p is zero or an integer between 1 and 100;

q is 0 or 1, provided that if p is zero, q is 1;

r is an integer between 1 and 10; and

t is 3 or 4;

or a pharmaceutically acceptable salt thereof;

d) a compound represented by the formula IV:

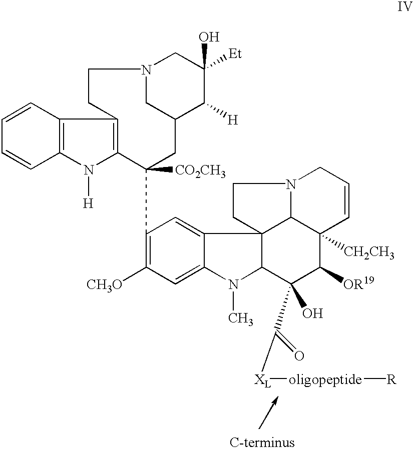

wherein:

oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula:

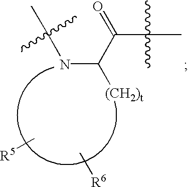

X_{L} is —NH—(CH₂)ᵤ—NH—;

R is selected from

a) hydrogen,

b) (C═O)R^{1a},

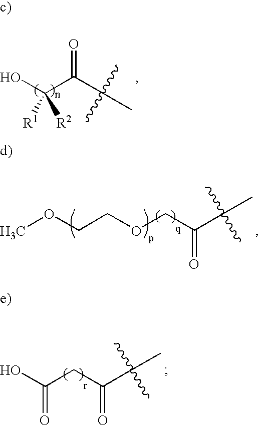

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2 or 3;

t is 3 or 4;

u is 1, 2, 3, 4 or 5;

or the pharmaceutically acceptable salt thereof;

e) a compound represented by the formula V:

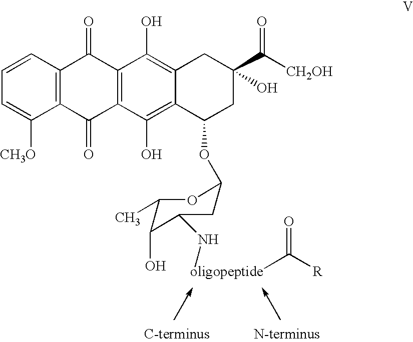

wherein:

oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;

R is selected from

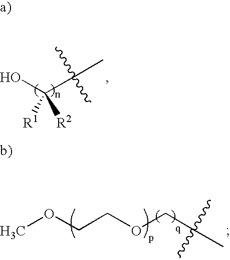

R¹ and R² are independently selected from: hydrogen, OH, C₁-C₆ alkyl, C₁-C₆ alkoxy, C₁-C₆ aralkyl and aryl;

n is 1, 2, 3 or 4;

p is zero or an integer between 1 and 100;

q is 0 or 1, provided that if p is zero, q is 1;

or the pharmaceutically acceptable salt thereof;

f) a compound represented by the formula VI:

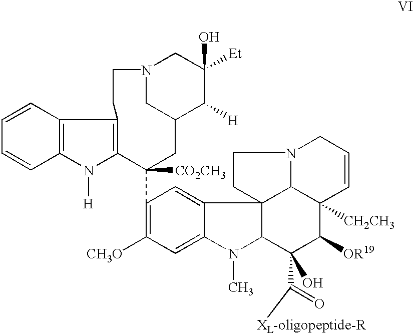

wherein:

oligopeptide is an oligopeptide which is specifically recognized by the free prostate specific antigen (PSA) and is capable of being proteolytically cleaved by the enzymatic activity of the free prostate specific antigen;

X_{L} is —NH—(CH₂)ᵣNH—;

R is selected from

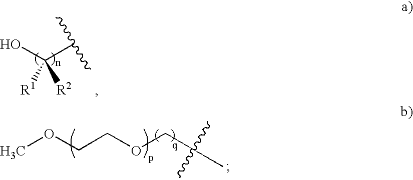

R¹ and R² are independently selected from: hydrogen, OH, C₁-C₆ alkyl, C₁-C₆ alkoxy, C₁-C₆ aralkyl and aryl;

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is O or 1, provided that if p is zero, q is 1;

r is 1, 2, 3, 4 or 5;

or the pharmaceutically acceptable salt thereof;

g) a compound represented by the formula VII:

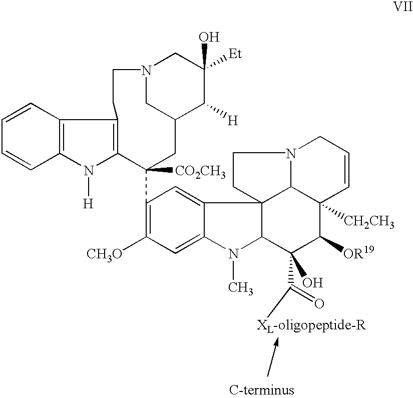

wherein:

oligopeptide is an oligopeptide which is specifically recognized by the free prostate specific antigen (PSA) and is capable of being proteolytically cleaved by the enzymatic activity of the free prostate specific antigen,

X_{L} is —NH—(CH₂)ᵤ—W—(CH₂)ᵤ—NH—;

R is selected from

a) hydrogen,

b) —(C═O)R^{1a},

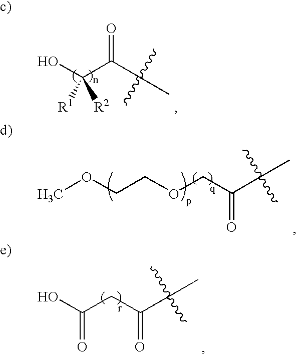

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₆-cycloalkyl, hydroxylated aryl, polyhydroxylated aryl or aryl;

W is selected from cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt thereof; and

h) a compound represented by the formula VIII:

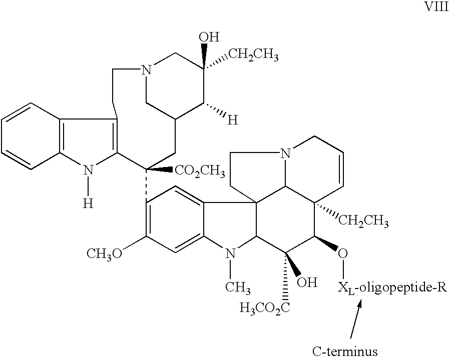

wherein:

oligopeptide is an oligopeptide which is specifically recognized by the free prostate specific antigen (PSA) and is capable of being proteolytically cleaved by the enzymatic activity of the free prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from

a) hydrogen,

b) —(C═O)R^{1a},

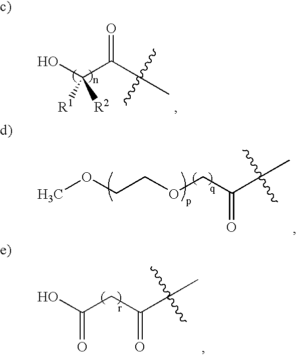

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

W is selected from a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt or optical isomer thereof.

Examples of compounds which are PSA conjugates include the following:

| | |
|---|---|
| i) | |
| 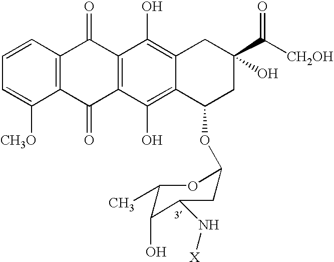 | |
| wherein X is: | |
| AsnLysIleSerTyrGlnSer- | (SEQ.ID.NO.: 1), |
| AsnLysIleSerTyrGlnSerSer- | (SEQ.ID.NO.: 2), |
| AsnLysIleSerTyrGlnSerSerSer- | (SEQ.ID.NO.: 3), |
| AsnLysIleSerTyrGlnSerSerSerThr- | (SEQ.ID.NO.: 4), |
| AsnLysIleSerTyrGlnSerSerSerThrGlu- | (SEQ.ID.NO.: 5), |
| AlaAsnLysIleSerTyrGlnSerSerSerThrGlu- | (SEQ.ID.NO.: 6), |
| Ac-AlaAsnLysIleSerTyrGlnSerSerSerThr- | (SEQ.ID.NO.: 7), |
| Ac-AlaAsnLysIleSerTyrGlnSerSerSerThrLeu- | (SEQ.ID.NO.: 8), |
| Ac-AlaAsnLysAlaSerTyrGlnSerAlaSerThrLeu- | (SEQ.ID.NO.: 9), |
| Ac-AlaAsnLysAlaSerTyrGlnSerAlaSerLeu- | (SEQ.ID.NO.: 10), |
| Ac-AlaAsnLysAlaSerTyrGlnSerSerSerLeu- | (SEQ.ID.NO.: 11), |
| Ac-AlaAsnLysAlaSerTyrGlnSerSerLeu- | (SEQ.ID.NO.: 12), |
| Ac-SerTyrGlnSerSerSerLeu- | (SEQ.ID.NO.: 13), |
| Ac-hArgTyrGlnSerSerSerLeu- | (SEQ.ID.NO.: 14). |
| Ac-LysTyrGlnSerSerSerLeu- | (SEQ.ID.NO.: 15), or |
| Ac-LysTyrGinSerSerNle- | (SEQ.ID.NO.: 16); |
| ii) | |
| 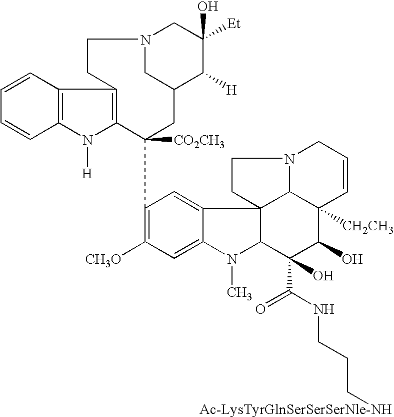 | (SEQ.ID.NO.: 17), or |
| 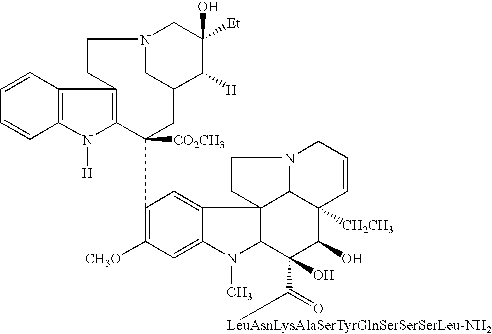 | (SEQ.ID.NO.: 18); |
| iii) | |
| 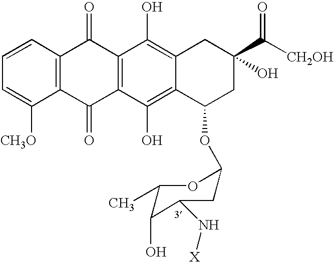 | |
| wherein X is: | |
| 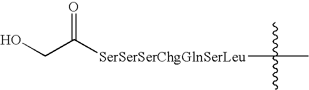 | (SEQ.ID.NO.: 19), |
| 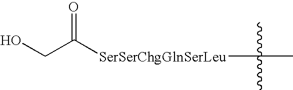 | (SEQ.ID.NO.: 20), or |
| 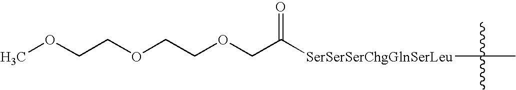 | (SEQ.ID.NO.: 21); |
| iv) | |
| 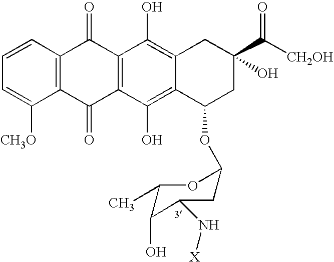 | |
| wherein X is: | |
| 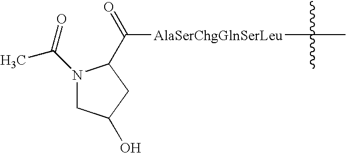 | (SEQ.ID.NO.: 22), |
| 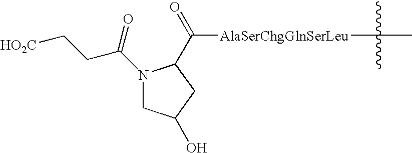 | (SEQ.ID.NO.: 23), |
| 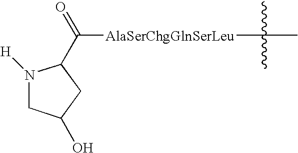 | (SEQ.ID.NO.: 24), |
| 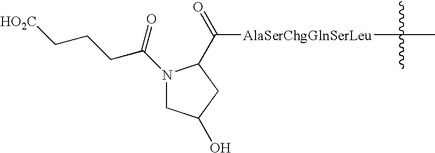 | (SEQ.ID.NO.: 25), |
| 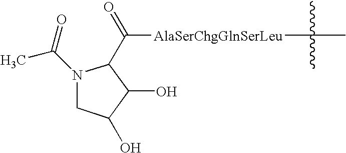 | (SEQ.ID.NO.: 26), |
| 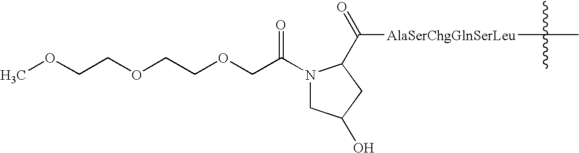 | (SEQ.ID.NO.: 27), or |
| 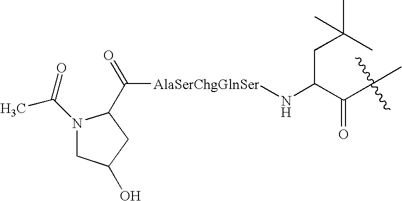 | (SEQ.ID.NO.: 28); |
| v) | |
| 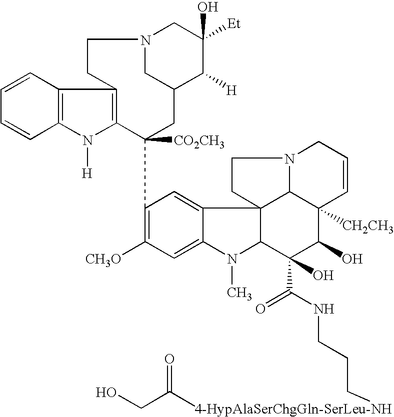 | (SEQ.ID.NO.: 29), |
| 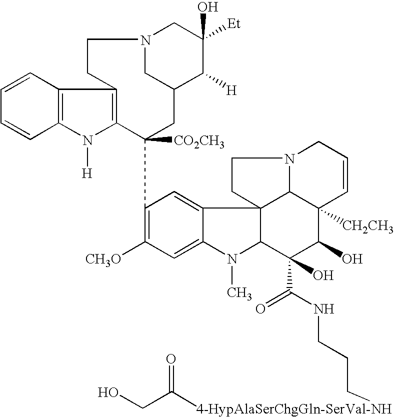 | (SEQ.ID.NO.: 30), or |
| 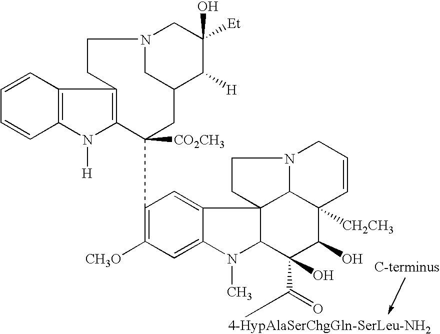 | (SEQ.ID.NO. 31); |
| vi) | |
| 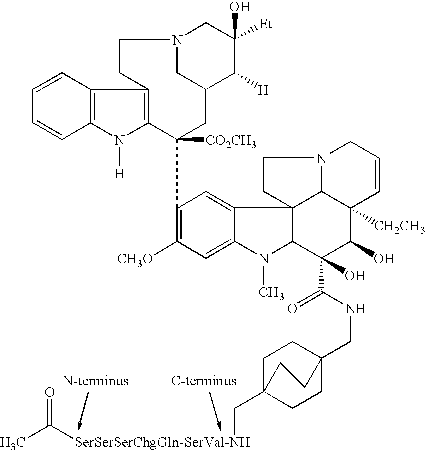 | (SEQ.ID.NO.: 32), |
| 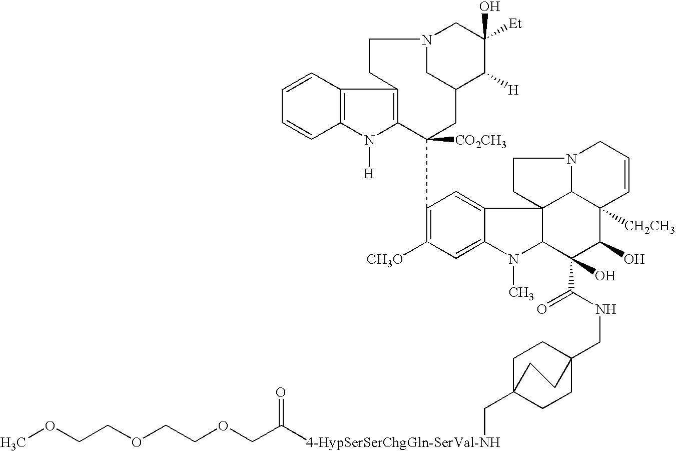 | (SEQ.ID.NO.: 33), |
| 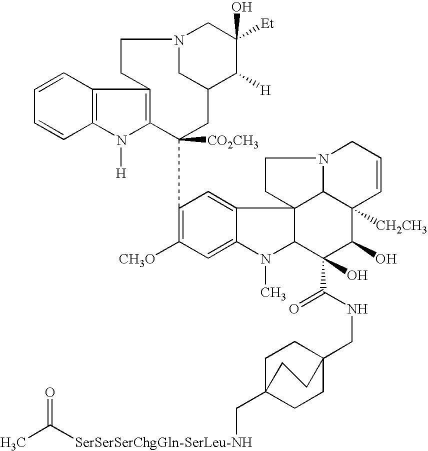 | (SEQ.ID.NO.: 34), or |
| 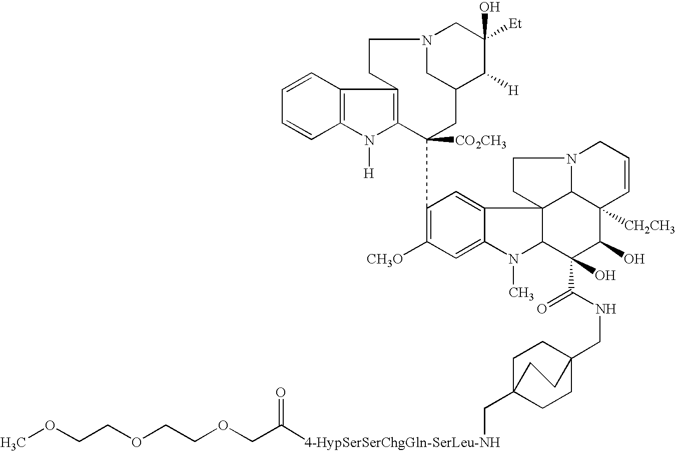 | (SEQ.ID.NO.: 35); |
| vii) | |
| 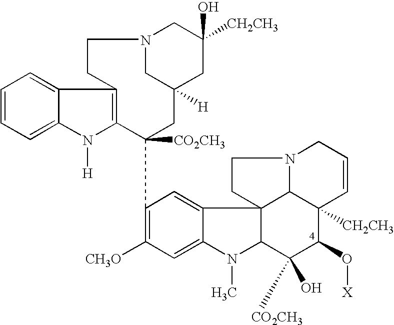 | |
| wherein X is | |
| 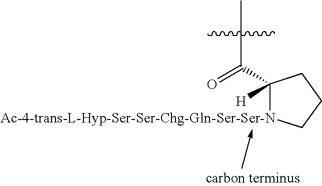 | (SEQ.ID.NO.: 36), |
| 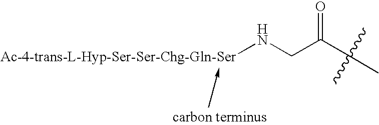 | (SEQ.ID.NO.: 37), |
| 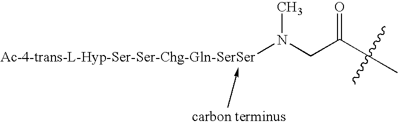 | (SEQ.ID.NO.: 38), |
| 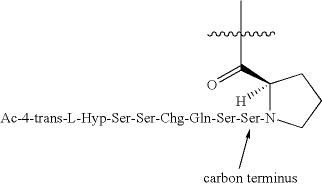 | (SEQ.ID.NO.: 39), |
| 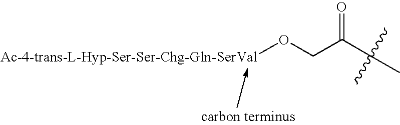 | (SEQ.ID.NO.: 40), |
| 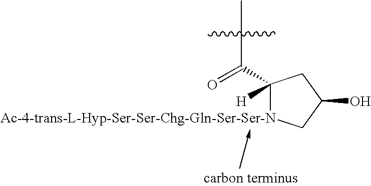 | (SEQ.ID.NO.: 41), |
| 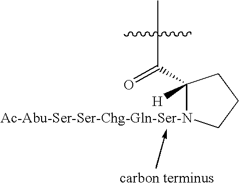 | (SEQ.ID.NO.: 42), |
| 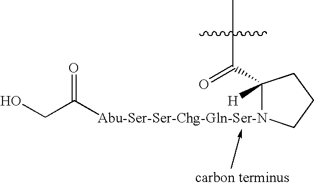 | (SEQ.ID.NO.: 43), |
| 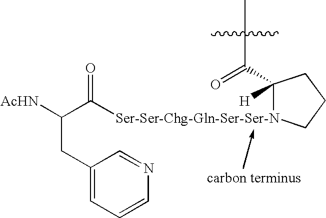 | (SEQ.ID.NO.: 44), |
| 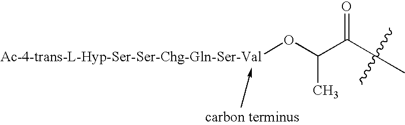 | (SEQ.ID.NO.: 45), or |
| 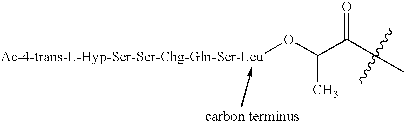 | (SEQ.ID.NO.: 46); |
| or the pharmaceutically acceptable salt or optical isomer thereof. | |
| Preferably the method of the instant invention comprises the PSA | |
| conjugate | |
| 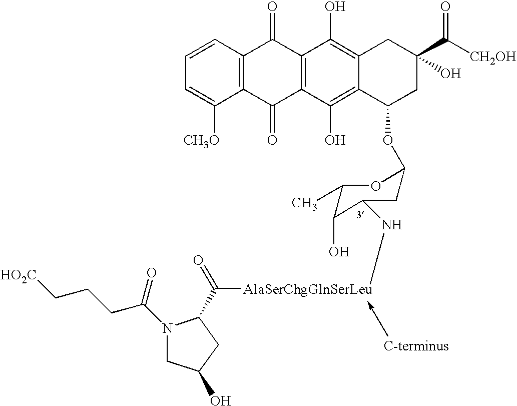 | (SEQ.ID.NO.: 25), |
| or the pharmaceutically acceptable salt thereof. | |

Compounds which are PSA conjugates and are therefore useful in the present invention, and methods of synthesis thereof, can be found in the following patents, pending applications and publications, which are herein incorporated by reference:

U.S. Pat. No. 5,599,686 granted on Feb. 4, 1997;

WO 96/00503 (Jan. 11, 1996); U.S. Ser. No. 08/404,833 filed on Mar. 15, 1995;

U.S. Ser. No. 08/468,161 filed on Jun. 6, 1995;

U.S. Pat. No. 5,866,679 granted on Feb. 2, 1999;

WO 98/10651 (Mar. 19, 1998); U.S. Ser. No. 08/926,412 filed on Sep. 9, 1997;

WO 98/18493 (May 7, 1998); U.S. Pat. No. 5,948,750, granted on Sep. 7, 1999

U.S. Ser. No. 09/112,656 filed on Jul. 9, 1998; U.S. S No. 60/052,195 filed on Jul. 10, 1997; and

U.S. Ser. No. 09/193,365 filed on Nov. 17, 1998; U.S. S No. 60/067,110 filed on Dec. 2, 1997.

Compounds which are described as prodrugs wherein the active therapeutic agent is release by the action of enzymatically active PSA and therefore may be useful in the present invention, and methods of synthesis thereof, can be found in the following patents, pending applications and publications, which are herein incorporated by reference: WO 98/52966 (Nov. 26, 1998).

All patents, publications and pending patent applications identified are hereby incorporated by reference.

With respect to the compounds of formulas I through VIII the following definitions apply:

“Alkyl” means linear branched and cyclic structures, and combinations thereof, containing the indicated number of carbon atoms. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, cyclopropyl, cyclopentyl, cycloheptyl, adamantyl, cyclododecylmethyl, 2-ethyl-1-bicyclo[4.4.0]decyl and the like.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Halogen” or “halo” as used herein means fluoro, chloro, bromo and iodo.

“Fluoro alkyl” means alkyl groups in which one or more hydrogen is replaced by fluorine. Examples are —CF₃, —CH₂CH₂F, —CH₂CF₃, c-Pr-F₅, c-Hex-F₁₁ and the like. Similarly, fluoroalkoxy means linear, branched and cyclic structures, with the indicated number of carbon atoms.

For purposes of this specification “Alkoxy” means alkoxy groups of the indicated number of carbon atoms of a straight, branched, or cyclic configuration. Examples of alkoxy groups include methoxy, ethoxy, propoxy, isopropoxy, cyclopropyloxy, cyclohexyloxy, and the like.

“Alkylthio” means alkylthio groups of the indicated number of carbon atoms of a straight, branched or cyclic configuration. Examples of alkylthio groups include methylthio, propylthio, isopropylthio, cycloheptylthio, etc. By way of illustration, the propylthio group signifies —SCH₂CH₂CH_{3.}

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydro-naphthyl, indanyl, biphenyl, phenanthryl, anthryl or acenaphthyl.

Unless otherwise defined, heteroaryl includes aromatic and partially aromatic groups which contain one or more heteroatoms. Examples of this type are thiophene, purine, imidazopyridine, pyridine, oxazole, thiazole, oxazine, pyrazole, tetrazole, imidazole, pyridine, pyrimidine, pyrazine and triazine. Examples of partially aromatic groups are tetrahydro-imidazo[4,5-c]pyridine, phthalidyl and saccharinyl, as defined below.

In situations in which a term occurs two or more times, the definition of the term in each occurrence is independent of the definition in each additional occurrence.

With respect to the compounds of formulas IV through XI the following definitions apply:

As used herein, “oligopeptide” is preferably a peptide comprising from about 5 amino acids to about 100 amino acids. More preferably, “oligopeptide” is a peptide comprising from about 5 amino acids to about 15 amino acids.

The terms “selective” and “selectively” as used in connection with recognition by PSA and the proteolytic PSA cleavage mean a greater rate of cleavage of an oligopeptide component of the instant invention by free PSA relative to cleavage of an oligopeptide which comprises a random sequence of amino acids. Therefore, the oligopeptide component of the instant invention is a preferred substrate of free PSA. The terms “selective” and “selectively” also indicate that the oligopeptide is proteolytically cleaved by free PSA between two specific amino acids in the oligopeptide.

As used herein, the term “hydroxylated” represents substitution on a substitutable carbon of the ring system being so described by a hydroxyl moiety. As used herein, the term “poly-hydroxylated” represents substitution on two or more substitutable carbon of the ring system being so described by 2, 3 or 4 hydroxyl moieties.

As used herein, the term “chlorosubstituted C₁-C₃-alkyl-CO-” represents a acyl moiety having the designated number of carbon atoms attached to a carbonyl moiety wherein one of the carbon atoms is substituted with a chlorine. Example of such chlorosubstituted elements include but are not limited to chloroacetyl, 2-chloropropionyl, 3-chloropropionyl and 2-chlorobutyroyl.

As used herein, the term “PEG” represents certain polyethylene glycol containing substituents having the designated number of ethyleneoxy subunits. Thus the term PEG(2) represents

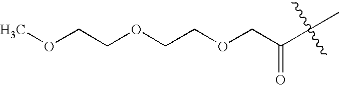

and the term PEG(6) represents

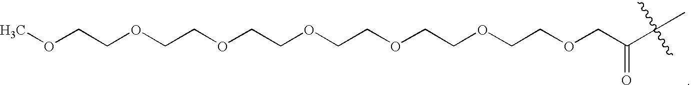

As used herein, the term “(d)(2,3-dihydroxypropionyl)” represents the following structure:

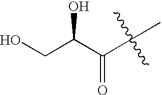

As used herein, the term “(2R,3S) 2,3,4-trihydroxybutanoyl” represents the following structure:

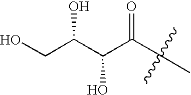

As used herein, the term “quinyl” represents the following structure:

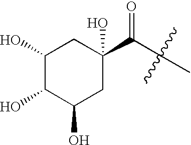

or the diastereomer thereof.

As used herein, the term “cotininyl” represents the following structure:

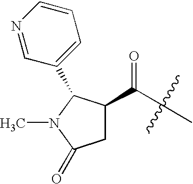

or the diastereomer thereof.

As used herein, the term “gallyl” represents the following structure:

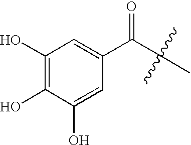

As used herein, the term “4-ethoxysquarate” represents the following structure:

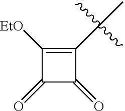

The structure

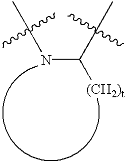

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

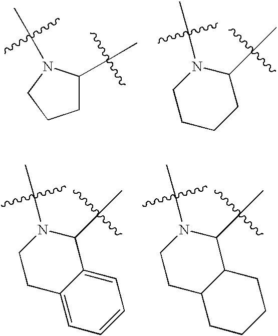

The instant invention comprises administering at least one conjugate, as discussed above, and at least one tachykinin receptor antagonist. The tachykinin receptor antagonists of use in the present invention may be any tachykinin antagonist known from the art. Preferably, the tachykinin receptor antagonist is a neurokinin-1 (NK-1) or neurokinin-2 (NK-2) receptor antagonist, especially a neurokinin-1 (NK-1) receptor antagonist.

The tachykinin antagonist may be peptidal or non-peptidal in nature, however, the use of a non-peptidal tachykinin receptor antagonist is preferred. In addition, for convenience the use of an orally active tachykinin receptor antagonist is preferred. An especially preferred class of NK-1 receptor antagonist of use in the present invention are those compounds which are orally active and long acting.

Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833 and 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514273, 0 514 274, 0 514275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are herein incorporated by reference.

Particularly preferred NK-1 receptor antagonists are those described in PCT International Patent Publication No. WO 95/16679 and European Patent Publication No. 0 577 394 as compounds of formula (IX):

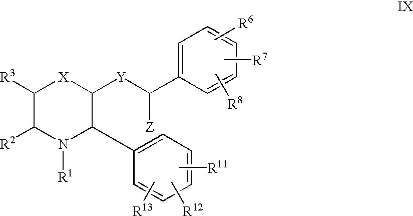

or a pharmaceutically acceptable salt thereof, wherein:

R¹ is selected from the group consisting of:

(1) hydrogen;

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo, wherein halo is fluoro, chloro, bromo or iodo,

(h) —NR⁹R¹⁰, wherein R⁹ and R¹⁰ are independently selected from:

(i) hydrogen,

(ii) C₁₋₆ alkyl,

(iii) hydroxy-C₁₋₆ alkyl, and

(iv) phenyl,

(i) —NR⁹COR¹⁰,

(j) —NR⁹CO₂R¹⁰,

(k) —CONR⁹R¹⁰,

(l) —COR⁹,

(m) —CO₂R⁹,

(n) heterocycle, wherein the heterocycle is selected from the group consisting of:

(A) benzimidazolyl,

(B) benzofuranyl,

(C) benzothiophenyl,

(D) benzoxazolyl,

(E) furanyl,

(F) imidazolyl,

(G) indolyl,

(H) isooxazolyl,

(I) isothiazolyl,

(J) oxadiazolyl,

(K) oxazolyl,

(L) pyrazinyl,

(M) pyrazolyl,

(N) pyridyl,

(O) pyrimidyl,

(P) pyrrolyl,

(Q) quinolyl,

(R) tetrazolyl,

(S) thiadiazolyl,

(T) thiazolyl,

(U) thienyl,

(V) triazolyl,

(W) azetidinyl,

(X) 1,4-dioxanyl,

(Y) hexahydroazepinyl,

(Z) piperazinyl,

(AA) piperidinyl,

(AB) pyrrolidinyl,

(AC) tetrahydrofuranyl, and

(AD) tetrahydrothienyl,

and wherein the heterocycle is unsubstituted or substituted with one or more substituent(s) selected from:

(i) C₁₋₆ alkyl, unsubstituted or substituted with halo, —CF₃, —OCH₃, or phenyl,

(ii) C₁₋₆ alkoxy,

(iii) oxo,

(iv) hydroxy,

(v) thioxo,

(vi) —SR⁹,

(vii) halo,

(viii) cyano,

(ix) phenyl,

(x) trifluoromethyl,

(xi) —(CH₂)ₘ—NR⁹R¹⁰, wherein m is 0, 1 or 2,

(xii) —NR⁹COR¹⁰,

(xiii) —CONR⁹R¹⁰,

(xiv) —CO₂R⁹, and

(xv) —(CH₂)ₘ—OR⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹,

(j) —CO₂R⁹,

(k) heterocycle;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹,

(o) —CO₂R⁹;

R² and R³ are independently selected from the group consisting of:

(1) hydrogen,

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

and the groups R¹ and R² may be joined together to form a heterocyclic ring selected from the group consisting of:

(a) pyrrolidinyl,

(b) piperidinyl,

(c) pyrrolyl,

(d) pyridinyl,

(e) imidazolyl,

(f) oxazolyl, and

(g) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹¹,

(v) halo, and

(vi) trifluoromethyl;

and the groups R² and R³ may be joined together to form a carbocyclic ring selected from the group consisting of:

(a) cyclopentyl,

(b) cyclohexyl,

(c) phenyl,

and wherein the carbocyclic ring is unsubstituted or substituted with one or more substituents selected from:

(i) C₁₋₆alkyl,

(ii) C₁₋₆alkoxy,

(iii) —NR⁹R¹⁰,

(iv) halo, and

(v) trifluoromethyl;

and the groups R² and R³ may be joined together to form a heterocyclic ring selected from the group consisting of:

(a) pyrrolidinyl,

(b) piperidinyl,

(c) pyrrolyl,

(d) pyridinyl,

(e) imidazolyl,

(f) furanyl,

(g) oxazolyl,

(h) thienyl, and

(i) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹⁰,

(v) halo, and

(vi) trifluoromethyl;

R⁶, R⁷ and R⁸ are independently selected from the group consisting of:

(1) hydrogen;

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹,

(o) —CO₂R⁹;

(6) halo,

(7) —CN,

(8) —CF₃,

(9) —NO₂,

(10) —SR¹⁴, wherein R¹⁴ is hydrogen or C₁₋₅alkyl,

(11) —SOR¹⁴,

(12) —SO₂R¹⁴,

(13) NR⁹COR¹⁰,

(14) CONR⁹COR¹⁰,

(15) NR⁹R¹⁰,

(16) NR⁹CO₂R¹⁰,

(17) hydroxy,

(18) C₁₋₆alkoxy,

(19) COR⁹,

(20) CO₂R⁹,

(21) 2-pyridyl,

(22) 3-pyridyl,

(23) 4-pyridyl,

(24) 5-tetrazolyl,

(25) 2-oxazolyl, and

(26) 2-thiazolyl;

R¹, R¹² and R 13 are independently selected from the definitions of R⁶, R⁷ and R⁸;

X is selected from the group consisting of:

(2) —S—,

(3) —SO—, and

(4) —SO₂—;

Y is selected from the group consisting of:

(1) a single bond,

(2) —O—,

(3) —S—,

(4) —CO—,

(5) —CH₂—,

(6) —CHR¹⁵—, and

(7) —CR¹⁵R¹⁶—, wherein R¹⁵ and R¹⁶ are independently selected from the group consisting of:

(a) C₁₋₆ alkyl, unsubstituted or substituted with one or more of the substituents selected from:

(i) hydroxy,

(ii) oxo,

(iii) C₁₋₆ alkoxy,

(iv) phenyl-C₁₋₃ alkoxy,

(v) phenyl,

(vi) —CN,

(vii) halo,

(viii) —NR⁹R¹⁰,

(ix) —NR⁹COR¹⁰,

(x) —NR⁹CO₂R¹⁰,

(xi) —CONR⁹R¹⁰,

(xii) —COR⁹, and

(xiii) —CO₂R⁹;

(b) phenyl, unsubstituted or substituted with one or more of the substituent(s) selected from:

(i) hydroxy,

(ii) C₁₋₆ alkoxy,

(iii) C₁₋₆ alkyl,

(iv) C₂₋₅ alkenyl,

(v) halo,

(vi) —CN,

(vii) —NO₂,

(viii) —CF₃,

(ix) —(CH₂)ₘ—NR⁹R¹⁰,

(x) —NR⁹COR¹⁰,

(xi) —NR⁹CO₂R¹⁰,

(xii) —CONR⁹R¹⁰,

(xiii) —CO₂NR⁹R¹⁰,

(xiv) —COR⁹, and

(xv) —CO₂R⁹; and

Z is C₁₋₆ alkyl.

Particularly preferred compounds of formula (IX) include:

4-(3-(1,2,4-triazolo)methyl)-2(S)-(3,5-bis(trifluoro-methyl)benzyloxy)-3(S)-phenyl-morpholine;

4-(3-(1,2,4-triazolo)methyl)-2(S)-(3,5-bis(trifluoro-methyl)benzyloxy)-3 (R)-phenyl-morpholine;

4-(3-(5-oxo-1H,4H-1,2,4-triazolo)methyl)-2(S)-(3,5-bis(trifluoromethyl)benzyloxy)-3(S)-phenyl-morpholine; and

2(R)-(1-(R)-(3,5-bis(trifluoromethyl)phenyl)ethoxy)-3-(S)(4-fluorophenyl)-4-(3-(5-oxo-1H,4H-1,2,4-triazolo)methyl)morpholine;

or a pharmaceutically acceptable salt thereof.

An especially preferred compound of formula (IX) is:

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methylmorpholine;

or a pharmaceutically acceptable salt thereof.

Further preferred NK-1 receptor antagonists are those described in PCT International Patent Publication No. WO 95/18124 as compounds of formula (X):

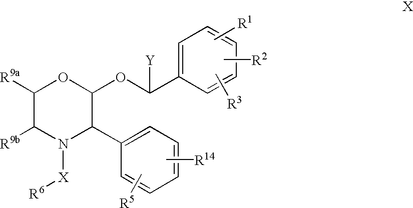

or a pharmaceutically acceptable salt or prodrug thereof, wherein

R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;

R² is hydrogen, halogen, C₁₋₆alkyl, C₁₋₆alkoxy substituted by C₁₋₄alkoxy or CF₃;

R³ is hydrogen, halogen or CF₃;

R⁴ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;

R⁵ is hydrogen, halogen, C₁₋₆alkyl, C₁₋₆alkoxy substituted by C₁₋₄alkoxy or CF₃;

R⁶ is a 5-membered or 6-membered heterocyclic ring containing 2 or 3 nitrogen atoms optionally substituted by ═O, ═S or a C₁₋₄alkyl group, and optionally substituted by a group of the formula ZNR⁷R⁸ where

Z is C₁₋₆alkylene or C₃₋₆cycloalkylene;

R⁷ is hydrogen, C₁₋₄alkyl, C₃₋₇cycloalkyl or C₃₋₇cycloalkylC₁₋₄ alkyl, or C₂₋₄alkyl substituted by C₁₋₄alkoxy or hydroxyl;

R⁸ is hydrogen, C₁₋₄alkyl, C₃₋₇cycloalkyl or C₃₋₇cycloalkylC₁₋₄ alkyl, or C₂₋₄alkyl substituted by one or two substituents selected from C₁₋₄alkoxy, hydroxyl or a 4, 5 or 6 membered heteroaliphatic ring containing one or two heteroatoms selected from N, O and S;

or R⁷, R⁸ and the nitrogen atom to which they are attached form a heteroaliphatic ring of 4 to 7 ring atoms, optionally substituted by a hydroxy group, and optionally containing a double bond, which ring may optionally contain an oxygen or sulphur ring atom, a group S(O) or S(O)₂ or a second nitrogen atom which will be part of a NH or NR^{c} moiety where R^{c} is C₁₋₄alkyl optionally substituted by hydroxy or C₁₋₄alkoxy;

or R⁷, R⁸ and the nitrogen atom to which they are attached form a non-aromatic azabicyclic ring system of 6 to 12 ring atoms;

or Z, R⁷ and the nitrogen atom to which they are attached form a heteroaliphatic ring of 4 to 7 ring atoms which may optionally contain an oxygen ring atom;

R^{9a} and R^{9b} are each independently hydrogen or C₁₋₄alkyl, or R^{9a} and R^{9b} are joined so, together with the carbon atoms to which they are attached, there is formed a C₅₋₇ ring;

X is an alkylene chain of 1 to 4 carbon atoms optionally substituted by oxo; and

Y is a C₁₋₄alkyl group optionally substituted by a hydroxyl group; with the proviso that if Y is C₁₋₄alkyl, R⁶ is substituted at least by a group of formula ZNR⁷R⁸ as defined above.

Particularly preferred compounds of formula (X) include:

2-(R)-2-(R)-(1-(R)-(3,5-bis(trifluoromethyl)phenyl)ethoxy)-4-(5-(dimethylamino) methyl-1,2,3-triazol-4-yl)methyl-3-(S)-phenylmorpholine;

(1-(R)-(3,5-bis(trifluoromethyl)phenyl)ethoxy)-4-((dimethylamino-methyl)-1,2,3-triazol-4-yl)methyl)-3-(S)-(4-fluorophenyl)morpholine;

or a pharmaceutically acceptable salt thereof.

Further preferred NK-1 receptor antagonists are those described in U.S. Pat. No. 5,691,336 and PCT International Patent Publication No.

WO 95/23798 as compounds of formula (XI):

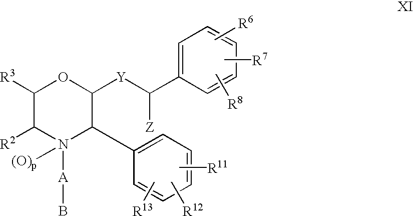

or a pharmaceutically acceptable salt thereof, wherein:

R² and R³ are independently selected from the group consisting of:

(1) hydrogen,

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰, wherein R⁹ and R¹⁰ are independently selected from:

(i) hydrogen,

(ii) C₁₋₆ alkyl,

(iii) hydroxy-C₁₋₆ alkyl, and

(iv) phenyl,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

and, alternatively, the groups R² and R³ are joined together to form a carbocyclic ring selected from the group consisting of:

(a) cyclopentyl,

(b) cyclohexyl,

(c) phenyl,

and wherein the carbocyclic ring is unsubstituted or substituted with one or more substituents selected from:

(i) C₁₋₆alkyl,

(ii) C₁₋₆alkoxy,

(iii) —NR⁹R¹⁰,

(iv) halo, and

(v) trifluoromethyl;

and, alternatively, the groups R² and R³ are joined together to form a heterocyclic ring selected from the group consisting of:

(a) pyrrolidinyl,

(b) piperidinyl,

(c) pyrrolyl,

(d) pyridinyl,

(e) imidazolyl,

(f) furanyl,

(g) oxazolyl,

(h) thienyl, and

(i) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹⁰,

(v) halo, and

(vi) trifluoromethyl;

R⁶, R⁷ and R⁸ are independently selected from the group consisting of:

(1) hydrogen;

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

(6) halo,

(7) —CN,

(8) —CF₃,

(9) —NO₂,

(10) —SR¹⁴, wherein R¹⁴ is hydrogen or C₁₋₅alkyl,

(11) —SOR¹⁴,

(12) —SO₂R¹⁴,

(13) NR⁹COR¹⁰,

(14) CONR⁹COR¹⁰,

(15) NR⁹R¹⁰,

(16) NR⁹CO₂R¹⁰,

(17) hydroxy,

(18) C₁₋₆alkoxy,

(19) COR⁹,

(20) CO₂R⁹,

(21) 2-pyridyl,

(22) 3-pyridyl,

(23) 4-pyridyl,

(24) 5-tetrazolyl,

(25) 2-oxazolyl, and

(26) 2-thiazolyl;

R¹, R¹² and R¹³ are independently selected from the definitions of R⁶, R⁷ and R⁸, or —OX;

A is selected from the group consisting of:

(1) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(2) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹; and

(3) C₂₋₆ alkynyl;

B is a heterocycle, wherein the heterocycle is selected from the group consisting of:

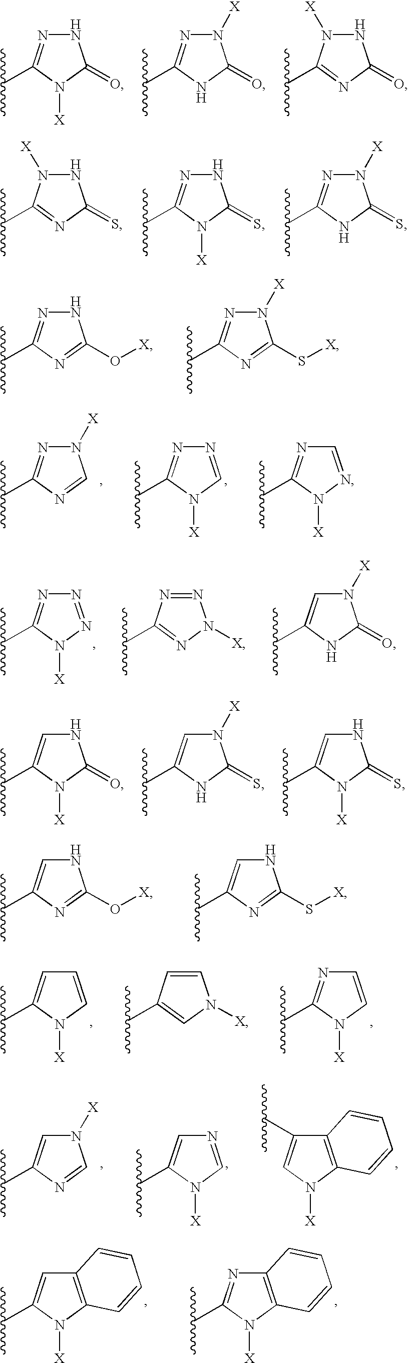

and wherein the heterocycle is substituted in addition to —X with one or more substituent(s) selected from:

(i) hydrogen;

(ii) C₁₋₆ alkyl, unsubstituted or substituted with halo, —CF₃, —OCH₃, or phenyl,

(iii) C₁₋₆ alkoxy,

(iv) oxo,

(v) hydroxy,

(vi) thioxo,

(vii) —SR⁹,

(viii) halo,

(ix) cyano,

(x) phenyl,

(xi) trifluoromethyl,

(xii) —(CH₂)ₘ—NR⁹R¹⁰, wherein m is 0, 1 or 2,

(xiii) —NR⁹COR¹⁰,

(xiv) —CONR⁹R¹⁰,

(xv) —CO₂R⁹, and

(xvi) —(CH₂)ₘ—OR⁹;

p is 0 or 1;

X is selected from:

(a) —PO(OH)O⁻.M⁺, wherein M⁺ is a pharmaceutically acceptable monovalent counterion,

(b) —PO(O⁻)₂.2M⁺,

(c) —PO(O⁻)₂. D²⁺, wherein D²⁺ is a pharmaceutically acceptable divalent counterion,

(d) —CH(R⁴)—PO(OH)O⁻.M⁺, wherein R⁴ is hydrogen or C₁₋₃ alkyl,

(e) —CH(R⁴)—PO(O⁻)₂.2M⁺,

(f) —CH(R⁴)—PO(O⁻)₂.D²⁺,

(g) —SO₃⁻.M+,

(h) —CH(R⁴)—SO₃⁻.M⁺,

(i) —CO—CH₂CH₂—CO₂—.M⁺,

(j) —CH(CH₃)—O—CO—R⁵, wherein R⁵ is selected from the group consisting of:

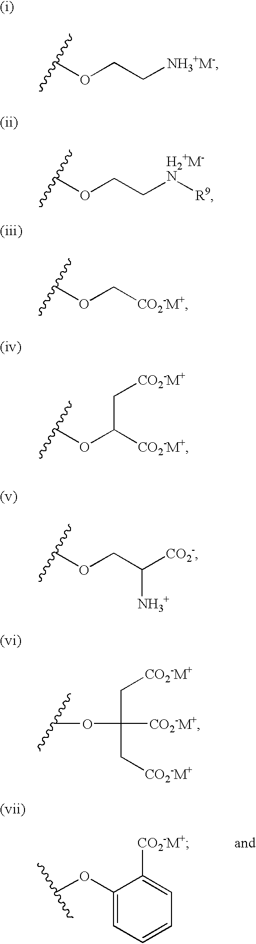

(k) hydrogen, with the proviso that if p is 0 and none of R¹¹, R¹² or R¹³ are —OX, then X is other than hydrogen;

Y is selected from the group consisting of:

(1) a single bond,

(2) —O—,

(3) —S—,

(4) —CO—,

(5) —CH₂—,

(6) —CHR¹⁵—, and

(7) —CR¹⁵R¹⁶—, wherein R¹⁵ and R¹⁶ are independently selected from the group consisting of:

(a) C₁₋₆ alkyl, unsubstituted or substituted with one or more of the substituents selected from:

(i) hydroxy,

(ii) oxo,

(iii) C₁₋₆ alkoxy,

(iv) phenyl-C₁₋₃ alkoxy,

(v) phenyl,

(vi) —CN,

(vii) halo,

(viii) —NR⁹R¹⁰,

(ix) —NR⁹COR¹⁰,

(x) —NR⁹CO₂R¹¹,

(xi) —CONR⁹R¹⁰,

(xii) —COR⁹, and

(xiii) —CO₂R⁹;

(b) phenyl, unsubstituted or substituted with one or more of the substituent(s) selected from:

(i) hydroxy,

(ii) C₁₋₆ alkoxy,

(iii) C₁₋₆ alkyl,

(iv) C₂₋₅ alkenyl,

(v) halo,

(vi) —CN,

(vii) —NO₂,

(viii) —CF₃,

(ix) —(CH₂)ₘ—NR⁹R¹⁰,

(x) —NR⁹COR¹⁰,

(xi) —NR⁹CO₂R¹⁰,

(xii) —CONR⁹R¹⁰,

(xiii) —CO₂NR⁹R¹⁰,

(xiv) —COR⁹, and

(xv) —CO₂R⁹;

Z is selected from:

(1) hydrogen,

(2) C₁₋₆ alkyl, and

(3) hydroxy, with the proviso that if Y is —O—, Z is other than hydroxy, or if Y is —CHR¹⁵—, then Z and R¹⁵ are optionally joined together to form a double bond.

A particularly preferred compound of formula (XI) is 2-(R)-(1-(R)-(3,5-bis(trifluoromethyl)phenyl)ethoxy)-3-(S)-(4-fluoro)-phenyl-4-(3-(1-monophosphoryl-5-oxo-1H-1,2,4-triazolo)methyl)morpholine, or a pharmaceutically acceptable salt thereof. In particular, the bis(N-methyl-D-glucamine) salt is preferred.

Further preferred NK-1 receptor antagonists are those described in European Patent Specification No. WO 96/05181, i.e. compounds of formula (XII):

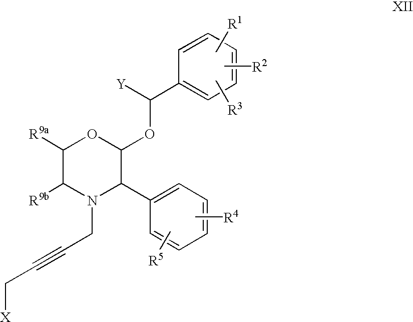

wherein:

X is a group of the formula NR⁶R⁷ or a C— or N-linked imidazolyl ring;

Y is hydrogen or C₁₋₄alkyl optionally substituted by a hydroxy group;

R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} each independently represent hydrogen or C₁₋₄alkyl;

R² is hydrogen, halogen, C₁₋₆alkyl, C₁₋₆alkoxy substituted by C₁₋₄ alkoxy or CF₃;

R³ is hydrogen, halogen or CF³;

R⁴ is hydrogen, halogen, C₁₋₆alkyl, C₁₋₆alkoxy, hydroxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} are as previously defined;

R⁵ is hydrogen, halogen, C₁₋₆alkyl, C₁₋₆alkoxy substituted by C₁₋₄ alkoxy or CF₃;

R⁶ is hydrogen, C₁₋₆alkyl, C₃₋₇cycloalkyl, C₃₋₇cycloalkylC₁₋₄alkyl, phenyl, or C₂₋₄alkyl substituted by C₁₋₄alkoxy or hydroxy;

R⁷ is hydrogen, C₁₋₆alkyl, C₃₋₇cycloalkyl, C₃₋₇cycloalkylC₁₋₄alkyl, phenyl, or C₂₋₄alkyl substituted by one or two substituents selected from C₁₋₄alkoxy, hydroxy or a 4, 5 or 6 membered heteroaliphatic ring containing one or two heteroatoms selected from N, O and S;

or R⁶ and R⁷, together with the nitrogen atom to which they are attached, form a saturated or partially saturated heterocyclic ring of 4 to 7 ring atoms, which ring may optionally contain in the ring one oxygen or sulphur atom or a group selected from NR⁸, S(O) or S(O)₂ and which ring may be optionally substituted by one or two groups selected from hydroxyC₁₋₄alkyl, C₁₋₄alkoxyC₁₋₄alkyl, oxo, COR^{a} or CO₂R^{a} where R^{a} is as previously defined;

or R⁶ and R⁷ together with the nitrogen atom to which they are attached, form a non-aromatic azabicyclic ring system of 6 to 12 ring atoms;

R⁸ is hydrogen, C₁₋₄alkyl, hydroxyC₁₋₄alkyl or C₁₋₄alkoxyC₁₋₄ alkyl; and

R^{9a} and R^{9b} are each independently hydrogen or C₁₋₄alkyl, or R^{9a} and R^{9b} are joined so, together with the carbon atoms to which they are attached, there is formed a C₅₋₇ ring;

and pharmaceutically acceptable salts thereof.

Specific compounds of formula (XII) of use in the present invention include:

2-(R)-(1-(R)-(3,5-bis(trifluoromethyl)phenyl)ethoxy)-3-(S)-(4-fluorophenyl)-4-(4-morpholinobut-2-yn-yl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

4-(4-azetidinylbut-2-yn-yl)-2-(R)-(1-(R)-(3,5-bis(trifluoromethyl)phenyl) ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-imidazolylbut-2-yn-yl)morpholine;

2-(R)-(1-(R)-(3,5-bis(trifluoromethyl)phenyl)ethoxy)-3-(S)-(4-fluorophenyl)-4-(4-(N-methylpiperazinyl)but-2-yn-yl)morpholine;

4-(4-bis(2-methoxyethyl)aminobut-2-yn-yl)-2-(R)-(1-(R)-(3,5-bis(trifluoromethyl) phenyl)ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-pyrrolidinobut-2-yn-yl)morpholine;

3-(S)-(4-fluorophenyl)-2-(R)-(1-(R)-(3-fluoro-5-(trifluoromethyl)phenyl)ethoxy)-4-(4-morpholinobut-2-yn-yl)morpholine;

3-(S)-(4-fluorophenyl)-4-(4-morpholinobut-2-yn-yl)-2-(R)-(1-(R)-(3-(trifluoromethyl) phenyl)ethoxy)morpholine;

4-(4-azetidinylbut-2-yn-yl)-3-(S)-(4-fluorophenyl)-2-(R)-(1-(R)-(3-(trifluoromethyl) phenyl)ethoxy)morpholine;

2-(R)-(1-(R)-(3,5-bis(trifluoromethyl)phenyl)ethoxy)-4-(4-(N-(2-methoxyethyl)-N-methyl)aminobut-2-yn-yl)-3-(S)-phenylmorpholine;

2-(R)-(1-(R)-(3,5-bis(trifluoromethyl)phenyl)ethoxy)-4-(4-(N-cyclopropyl-N-(2-methoxyethyl)amino)but-2-yn-yl)-3-(S)-phenylmorpholine;

2-(R)-(1-(R)-(3,5-bis(trifluoromethyl)phenyl)ethoxy)-4-(4-(N-isopropyl-N-(2-methoxyethyl)amino)but-2-yn-yl)-3-(S)-phenylmorpholine;

4-(4-(N,N-dimethylamino)but-2-yn-yl)-3-(S)-(4-fluorophenyl)-2-(R)-(1-(S)-(3-fluoro-5-(trifluoromethyl)phenyl-2-hydroxyethoxy)morpholine;

4-(4-azetidinylbut-2-yn-yl)-3-(S)-(4-fluorophenyl)-2-(R)-(1-(S)-(3-fluoro-5-(trifluoromethyl)phenyl)-2-hydroxyethoxy)morpholine;

2-(R)-(1-(S)-(3,5-bis(trifluoromethyl)phenyl)-2-hydroxyethoxy)-4-(4-(N,N-dimethylamino)but-2-yn-yl)-3-(S)-(4-fluorophenyl)morpholine;

4-(4-azetidinylbut-2-yn-yl)-2-(R)-(1-(S)-(3,5-bis(trifluoromethyl)phenyl-2-hydroxyethoxy)-3-(S)-(4-fluorophenyl)morpholine;

4-(4-N-bis(2-methoxy)ethyl-N-methylamino)but-2-yn-yl)-2-(R)-(1-(R)-(3,5-bis(trifluoromethyl)phenyl)ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-(2-(S)-(methoxymethyl)pyrrolidino)but-2-yn-yl)morpholine;

4-(4-(7-azabicyclo[2.2.1]heptano)but-2-yn-yl)-2-(R)-(1-(R)-(3,5-bis(trifluoromethyl) phenyl)ethoxy)-3-(S)-(4-fluorophenyl)morpholine;

2-(R)-(1-(R)-(3,5-bis(trifluoromethyl)phenyl)ethoxy)-4-(4-diisopropylaminobut-2-yn-yl)-3-(S)-(4-fluorophenyl)morpholine;

2-(R)-(1-(R)-(3-fluoro-5-(trifluoromethyl)phenyl)ethoxy)-4-(4-(2-(S)-(methoxymethyl)pyrrolidino)but-2-yn-yl)-3-(S)-phenylmorpholine;

2-(R)-(1-(R)-(3,5-bis(trifluoromethyl)phenyl)ethoxy)-3-(S)-(4-fluorophenyl)-4-(4-(2-(S)-(hydroxymethyl)pyrrolidino)but-2-yn-yl)morpholine;

and pharmaceutically acceptable salts thereof.

Further preferred NK-1 receptor antagonists are those described in European Patent Publication No. 0 436 334 as compounds of formula (XIII):

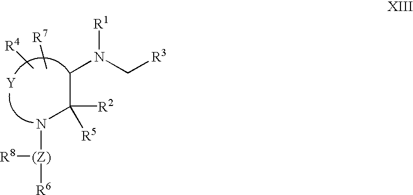

or a pharmaceutically acceptable salt thereof, wherein

Y is (CH₂)ₙ wherein n is an integer from 1 to 4, and wherein any one of the carbon-carbon single bonds in said (CH₂)ₙ may optionally be replaced by a carbon-carbon double bond, and wherein any one of the carbon atoms of said (CH₂)ₙ may optionally be substituted with R⁴, and wherein any one of the carbon atoms of said (CH₂)ₙ may optionally be substituted with R⁷;

Z is (CH₂)ₘ wherein m is an integer from 0 to 6, and wherein any one of the carbon-carbon single bonds of (CH₂)ₘ may optionally be replaced by a carbon-carbon double bond or a carbon-carbon triple bond, and any one of the carbon atoms of said (CH₂)ₘ may optionally be substituted with R⁸;

R¹ is hydrogen or C₁₋₈alkyl optionally substituted with hydroxy, C₁₋₄ alkoxy or fluoro;

R² is a radical selected from hydrogen, C₁₋₆ straight or branched alkyl, C₃₋₇cycloalkyl wherein one of the CH₂ groups in said cycloalkyl may optionally be replaced by NH, oxygen or sulphur; aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; phenyl-C₂₋₆ alkyl, benzhydryl and benzyl, wherein each of said aryl and heteroaryl groups and the phenyl moieties of said benzyl, phenyl —C₂₋₆alkyl and benzhydryl may optionally be substituted with one or more substituents independently selected from halo, nitro, C₁₋₆alkyl, C₁₋₆ alkoxy, trifluoromethyl, amino, C₁₋₆alkylamino, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆ alkyl-CO, C₁₋₆alkyl-C₁₋₆alkyl-, di-C₁₋₆alkylamino, —CONH—C₁₋₆alkyl, C₁₋₆ alkyl-CO—NH—C₁₋₆alkyl, —NHCOH and —NHCO—C₁₋₆alkyl; and wherein one of the phenyl moieties of said benzhydryl may optionally be replaced by naphthyl, thienyl, furyl or pyridyl;

R⁵ is hydrogen, phenyl or C₁₋₆alkyl;

or R² and R⁵ together with the carbon to which they are attached, form a saturated ring having from 3 to 7 carbon atoms wherein one of the CH₂ groups in said ring may optionally be replaced by oxygen, NH or sulfur;

R³ is aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; and cycloalkyl having 3 to 7 carbon atoms wherein one of the (CH₂) groups in said cycloalkyl may optionally be replaced by NH, oxygen or sulphur;

wherein each of said aryl and heteroaryl groups may optionally be substituted with one or more substituents, and said C₃₋₇cycloalkyl may optionally be substituted with one or two substituents, each of said substituents being independently selected from halo, nitro, C₁₋₆alkyl, C₁₋₆alkoxy, trifluoromethyl, amino, C₁₋₆alkylamino, —CO—NH—C₁₋₆alkyl, C₁₋₆alkyl-CO—NH—C₁₋₆alkyl, —NHCOH and —NH CO—C₁₋₆alkyl;

R⁴ and R⁷ are each independently selected from hydroxy, halogen, halo, amino, oxo, cyano, methylene, hydroxymethyl, halomethyl, C₁₋₆alkylamino, di-C₁₋₆alkylamino, C₁₋₆alkoxy, C₁₋₆alkyl-O—CO, C₁₋₆alkyl-O—CO—C₁₋₆alkyl, C₁₋₆ alkyl-CO—O, C₁₋₆alkyl-CO—C₁₋₆alkyl-O—, C₁₋₆alkyl-CO—, C₁₋₆alkyl-CO—C₁₋₆alkyl, and the radicals set forth in the definition of R²;

R⁶ is —NHCOR⁹, —NHCH₂R⁹, SO₂R⁸ or one of the radicals set forth in any of the definitions of R², R⁴ and R⁷;

R⁸ is oximino (═NOH) or one of the radicals set forth in any of the definitions of R², R⁴ and R⁷;

R⁹ is C₁₋₆alkyl, hydrogen, phenyl or phenylC₁₋₆alkyl; with the proviso that (a) when m is 0, R⁸ is absent, (b) when R⁴, R⁶, R⁷ or R⁸ is as defined in R², it cannot form together with the carbon to which it is attached, a ring with R⁵, and (c) when R⁴ and R⁷ are attached to the same carbon atom, then either each of R⁴ and R⁷ is independently selected from hydrogen, fluoro and C₁₋₆alkyl, or R⁴ and R⁷, together with the carbon to which they are attached, for a C₃₋₆ saturated carbocyclic ring that forms a spiro compound with the nitrogen-containing ring to which they are attached.

A particularly preferred compound of formula (XIII) is (2S,3S)-cis-3-(2-methoxybenzylamino)-2-phenylpiperidine; or a pharmaceutically acceptable salt thereof.

Another class of NK-1 receptor antagonists is as disclosed in PCT International Patent Publication No. WO 93/21155 as compounds of formula (XIV):

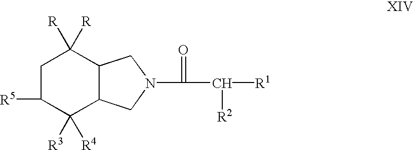

or a pharmaceutically acceptable salt thereof, wherein

radicals R are phenyl radicals optionally 2- or 3-substituted by a halogen atom or a methyl radical;

R¹ is optionally substituted phenyl, cyclohexadienyl, naphthyl, indenyl or optionally substituted heterocycle;

R² is H, halogen, OH, alkyl, aminoalkyl, alkylaminoalkyl, dialkylaminoalkyl, alkyloxy, alkylthio, acyloxy, carboxy, optionally substituted alkyloxycarbonyl, benzyloxycarbonyl, amino or acylamino;

R³ is optionally 2-substituted phenyl;

R⁴ is OH or fluorine when R⁵ is H;

or R⁴ and R⁵ are OH;

or R⁴ and R⁵ together form a bond.

A particularly preferred compound of formula (XIV) is (3aS,4S,7aS)-7,7-diphenyl-4-(2-methoxyphenyl)-2-[(2S)-(2-methoxy-phenyl)propionyl] perhydroisoindol-4-ol; or a pharmaceutically acceptable salt thereof.

Another class of NK-1 receptor antagonists of use in the present invention is that described in European Patent Publication No. 0 591 040 as compounds of formula (XV):

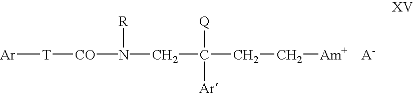

wherein

Ar represents an optionally substituted mono-, di- or tricyclic aromatic or heteroaromatic group;

T represents a bond, a hydroxymethylene group, a C₁₋₄ alkoxymethylene group or a C₁₋₅alkylene group;

Ar′ represents a phenyl group which is unsubstituted or substituted by one or more substituents selected from halogen, preferably chlorine or fluorine, trifluoromethyl, C₁₋₄alkoxy, C₁₋₄alkyl where the said substituents may be the same or different; a thienyl group; a benzothienyl group; a naphthyl group; or an indolyl group;

R represents hydrogen, C₁₋₄alkyl, —C₁₋₄alkoxyC₁₋₄alkyl, or —C₂₋₄ alkanoyloxyC₂₋₄alkyl;

Q represents hydrogen;

or Q and R together form a 1,2-ethylene, 1,3-propylene or 1,4-butylene group;

Am⁺ represents the radical

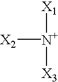

in which X₁, X₂ and X₃, together with the nitrogen atom to which they are attached, form an azabicyclic or azatricyclic ring system optionally substituted by a phenyl or benzyl group; and

A⁻ represents a pharmaceutically acceptable anion.

A particularly preferred compound of formula (XV) is (+) 1-[2-[3-(3,4-dichlorophenyl)-1-[(3-isopropoxyphenyl)acetyl]-3-piperidinyl]ethyl]-4-phenyl-1-azabicyclo[2,2,2]octane; or a pharmaceutically acceptable salt, especially the chloride, thereof.

Another class of NK-1 receptor antagonists of use in the present invention is that described in European Patent Publication No. 0 532 456 as compounds of formula (XVI):

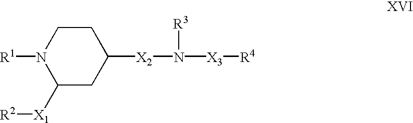

or a pharmaceutically acceptable salt thereof, wherein

R¹ represents an optionally substituted aralkyl, aryloxyalkyl, heteroaralkyl, aroyl, heteroaroyl, cycloalkylcarbonyl, aralka noyl, heteroarylalkanoyl, aralkoxycarbonyl or arylcarbamoyl group or the acyl group of an (-amino acid optionally N-substituted by a lower alkanoyl or carbamoyl-lower alkanoyl group;

R² represents cycloalkyl or an optionally substituted aryl or heteroaryl group;

R³ represents hydrogen, alkyl, carbamoyl or an alkanoyl or alkenoyl group optionally substituted by carboxy or esterified or amidated carboxy;

R⁴ represents an optionally substituted aryl group or an optionally partially saturated heteroaryl group;

X₁ represents methylene, ethylene, a bond, an optionally ketalised carbonyl group or an optionally etherified hydroxymethylene group;

X₂ represents alkylene, carbonyl or a bond; and

X₃ represents carbonyl, oxo-lower alkyl, oxo(aza)-lower alkyl, or an alkyl group optionally substituted by phenyl, hydroxymethyl, optionally esterified or amidated carboxy, or (in other than the (-position) hydroxy.

A particularly preferred compound of formula (XVI) is (2R*,4S*)-2-benzyl-1-(3,5-dimethylbenzoyl)-N-(4-quinolinylmethyl)-4-piperidineamine; or a pharmaceutically acceptable salt thereof.

Another class of NK-1 receptor antagonists of use in the present invention is that described in European Patent Publication No. 0 443 132 as compounds of formula (XVII):

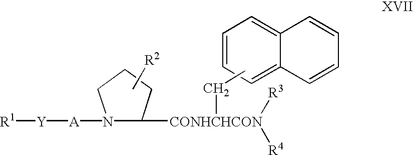

or a pharmaceutically acceptable salt thereof,

wherein R¹ is aryl, or a group of the formula:

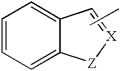

or a pharmaceutically acceptable salt thereof, wherein

X is CH or N;

Z is O or N—R⁵, in which R⁵ is hydrogen or lower alkyl;

R² is hydroxy or lower alkoxy;

R³ is hydrogen or optionally substituted lower alkyl;

R⁴ is optionally substituted ar(lower) alkyl;

A is carbonyl or sulfonyl; and

Y is a bond or lower alkenylene.

A particularly preferred compound of formula (XVII) is the compound of formula (XVIIa)

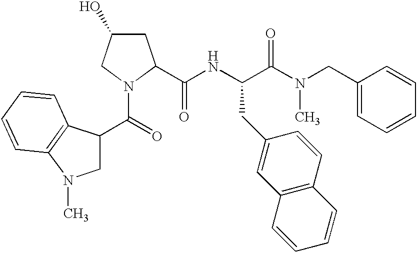

Another class of NK-1 receptor antagonists of use in the present invention is that described in PCT International Patent Publication No. WO 92/17449 as compounds of the formula (XVIII):

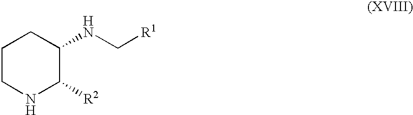

or a pharmaceutically acceptable salt thereof, wherein

R¹ is aryl selected from indanyl, phenyl and naphthyl; heteroaryl selected from thienyl, furyl, pyridyl and quinolyl; and cycloalkyl having 3 to 7 carbon atoms, wherein one of said carbon atoms may optionally be replaced by nitrogen, oxygen or sulfur; wherein each of said aryl and heteroaryl groups may optionally be substituted with one or more substituents, and said C₃₋₇cycloalkyl may optionally be substituted with one or two substituents, said substituents being independently selected from chloro, fluoro, bromo, iodo, nitro, C₁₋₁₀alkyl optionally substituted with from one to three fluoro groups, C₁₋₁₀alkoxy optionally substituted with from one to three fluoro groups, amino, C₁₋₁₀alkyl-S—, C₁₋₁₀alkyl-S(O)—, C₁₋₁₀alkyl-SO₂—, phenyl, phenoxy, C₁₋₁₀alkyl-SO₂NH—, C₁₋₁₀alkyl-SO₂NH-C₁₋₁₀alkyl-, C₁₋₁₀alkylamino-diC₁₋₁₀alkyl-, cyano, hydroxy, cycloalkoxy having 3 to 7 carbon atoms, C₁₋₆alkylamino, C₁₋₆ dialkylamino, HC(O)NH— and C₁₋₁₀alkyl-C(O)NH—; and

R² is thienyl, benzhydryl, naphthyl or phenyl optionally substituted with from one to three substituents independently selected from chloro, bromo, fluoro, iodo, cycloalkoxy having 3 to 7 carbon atoms, C₁₋₁₀alkyl optionally substituted with from one to three fluoro groups and C₁₋₁₀alkoxy optionally substituted with from one to three fluoro groups.

A particularly preferred compound of formula (XVIII) is (2S,3S)-3-(2-methoxy-5-trifluoromethoxybenzyl)-amino-2-phenylpiperidine; or a pharmaceutically acceptable salt thereof.

Another class of NK-1 receptor antagonists of use in the present invention is that described in PCT International Patent Publication No. WO 95/08549 as compounds of formula (XIX):

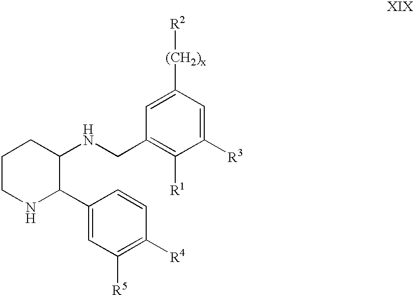

wherein R¹ is a C₁₋₄alkoxy group;

R² is

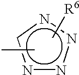

R³ is a hydrogen or halogen atom;

R⁴ and R⁵ may each independently represent a hydrogen or halogen atom, or a C₁₋₄ alkyl, C₁₋₄ alkoxy or trifluoromethyl group;

R⁶ is a hydrogen atom, a C₁₋₄ alkyl, (CH₂)ₘ cyclopropyl, —S(O)ₙC₁₋₄ alkyl, phenyl, NR⁷R⁸, CH₂C(O)CF₃ or trifluoromethyl group;

R⁷ and R⁸ may each independently represent a hydrogen atom, or a C₁₋₄ alkyl or acyl group;

x represents zero or 1;

n represents zero, 1 or 2;

m represents zero or 1;

and pharmaceutically acceptable salts and solvates thereof.

A particularly preferred compound of formula (XIX) is [2-methoxy-5-(5-trifluoromethyl-tetrazol-1-yl)-benzyl]-(2S-phenyl-piperidin-3S-yl)-amine; or a pharmaceutically acceptable salt thereof.

Another class of NK-1 receptor antagonists of use in the present invention is that described in PCT International Patent Publication No. WO 97/49710 as compounds of formula (XX):

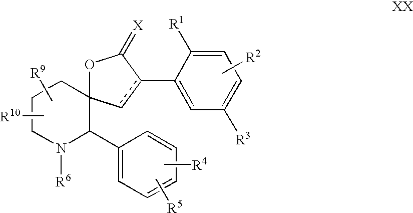

wherein R¹, R², R³, R⁴, R⁵, R⁶, R⁹, R¹⁰, and X are as defined therein.

Particularly preferred compounds of formula (XX) are

(3S,5R,6S)-3-[2-cyclopropoxy-5-(trifluoromethoxy)phenyl]-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane; or a pharmaceutically acceptable salt thereof.

Another class of tachykinin receptor antagonists of use in the present invention is that described in PCT International Patent Publication No. WO 95/06645 as compounds of formula (XXI):

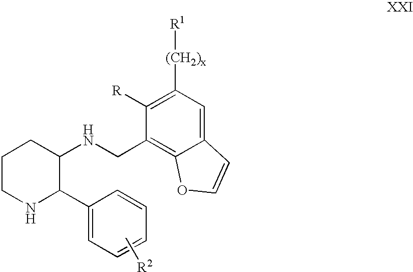

wherein

R represents a hydrogen atom or a C₁₋₄ alkoxy group;

R¹ is selected from phenyl, optionally substituted by a group —(CH₂)ₙCONR³R⁴ or S(O)ₘR³; or a 5- or 6-membered aromatic heterocycle containing 1, 2, 3 or 4 heteroatoms selected from oxygen, nitrogen, or sulphur, optionally substituted by a C₁₋₄ alkyl, trifluoromethyl or cyano group or a group —(CH₂)ₙCONR³R⁴;

R² represents a hydrogen or halogen atom;

R³ and R⁴ independently represent hydrogen or C₁₋₄ alkyl;

n represents zero, 1 or 2;

m represents zero, 1 or 2;

z represents zero or 1;

and pharmaceutically acceptable salts and solvates thereof.

A particularly preferred compound of formula (XXI) is [5-(5-methyl-tetrazol-1-yl)-benzofuran-7-ylmethyl]-(2S-phenyl-piperidin-3S-yl)-amine; or a pharmaceutically acceptable salt thereof.

Another class of tachykinin receptor antagonists of use in the present invention is that described in PCT International Patent Publication No. WO 95/14017, i.e. compounds of formula (XXII)

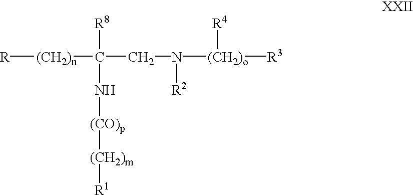

or a pharmaceutically acceptable salt thereof, wherein

m is zero, 1, 2 or 3;

n is zero or 1;

o is zero, 1 or 2;

p is zero or 1;

R is phenyl, 2- or 3-indolyl, 2- or 3-indolinyl, benzothienyl, benzofuranyl, or naphthyl;

which R groups may be substituted with one or two halo, C₁₋₃alkoxy, trifluoromethyl, C₁₋₄alkyl, phenyl-C₁₋₃alkoxy, or C₁₋₄alkanoyl groups;

R¹ is trityl, phenyl, diphenylmethyl, phenoxy, phenylthio, piperazinyl, piperidinyl, pyrrolidinyl, morpholinyl, indolinyl, indolyl, benzothienyl, hexamethyleneiminyl, benzofuranyl, tetrahydropyridinyl, quinolinyl, isoquinolinyl, reduced quinolinyl, reduced isoquinolinyl, phenyl-(C₁₋₄alkyl)-, phenyl-(C₁₋₄alkoxy)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-(C₁₋₃alkyl)-, C₁₋₄alkyl, or —NH—CH₂—R⁵;

any one of which R¹ groups may be substituted with halo, C₁₋₄alkyl, C₁₋₄alkoxy, trifluoromethyl, amino, C₁₋₄alkylamino, di(C₁₋₄alkyl)amino, or C₂₋₄ alkanoylamino;

or any one of which R¹ groups may be substituted with phenyl, piperazinyl, C₃₋₈cycloalkyl, benzyl, C₁₋₄alkyl, piperidinyl, pyridinyl, pyrimidinyl, C₂₋₆ alkanoylamino, pyrrolidinyl, C₂₋₆alkanoyl, or C₁₋₄alkoxycarbonyl;

any one of which groups may be substituted with halo, C₁₋₄alkyl, C₁₋₄alkoxy, trifluoromethyl, amino, C₁₋₄alkylamino, di(C₁₋₄alkyl)amino, or C₂₋₄ alkanoylamino;

or R¹ is amino, a leaving group, hydrogen, C₁₋₄alkylamino, or di(C₁₋₄alkyl)amino;

R⁵ is pyridyl, anilino-(C₁₋₃alkyl)-, or anilinocarbonyl;

R² is hydrogen, C₁₋₄alkyl, C₁₋₄alkylsulfonyl, carboxy-(C₁₋₃alkyl)-, C₁₋₃alkoxycarbonyl-(C₁₋₃alkyl)-, or —CO—R⁶;

R⁶ is hydrogen, C₁₋₄alkyl, C₁₋₃haloalkyl, phenyl, C₁₋₃alkoxy, C₁₋₃hydroxyalkyl, amino, C₁₋₄alkylamino, di(C₁₋₄alkyl)amino, or —(CH₂)_{q}—R⁷;

q is zero to 3;

R⁷ is carboxy, C₁₋₄alkoxycarbonyl, C₁₋₄alkylcarbonyloxy, amino, C₁₋₄alkylamino, di(C₁₋₄alkyl)amino, C₁₋₆alkoxycarbonylamino, or phenoxy, phenylthio, piperazinyl, piperidinyl, pyrrolidinyl, morpholinyl, indolinyl, indolyl, benzothienyl, benzofuranyl, quinolinyl, phenyl-(C₁₋₄alkyl)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-C₁₋₃alkyl;

any one of which aryl or heterocyclic R⁷ groups may be substituted with halo, trifluoromethyl, C₁₋₄alkoxy, C₁₋₄alkyl, amino, C₁₋₄alkylamino, di(C₁₋₄alkyl) amino, or C₂₋₄alkanoylamino;

or any one of which R⁷ groups may be substituted with phenyl, piperazinyl, C₃₋₈cycloalkyl, benzyl, piperidinyl, pyridinyl, pyrimidinyl, pyrrolidinyl, C₂₋₆alkanoyl, or C₁₋₄alkoxycarbonyl;

any of which groups may be substituted with halo, trifluoromethyl, amino, C₁₋₄alkoxy, C₁₋₄alkyl, C₁₋₄alkylamino, di(C₁₋₄alkyl)amino, or C₂₋₄alkanoylamino;

R⁸ is hydrogen or C₁₋₆alkyl;

R³ is phenyl, phenyl-(C₁₋₆alkyl)-, C₃₋₈cycloalkyl, C₅₋₈cycloalkenyl, C₁₋₈alkyl, naphthyl, C₂₋₈alkenyl, or hydrogen;

any one or which groups except hydrogen may be substituted with one or two halo, C₁₋₃alkoxy, C₁₋₃alkylthio, nitro, trifluoromethyl, or C₁₋₃alkyl groups; and

R⁴ is hydrogen or C₁₋₃alkyl;

with the proviso that if R¹ is hydrogen or halo, R³ is phenyl, phenyl-(C₁₋₆alkyl)-, C₃₋₈cycloalkyl, C₅₋₈cycloalkenyl, or naphthyl.

A particularly preferred compound of formula (XXII) is [N-(2-methoxybenzyl)acetylamino]-3-(1H-indol-3-yl)-2-[N-(2-(4-piperidin-1-yl)piperidin-1-yl)acetylamino]propane; or a pharmaceutically acceptable salt thereof.

The preferred compounds of formulae (IX), (X), (XI), and (XII) will have the 2- and 3-substituents on the morpholine ring in the cis arrangement, the preferred stereochemistry being as shown in the following general formula:

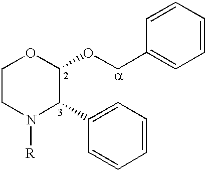

Where the benzyloxy moiety is α-substituted, the preferred stereochemistry of the α-carbon is either (R) when the substituent is an alkyl (e.g. methyl) group or (S) when the substituent is a hydroxyalkyl (e.g. hydroxymethyl) group.

The preparations of the foregoing compounds are fully described in the referenced patents and publications.

The compounds of formulae (IX) through (XXII) may be prepared by the methods described in EP-A-0 577 394 (or WO 95/16679), WO 95/18124, WO 95/23798, WO 96/05181, EP-A-0 436 334, WO 93/21155, EP-A-0 591 040, EP-A-0 532 456, EP-A-0 443 132, WO 92/17449, WO 95/08549, WO 97/49710, WO 95/06645 and WO 95/14017, respectively.

Preferred NK-1 receptor antagonists for use in the present invention as orally active, long acting, CNS-penetrant NK-1 receptor antagonists are selected from the classes of compounds described in European Patent Specification No. 0 577 394, and International Patent Specification Nos. 95/08549, 95/18124, 95/23798, 96/05181 and WO 97/49710.

Thus, further particularly preferred NK-1 receptor antagonists of use in the present invention include:

(±)-(2R, 3R, 2S3S)-N-{[2-cyclopropoxy-5-(trifluoromethoxy)-phenyl]methyl}-2-phenylpiperidin-3-amine;

2-(S)-(3,5-bis(trifluoromethyl)benzyloxy)-3(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;

2-(S)-(3,5-bis(trifluoromethyl)benzyloxy)-4-(3-(5-oxo-1H,4H-1,2,4-triazolo)methyl)-3-(S)-phenyl-morpholine;

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(5-(N,N-dimethylamino) methyl-1,2,3-triazol-4-yl)methyl-3-(S)-phenylmorpholine;

2-(R)-(1-(R)-(3,5-bis(trifluoromethyl)phenyl)ethoxy)-4-(5-(N,N-dimethylamino) methyl-1,2,3-triazol-4-yl)methyl-3-(S)-(4-fluorophenyl)morpholine;

(3S,5R,6S)-3-[2-cyclopropoxy-5-(trifluoromethoxy)phenyl]-6-phenyl-1-oxa-7-aza-spiro[4.5]decane; (3R,5R,6S)-3-[2-cyclopropoxy-5-(trifluoromethoxy)phenyl]-6-phenyl-1-oxa-7-aza-spiro[4.5]decane;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;

2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;

2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

or a pharmaceutically acceptable salt thereof.

Particularly preferred NK-1 receptor antagonists of the formulae (IX) through (XXII) for use in the present invention are compounds which are potent NK-1 receptor antagonists, i.e. compounds with an NK-1 receptor affinity (IC₅₀) of less than 10 nM.

Full descriptions of the preparation of the tachykinin receptor antagonists which may be employed in the present invention may be found in the references cited herein.

Unless otherwise defined herein, suitable alkyl groups include straight-chained and branched alkyl groups containing from 1 to 6 carbon atoms. Typical examples include methyl and ethyl groups, and straight-chained or branched propyl and butyl groups. Particular alkyl groups are methyl, ethyl, n-propyl, isopropyl, n-butyl, sec-butyl and tert-butyl.

Unless otherwise defined herein, suitable alkenyl groups include straight-chained and branched alkenyl groups containing from 2 to 6 carbon atoms. Typical examples include vinyl and allyl groups.

Unless otherwise defined herein, suitable alkynyl groups include straight-chained and branched alkynyl groups containing from 2 to 6 carbon atoms. Typical examples include ethynyl and propargyl groups.

Unless otherwise defined herein, suitable cycloalkyl groups include groups containing from 3 to 7 carbon atoms. Particular cycloalkyl groups are cyclopropyl and cyclohexyl.

Unless otherwise defined herein, suitable aryl groups include phenyl and naphthyl groups. A particular aryl-C₁₋₆alkyl, e.g. phenyl-C₁₋₆alkyl, group is benzyl.

Unless otherwise defined herein, suitable heteroaryl groups include pyridyl, quinolyl, isoquinolyl, pyridazinyl, pyrimidinyl, pyrazinyl, pyranyl, furyl, benzofuryl, thienyl, benzthienyl, imidazolyl, oxadiazolyl and thiadiazolyl groups.

The term “halogen” as used herein includes fluorine, chlorine, bromine and iodine. The compounds of use in this invention may have one or more asymmetric centres and can therefore exist as enantiomers and possibly as diastereoisomers. It is to be understood that the present invention relates to the use of all such isomers and mixtures thereof.

The above compounds are only illustrative of the neurokinin-1 (NK-1) antagonists which are useful in the methods of the instant invention. As this listing of compounds is not meant to be comprehensive, the methods of the present invention may employ any neurokinin-1 receptor antagonist, in particular a neurokinin-1 receptor antagonist which is orally active and long acting. Accordingly, the present invention is not strictly limited to any particular structural class of compound.

Certain of the above defined terms may occur more than once in the above formula and upon such occurrence each term shall be defined independently of the other. Similarly, the use of a particular variable within a noted structural formula is intended to be independent of the use of such variable within a different structural formula.

Full descriptions of the preparation of the tachykinin receptor antagonists which are employed in the present invention may be found in the references cited herein.

The identification of a compound as a tachykinin receptor antagonist, in particular, a neurokinin-1 receptor antagonist, and thus able to have utility in the present invention may be readily determined without undue experimentation by methodology well known in the art.

The compounds are useful in various pharmaceutically acceptable salt forms. The term “pharmaceutically acceptable salt” refers to those salt forms which would be apparent to the pharmaceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The term “pharmaceutically acceptable salts” also refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like, and basic ion exchange resins.

Suitable pharmaceutically acceptable salts of the compounds of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or solvent combination, or various combinations of solvents available Na-Z-L-2,3-diaminopropionic acid (Fluka) as a starting material is preferred.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below.

Abbreviations used in the description of the chemistry and in the Examples that follow are:

| | |
|---|---|
| Ac₂O | Acetic anhydride; |
| Boc | t-Butoxycarbonyl; |
| Bzl | Benzyl; |
| DABCO | 1,4-Diazabicyclo[2.2.2]octane; |
| or Dabco | |
| DBU | 1,8-diazabicyclo[5.4.0]undec-7-ene; |
| DCC | 1,3-Dicyclohexylcarbodiimide; |
| DIEA | Diisopropylethylamine; |
| DMAP | 4-Dimethylaminopyridine; |
| DME | 1,2-Dimethoxyethane; |
| DMF | Dimethylformamide; |
| DMSO | Dimethylsulfoxide; |
| DPPA | Diphenylphosphoryl azide; |
| EDC | 1-(3-dimethylaminopropyl)-3-ethyl-carbodiimide- |
| | hydrochloride; |
| HOAC | Acetic acid; |
| HOBT | 1-Hydroxybenzotriazole hydrate; |
| Et₃N | Triethylamine; |
| EtOAc | Ethyl acetate; |
| FAB | Fast atom bombardment; |
| HOOBT | 3-Hydroxy-1,2,2-benzotriazin-4(3H)-one; |
| HPLC | High-performance liquid chromatography; |
| MCPBA | m-Chloroperoxybenzoic acid; |
| MIBK | Methyl isobutyl ketone; |
| MsCl | Methanesulfonyl chloride; |
| MTBE | Methyl-t-butyl ether; |
| NaHMDS | Sodium bis(trimethylsilyl)amide; |
| NMP | 1-methyl-2-pyrrolidinone; |
| Py | Pyridine; |
| TEA | Triethanolamine; |
| TFA | Trifluoroacetic acid; |
| THF | Tetrahydrofuran; |
| TLC | Thin Layer Chromatography. |

The PSA conjugates of formulae I, III and V can be synthesized in accordance with Schemes 1-5, in addition to other standard manipulations such as ester hydrolysis, cleavage of protecting groups, etc., as may be known in the literature or exemplified in the experimental procedures.

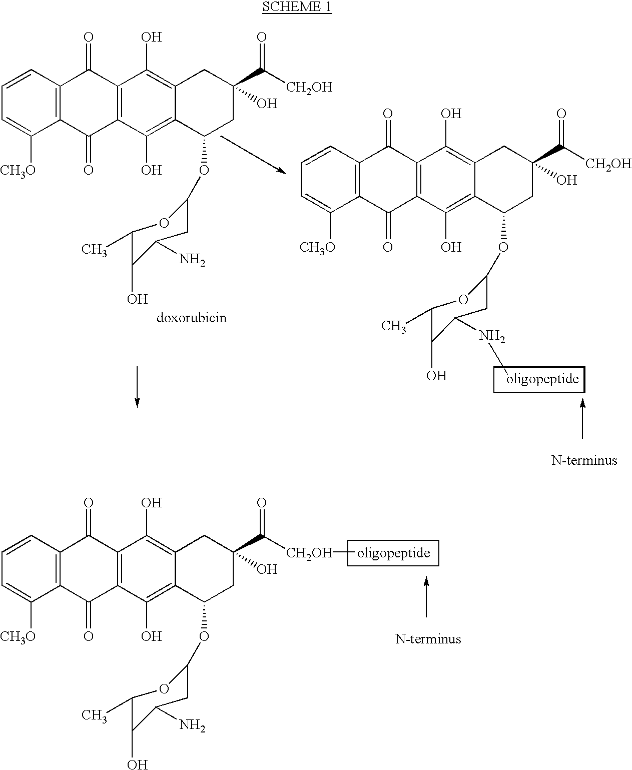

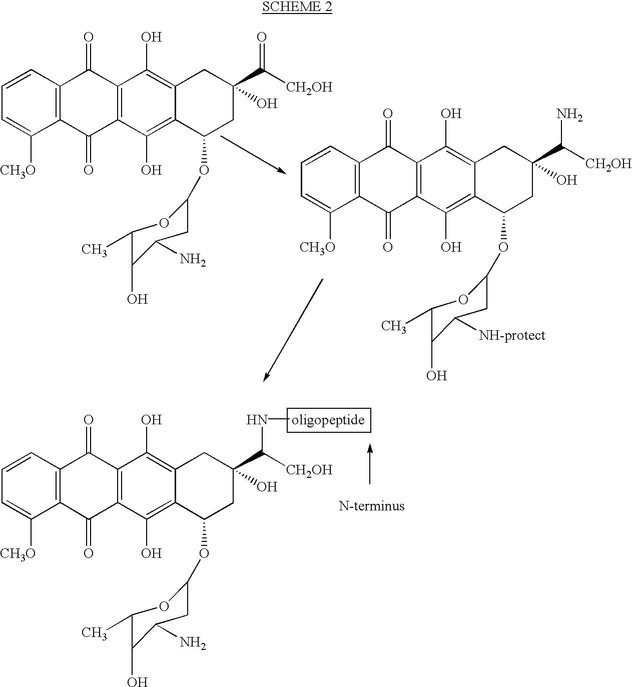

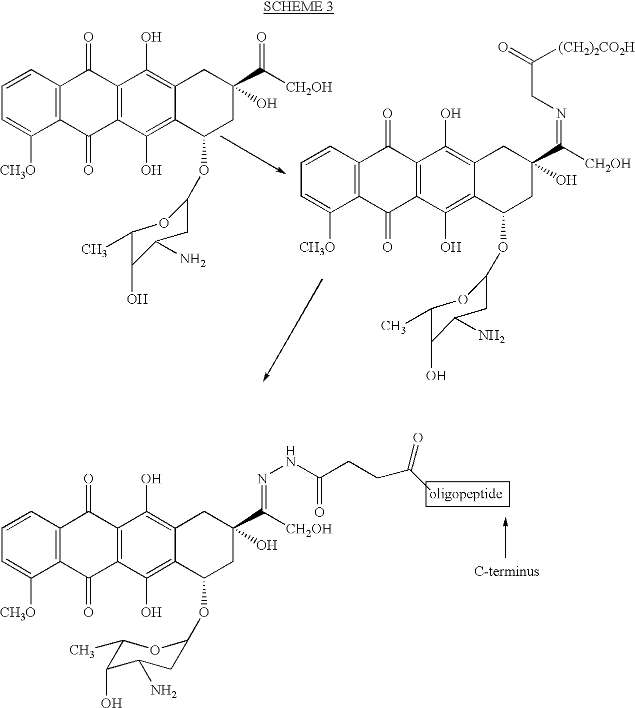

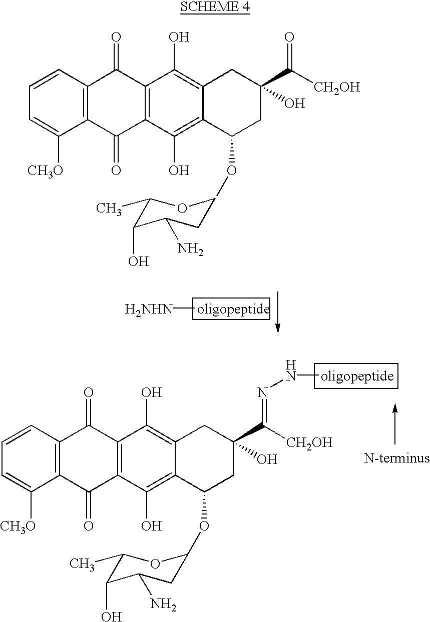

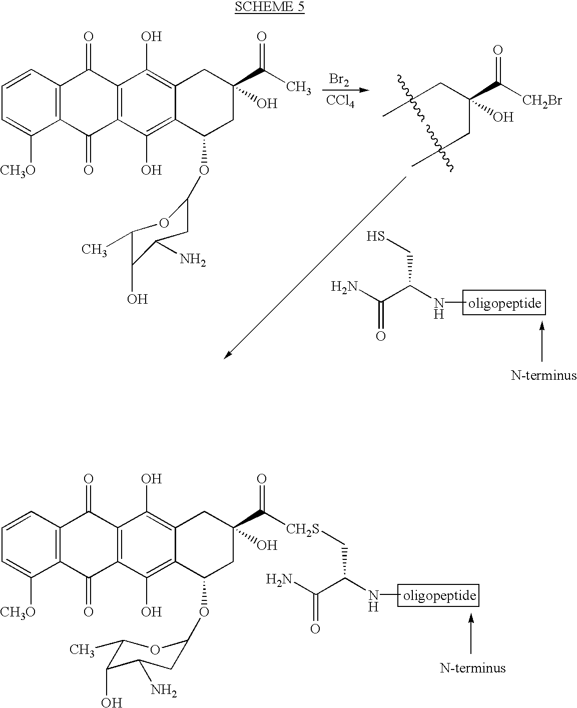

Scheme 6 illustrates preparation of conjugates utilized in the instant method of treatment wherein the oligopeptides are combined with the vinca alkaloid cytotoxic agent vinblastine. Attachment of the N-terminus of the oligopeptide to vinblastine is illustrated (S. P. Kandukuri et al. J. Med. Chem. 28:1079-1088 (1985)).

Scheme 7 illustrates preparation of conjugates of the oligopeptides of the instant invention and the vinca alkaloid cytotoxic agent vinblastine wherein the attachment of vinblastine is at the C-terminus of the oligopeptide. The use of the 1,3-diaminopropane linker is illustrative only; other spacer units between the carbonyl of vinblastine and the C-terminus of the oligopeptide are also envisioned. Furthermore, Scheme 7 illustrates a synthesis of conjugates wherein the C-4-position hydroxy moiety is reacetylated following the addition of the linker unit. Applicants have discovered that a desacetyl vinblastine conjugate is also useful in the instant methods. This conjugate may be prepared by eliminating the steps shown in Scheme 7 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the instant methods of treatment.

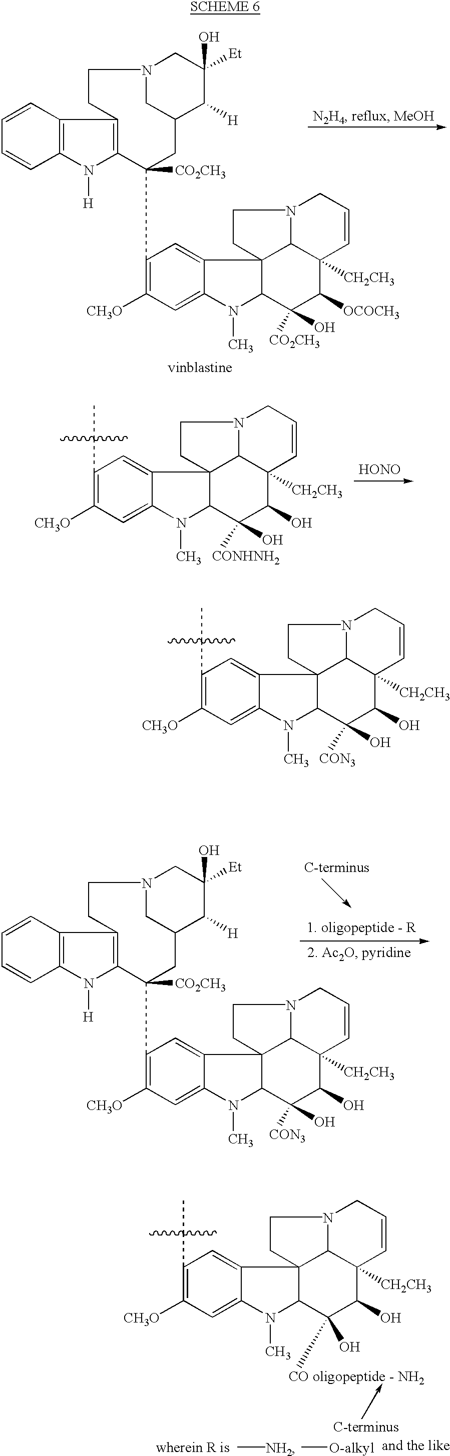

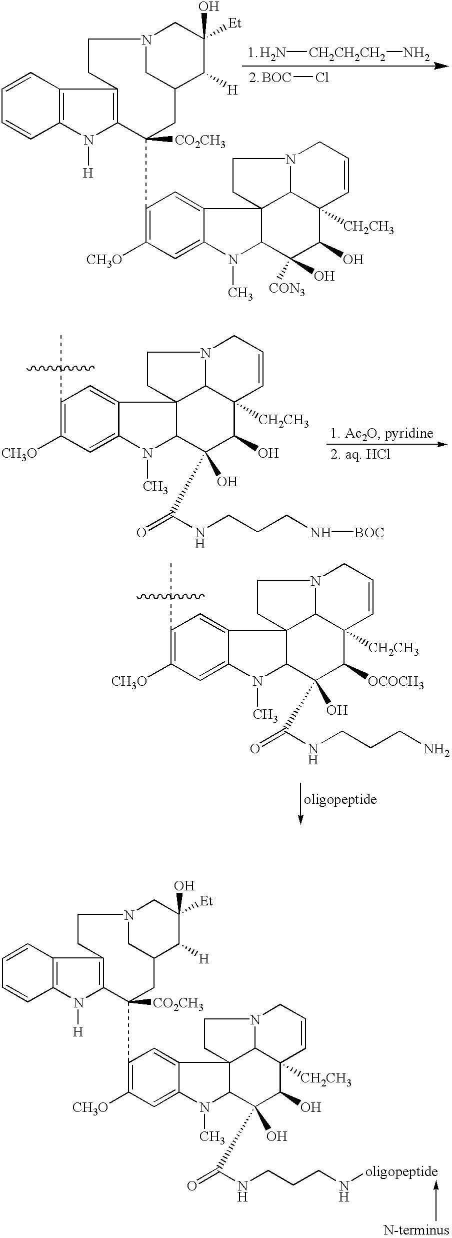

The PSA conjugates of formula III and V can be synthesized in accordance with Schemes 8-12, in addition to other standard manipulations such as ester hydrolysis, cleavage of protecting groups, etc., as may be known in the literature or exemplified in the experimental procedures.

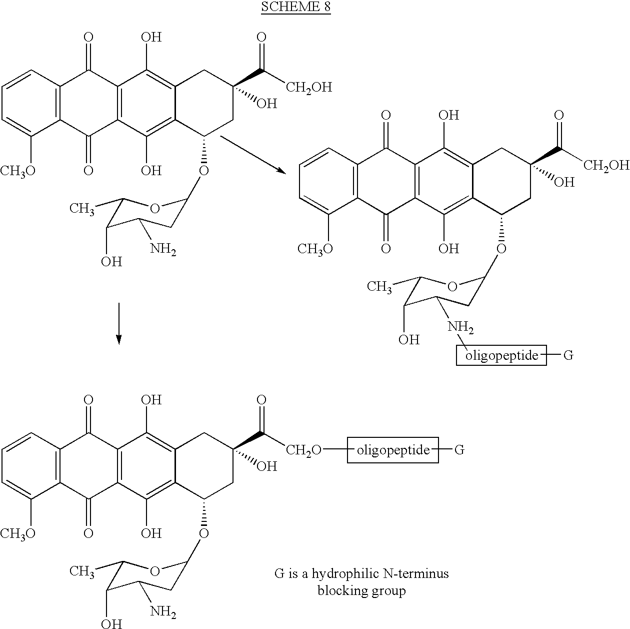

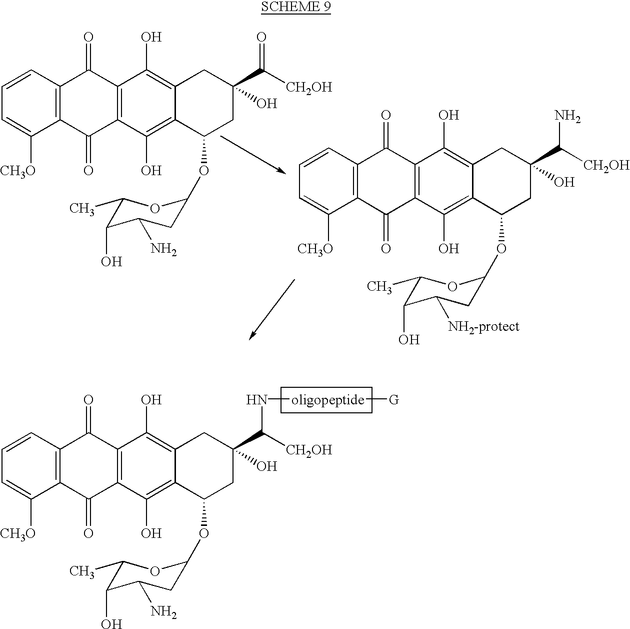

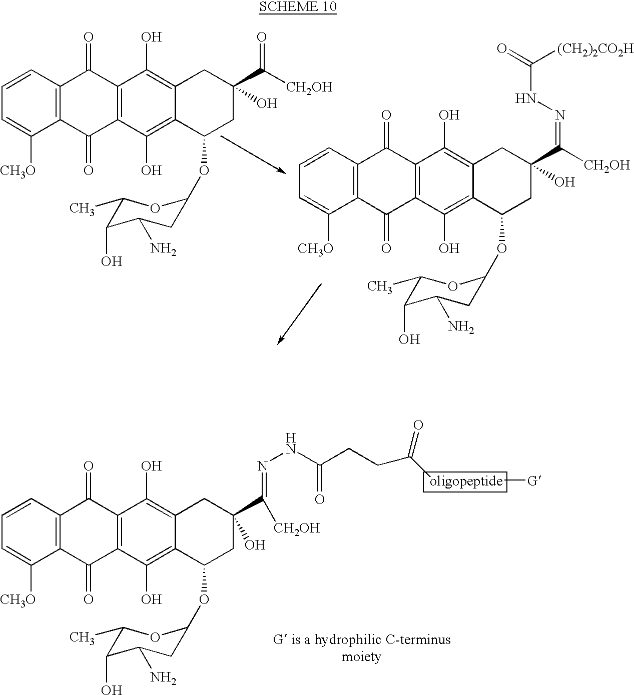

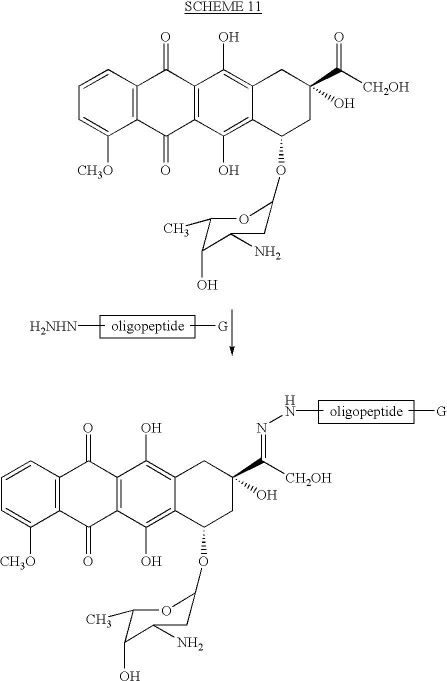

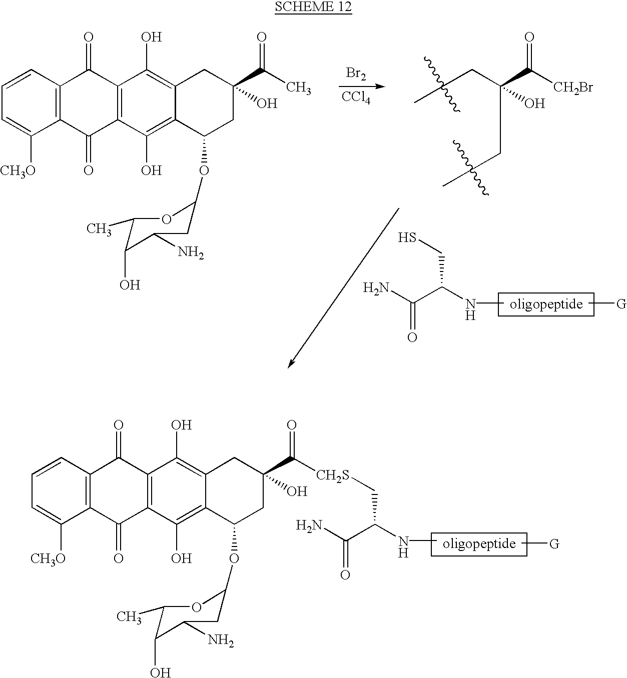

Scheme 13 illustrates preparation of PSA conjugates of the formula VI wherein the attachment of vinblastine is at the C-terminus of the oligopeptide. Furthermore, Scheme 13 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 13 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

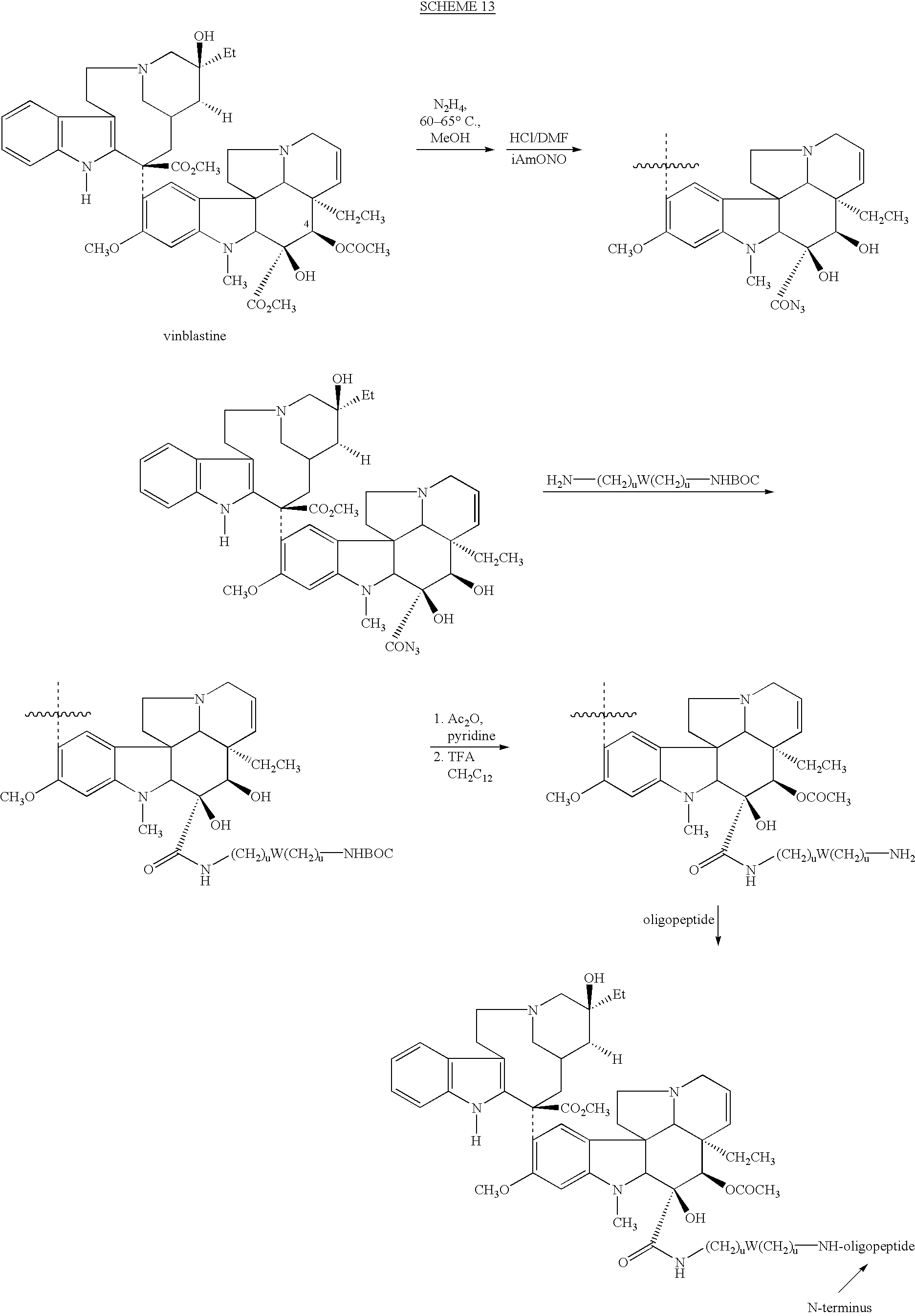

The PSA conjugates of formula VII can be synthesized in accordance with Schemes 14-15, in addition to other standard manipulations such as ester hydrolysis, cleavage of protecting groups, etc., as may be known in the literature or exemplified in the experimental procedures.

Reaction Scheme 14 illustrates preparation of conjugates of the oligopeptides of the instant invention and the vinca alkaloid cytotoxic agent vinblastine wherein the attachment of the oxygen of the 4-desacetylvinblastine is at the C-terminus of the oligopeptide. While other sequences of reactions may be useful in forming such conjugates, it has been found that initial attachment of a single amino acid to the 4-oxygen and subsequent attachment of the remaining oligopeptide sequence to that amino acid is a preferred method. It has also been found that 3,4-dihydro-3-hydroxy-4-oxo-1,2,3-benzotriazine (ODHBT) may be utilized in place of HOAt in the final coupling step.

Reaction Scheme 15 illustrates preparation of conjugates of the oligopeptides of the instant invention wherein a hydroxy alkanolyl acid is used as a linker between the vinca drug and the oligopeptide.

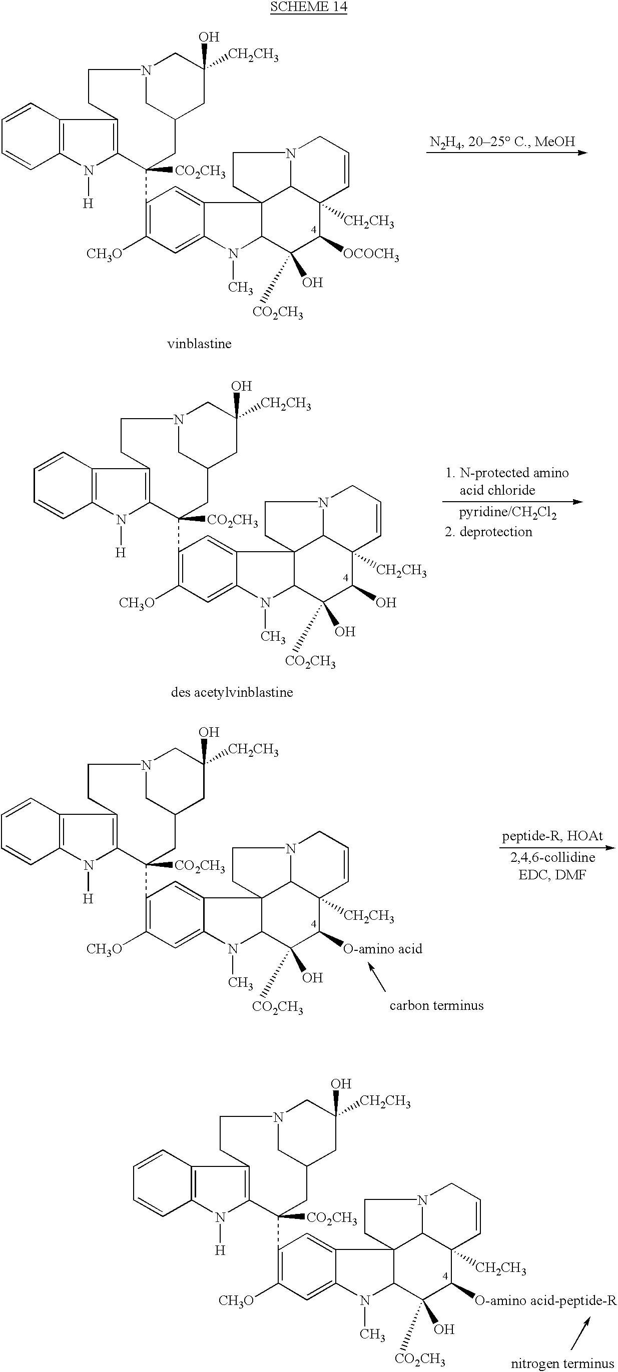

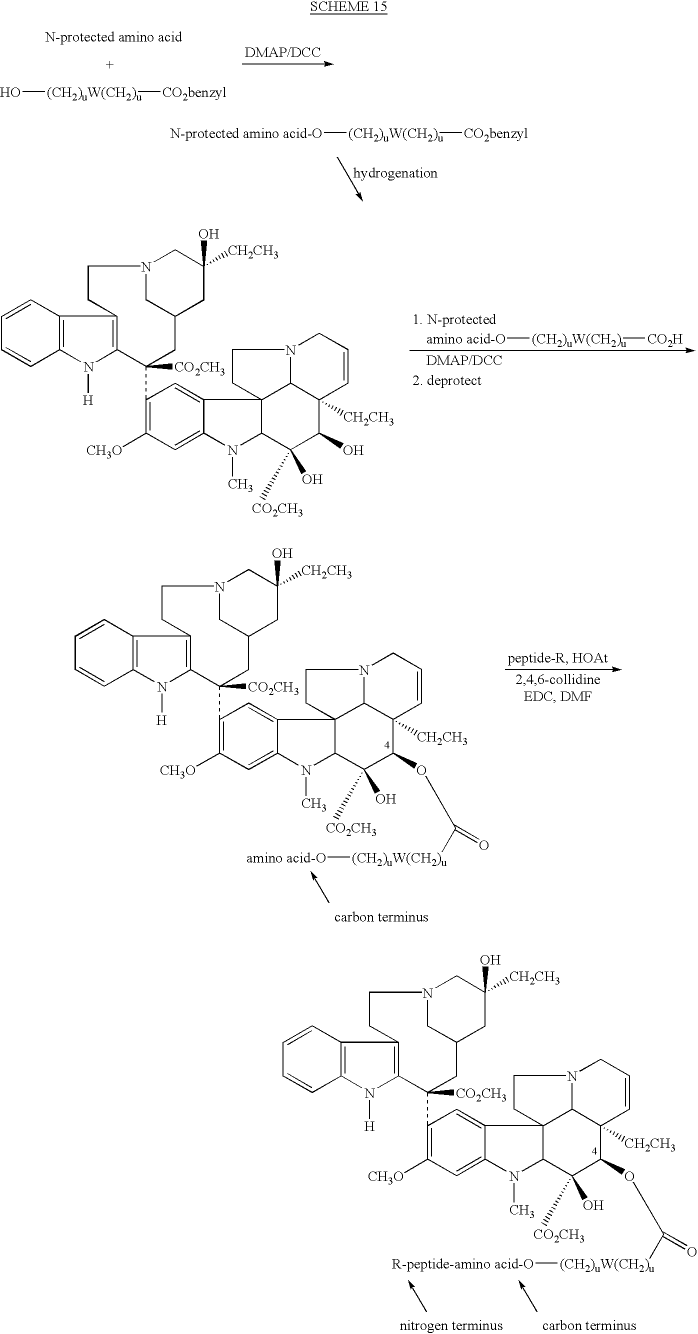

If it is desired to administer a PSA conjugate and a neurokinin-1 receptor simultaneously, a pharmaceutical composition comprising both agents may be preferable. Such a pharmaceutical composition of the instant invention may comprise one or more tachykinin receptor antagonist, in particular a neurokinin-1 receptor antagonist, and one or more PSA conjugates in combination. Also preferably, these agents are in combination with pharmaceutically acceptable carriers, excipients or diluents, according to standard pharmaceutical practice. The composition may be administered to mammals, preferably humans. The composition can be administered by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Preferably the compositions according to the present invention are in unit dosage forms such as tablets, pills, capsules, powders, granules, solutions or suspensions, or suppositories, for oral, parenteral or rectal administration, by inhalation or insufflation or administration by trans-dermal patches or by buccal cavity absorption wafers.

For preparing solid compositions such as tablets, the principal active ingredient(s) is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Preferred compositions for administration by injection include those comprising a NK-1 receptor antagonist and a PSA conjugate as the active ingredients, in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 μm, particularly 0.1 and 0.5 μm, and have a pH in the range of 5.5 to 8.0.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Compositions of the present invention may also be presented for administration in the form of trans-dermal patches using conventional technology. The compositions may also be administered via the buccal cavity using, for example, absorption wafers.

Compositions in the form of tablets, pills, capsules or wafers for oral administration are particularly preferred.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions useful in the instant methods of treatment may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of a suppositories for rectal administration of the drug. These compositions can be prepared by mixing the instant composition with a suitable non-irritating excipient which is solid at ordinary temperatures but liquid at the rectal temperature and will therefore melt in the rectum to release the composition. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the combination of tachykinin receptor antagonist(s) and PSA conjugate(s) are employed. (For purposes of this application, topical application shall include mouth washes and gargles.)

The compositions useful in the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The composition of a tachykinin receptor antagonist, a PSA conjugate(s), or a combination thereof useful in the instant methods of treatment may also be co-administered with other well known therapeutic agents that are selected for their particular usefulness against the condition that is being treated.

The instant method of treatment may also be combined with surgical treatment (such as surgical removal of tumor and/or prostatic tissue) where appropriate. The above descriptions of pharmaceutical compositions are also applicable to pharmaceutical compositions of the individual agents if they are administered separately according to the invention.

If formulated as a fixed dose, the compositions useful in the instant invention employ the tachykinin receptor antagonist(s) and the PSA conjugate(s) within the dosage ranges described below.

When compositions according to this invention are administered into a human subject, the daily dosage will normally be determined by the prescribing physician with the dosage generally varying according to the age, weight, and response of the individual patient, as well as the severity of the patient's symptoms.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease or disorder, the patient's weight, special diets then being followed by a patient, concurrent medication, the intrinsic tachykinin receptor antagonist activity of the compound, the bioavailability upon oral administration of the compound and other factors which those skilled in the art will recognize.

In the treatment of a condition in accordance with the present invention, an appropriate dosage level will generally be about 0.01 μg to 50 mg per kg patient body weight per day which may be administered in single or multiple doses. Preferably, the dosage level will be about 0.1 μg to about 25 mg/kg per day; more preferably about 0.5 μg to about 10 mg/kg per day. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 300 mg active ingredient. A minimum dosage level for the NK-1 receptor antagonist is generally about 5 mg per day, preferably about 10 mg per day and especially about 20 mg per day. A maximum dosage level for the NK-1 receptor antagonist is generally about 1 500 mg per day, preferably about 1000 mg per day and especially about 500 mg per day. Administration of the PSA conjugate occurs in an amount between about 10 mg/m² of body surface area to about 4 g/m² of body surface per day, preferably between about 50 mg/m² of body surface to about 3 g/m² of body surface per day.

It will be appreciated that the amount of the composition of the instant invention required for use in treating cancer in a patient will vary not only with the particular compounds or compositions selected but also with the route of administration, the nature of the condition being treated, and the age and condition of the patient, and will ultimately be at the discretion of the patient's physician or pharmacist. The length of time during which the instant composition will be given varies on an individual basis.

It will be appreciated by those skilled in the art that reference herein to treatment extends to prophylaxis (prevention) as well as the treatment of the noted diseases/disorders and symptoms. Because the specific diagnosis of chronic nonbacterial prostatitis or prostatodynia in a particular patient may be difficult, the patient may benefit from the prophylactic administration of a subject compound in accordance with the present invention.

Examples are provided for the purpose of further illustration only and are not intended to be limitations on the disclosed invention.

The starting materials and reagents for the subject processes are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The following examples are provided for the purpose of further illustration only and are not intended to be limitations on the disclosed invention.

### EXAMPLE 1

4-Benzyl-2-hydroxy-1,4-oxazin-3-one

| Materials | MW | Density | Amount | | mol | Equiv. |
|---|---|---|---|---|---|---|
| N-Benzylethanolamine | 151.21 | 1.065 | 7.80 | kg | 49.5 | 1.0 |
| (96%) | | | | | (assay) | |
| Glyoxylic acid | 74.04 | 1.342 | 12.60 | L | 114.2 | 2.31 |
| (50% in water) | | | | | | |
| Tetrahydrofuran | 72.11 | 0.889 | 27.0 | L | — | — |
| 4-Benzyl-2-hydroxy- | 207.23 | — | 0.252 | kg | 1.24 | 0.025 |
| 1,4-oxazin-3-one seed | | | | | | |
| Water | 18.0 | 1.00 | 63.0 | L | — | — |

A solution of THF (27.0 L) and 50% aqueous glyoxylic acid (12.6 L; 16.9 kg) was heated to reflux and N-benzylethanolamine (7.8 kg) was added over 45 min. The resulting mixture was refluxed for 21 h. Then the THF was distilled under atmospheric pressure while maintaining a constant volume by simultaneous addition of water (27 L). Upon completion of the distillation (<8 vol % of THF in batch) the mixture was cooled from approximately 95-100 to 79-81° C. and was optionally seeded with 4-benzyl-2-hydroxy-1,4-oxazin-3-one (250 g). Upon further cooling to room temperature the product crystallized. Crystalline 4-benzyl-2-hydroxy-1,4-oxazin-3-one was filtered, washed with water and then dried in a vacuum oven at about 60° C. under a stream of N₂ (72-76% yield); m.p. 134° C.

### EXAMPLE 2

3,5-Bis(trifluoromethyl)bromobenzene

| Materials | MW | Density | Amount | | Mmol | Equiv. |
|---|---|---|---|---|---|---|
| 1,3-Bis(trifluoro- | 214.1 | 1.38 | 107 | g | 500 | 1.0 |
| methyl) benzene | | | | | | |
| 96% H₂SO₄ | | | 142 | mL | | |
| Glacial HOAc | | | 22 | mL | | |
| 1,3-Dibromo-5,5- | 285.93 | | 77.25 | g | 270 | 1.08 |
| dimethylhydantoin | | | | | | (Br⁺) |
| 5N Aq NaOH | | | 75 | mL | | |

A vigorously stirred solution of 1,3-bis(trifluoromethyl)benzene (107 g) in a mixture of glacial acetic acid (22 mL) and concentrated sulfuric acid (142 mL) was added 1,3-dibromo-5,5-dimethylhydantoin (77.25 g) at 25° C. The exothermic reaction raised the temperature to approximately 40° C. After aging at 45° C. for 4.5 hours, the mxture was cooled to approximately 0° C. and poured into cold water (250 mL). After washing with 5N NaOH (75 mL) the organic layer contained 137 g of the desired 3,5-bis(trifluoromethyl)-1-bromobenzene by assay (94% yield). This product was used in the next step without further purification.

### EXAMPLE 3

1-(3,5-Bis(trifluoromethyl)phenyl)ethan-1-one

| Materials | MW | Density | | Amount | | Mmol | Equiv |
|---|---|---|---|---|---|---|---|
| 3,5-Bis(trifluoro- | 293.03 | 1.699 | g/L | 29.3 | g | 98.0 | 1.0 |
| methyl)bromo- | | | | | | | |
| benzene | | | | | | | |
| Magnesium | 24.3 | | | 5.10 | g | | 2.1 |
| granules, 20 mesh | | | | | | | |
| Acetic Anhydride | 102.1 | 1.08 | g/L | 40 | mL | 423 | 4.5 |
| THF | | | | 260 | mL | | |
| (KF = 60 μg/mL) | | | | | | | |
| MTBE | | | | 650 | mL | | |
| Water | | | | 300 | mL | | |
| 50% NaOH | | | | 40 | mL | | |

A solution of 3,5-Bis(trifluoromethyl)bromobenzene (29.3 g) in 30 mL of THF was added to a mixture of magnesium granules (5.10 g) in THF (200 mL) heated at reflux (the reaction was initiated with approximately 5 mL of the bromide solution; the remainder was added slowly over 1 hour). The mixture was aged for 30 minutes at reflux, cooled to room temperature and added over 1 hour to a solution of acetic anhydride (40 mL) in THF (40 mL) maintained at −15° C. The resulting dark brown mixture was warmed to 10° C. in a water bath, and water (300 mL) was added. The pH of the vigorously stirred biphasic mixture was adjusted to 8.0 using 50% NaOH. MTBE (300 mL) was added, the layers were separated and the aqueous layer was further extracted with MTBE (3×150 mL). The organic layers were combined and concentrated in vacuo (bath at 30-35° C.; 50-80 torr). The concentrate was then distilled at atmospheric pressure to provide the pure product (20.7 g; 82% yield) with a boiling point of 187-189° C.

### EXAMPLE 4

(R)-1-(3,5-Bis(trifluoromethyl)phenyl)ethan-1-ol

| | | | | |
|---|---|---|---|---|
| 1-(3,5-Bis(trifluoromethyl)- | 256.15 | 3.9 | 1 | Kg |
| phenyl) ethan-1-one | | | | |
| (Cp*RhCl₂)₂ | 618.08 | 0.01 | 6 | g |
| (Cp* = Pentamethylcyclopentadienyl) | | | | |
| (S,R)-cis-Aminoindanol | 149.20 | 0.02 | 3.0 | g |
| NaOH | 5 N (H₂O) | 0.05 | 9 | mL |
| IPA | | | 7 | L |
| HCl | 1 N (H₂O) | | 7 | L |
| Heptane | | | 7 | L |
| 1,4-diazabicyclo[2.2.2]octane | 112.18 | 2.2 | 240 | g |
| (DABCO) | | | | |

A solution of [Cp*RhCl₂]₂ (Cp*=pentamethylcyclopentadienyl; 6.0 g), (1S,2R)-cis-1-amino-2-indanol (3.0 g) and 1-(3,5-Bis(trifluoromethyl)phenyl)ethan-1-one (1.0 kg) in 2-propanol (7 L) was stirred for 30 minutes and thoroughly degassed under vacuum. Then 5 M sodium hydroxide (9 mL) was added and the mixture was aged for 3-4 hours to achieve complete conversion of the starting material. The reaction mixture was poured into 1 N HCl (7 L) and extracted with heptane (2×3.5 L). The combined organic layers were washed with brine (5 L) and 1,4-diazabicyclo [2.2.2]-octane (240 g) was added. The solution was concentrated to approximately 4 ml/g of alcohol (KF<200 μg/mL; 2-propanol <5 vol %). The mixture was seeded at 40° C., allowed to cool to room temperature to form a seedbed and then cooled to 0° C. The crystalline product was filtered, washed with cold heptane and dried to provide the DABCO complex (70% yield; e.e.>99%).

### EXAMPLE 5

(R)-1-(3,5-Bis(trifluoromethyl)phenyl)ethan-1-ol

| Materials | MW | Mol | Amt | |
|---|---|---|---|---|
| 1-(3,5-Bis(trifluoromethyl)- | 256.15 | 11.7 | 3 | Kg |
| phenyl)ethan-1-one | | | | |
| [RuCl₂(p-cymene)]₂ | 612.40 | 0.03 | 18.4 | g |
| (Cym = p-cymene (4-isopropyltoluene)) | | | | |
| (S,R)-cis-Aminoindanol | 149.20 | 0.06 | 9.0 | g |
| NaOH | 5 N (H₂O) | 0.14 | 28 | mL |
| IPA | | | 21 | L |
| HCl | 1 N (H₂O) | 21 | L | |
| Heptane | | | 21 | L |
| 1,4-Diazabicyclo[2.2.2]octane | 112.18 | ˜6.6 | ˜740 | g |
| (DABCO) | | | | |

A solution of [RuCl₂(p-cymene)]₂ (18.4 g), (1S,2R)-cis-1-amino-2-indanol (9.0 g) and 1-(3,5-bis(trifluoromethyl)phenyl)ethan-1-one (3 kg) in 2-propanol (21 L) was stirred for 30 minutes and thoroughly degassed under vacuum. Then 5 M sodium hydroxide (28 mL) was added and the mixture was aged for 4-6 hours to achieve complete conversion of the starting material. The reaction mixture was poured into 1 N HCl (21 L) and extracted with heptane (2×10.5 L). The combined organic layers were washed with brine and 1,4-diazabicyclo[2.2.2]octane (740 g) was added. The solution was concentrated to approximately 4 mL/g of alcohol (KF<200 μg/mL; 2-propanol <5 vol %). The mixture was seeded at 40° C., allowed to cool to room temperature to form a seedbed and then cooled to 0° C. The crystalline product was filtered, washed with cold heptane and dried to provide the DABCO complex (75-80% yield; e.e.>99%).

### EXAMPLE 6

(2R, 2-alpha-R)-4-Benzyl-2-[1-[3,5-bis(trifluoromethyl)phenyl]ethoxy-1,4-oxazin-3-one (Process 1)

| | | | | | | mol |
|---|---|---|---|---|---|---|
| Materials | Kg | L | mol | MW | density | % |
| 4-Benzyl-2-hydroxy-1,4- | 2.14 | | 10.3 | 207.2 | | 100 |
| oxazin-3-one | | | | | | |
| Trifluoroacetic anhydride | 2.16 | 1.46 | 10.3 | 210.0 | 1.487 | 100 |
| (R)-(3,5-bis(trifluoro- | 5.11 | 5.13 | 9.80 | 258.2 | 0.996 | 95 |
| methyl)phenyl) ethan-2- | | | | | | |
| ol (49.5 wt % solution in | | | | | | |
| acetonitrile) | | | | | | |
| Boron trifluoride etherate | 0.73 | 0.65 | 5.14 | 141.9 | 1.120 | 50 |
| 5N NaOH (aq) | 7.60 | 38.0 | | | 370 | |
| 3,7-Dimethyloctan-3-ol | 4.90 | 5.93 | 31.0 | 158.3 | 0.826 | 300 |
| Potassium t-butoxide | 0.75 | | | 112.2 | | 65 |
| (solid) | | | | | | |
| Acetic acid (neat) | 0.62 | 0.59 | 10.3 | 60.05 | 1.049 | 100 |
| Acetonitrile | | 5.3 | | | | |
| Heptane | | 27 | | | | |
| 5% Sodium bicarbonate | | 5 | | | | |
| (aq) | | | | | | |
| Water | | 23 | | | | |

Trifluoroacetic anhydride (2.16 kg) was added over 10 minutes to a dry (KF<100 μg/mL) slurry of lactam lactol (2.14 kg) in acetonitrile (5 L) cooled at 5° C. The temperature rose from 5 to 30° C. and the solids dissolved. The solution was aged for 1 hour between 17-25° C. before a concentrated solution of (R)-(3,5-bis(trifluoromethyl)phenyl)ethan-2-ol in acetonitrile (5.11 kg of solution containing 2.53 kg of alcohol) was added followed by BF₃ etherate (0.65L). The temperature rose from 17 to 27° C. and the mixture was aged for 4 hours before 5 M NaOH (7.6 L) was added slowly while maintaining the temperature below 27° C. followed by 3,7-dimethyloctan-3-ol (5.9 L). The resulting mixture was distilled at atmospheric pressure until the vapor temperature reached 92° C. and most of the acetonitrile was distilled off. Water (5 L) and heptane (8 L) were added and the mixture was warmed to 45° C. The organic layer was separated, washed with water (13 L) at 45-50° C. and then diluted with heptane (16 L). The solution was dried via an azeotropic distillation until KF<130 μg/mL (6 L of distillate collected; 3 L of fresh heptane added). The solution was cooled to room temperature and seeded with the (R,R) diastereomer (50 mg). Upon formation of a seedbed the slurry was cooled to −10° C. and potassium tert-butoxide (752 g) was added in one portion. The mixture was aged between −12 and −7° C. for 8.5 hours when virtually all of the undesired diastereomer had been converted to the desired (R,R) diastereomer according to HPLC analysis. Acetic acid (0.59 L) was added followed by a 5% NaHCO₃ in water solution (5 L). The biphasic mixture was warmed to 45-50° C. The organic layer was separated, washed with water (5 L) at 45-50° C. and concentrated via distillation at atmospheric pressure to a total volume of 24 L (12 L of distillate collected). Upon cooling to 35° C. a seedbed formed. The slurry was cooled to −10° C. and then filtered. The solids were washed with cold heptane (4.5 L) and dried in vacuo to provide the pure product (3.66 kg; 83% overall yield).

### EXAMPLE 7

(2R, 2-alpha-R)-4-Benzyl-2-[1-[3,5-bis(trifluoromethyl)phenyl]ethoxy-1,4-oxazin-3-one (Process 2)

| Materials | Kg | L | mol | MW | density | mol % |
|---|---|---|---|---|---|---|
| 4-Benzyl-2-hydroxy- | 2.03 | | 9.80 | 207.2 | | 100 |
| 1,4-oxazin-3-one | | | | | | |
| Trifluoroacetic | 2.06 | 1.38 | 10.3 | 210.0 | 1.487 | 100 |
| anhydride | | | | | | |
| (R)-(3,5-bis(trifluoro- | 4.85 | 4.87 | 9.30 | 258.2 | 0.996 | 95 |
| methyl)phenyl) ethan- | | | | | | |
| 2-ol (49.5 wt % solu- | | | | | | |
| tion inacetonitrile) | | | | | | |
| Boron trifluoride | 0.69 | 0.62 | 4.86 | 141.9 | 1.120 | 50 |
| etherate | | | | | | |
| 5N NaOH (aq) | | 8.1 | 40.5 | | | 410 |
| 3,7-Dimethyloctan- | | 1.68 | 2.03 | 10.6 | 158.3 | 0.826 |
| 3-ol | | | | | | |
| Potassium 3,7-di- | 1.09 | 1.36 | 2.70 | 196.4 | 0.803 | 28 |
| methyloct-3-oxide | | | | | | |
| (48.7 wt % in heptane, | | | | | | |
| 1.99 M) | | | | | | |
| Acetic acid (neat) | 0.28 | 0.29 | 4.66 | 60.05 | 1.049 | 47 |
| Acetonitrile | | 4.8 | | | | |
| Heptane | | 21 | | | | |
| 5% Sodium | | 4.1 | | | | |
| bicarbonate (aq) | | | | | | |
| Water | | 20.4 | | | | |

Trifluoroacetic anhydride (2.056 kg) was added over 10 minutes to a dry (KF<140 μg/mL) slurry of lactam lactol (2.03 kg) in acetonitrile (4.8 L) cooled at 5° C. The temperature rose from 5 to 34° C. and the solids dissolved. The solution was aged for 1 hour between 17-25° C. before a concentrated solution of (R)-(3,5-bis (trifluoromethyl)phenyl)ethan-2-ol in acetonitrile (4.85 kg of solution containing 2.40 kg of alcohol) was added followed by BF₃ etherate (0.62 L). The temperature rose from 17 to 28° C. and the mixture was aged for 2 hours before 5 M NaOH (8.1 L) was added slowly while maintaining the temperature below 27° C., followed by 3,7-dimethyloctan-3-ol (2.0 L). The resulting mixture was distilled at atmospheric pressure until the vapor temperature reached 92° C. and most of the acetonitrile was distilled off (8.1 L of distillate collected). Water (4.1 L) and heptane (12.2 L) were added and the mixture was warmed to 45° C. The organic layer was separated, washed with water (12.2 L) at 45-50° C. and then diluted with heptane (6 L). The solution was dried via an azeotropic distillation until KF<130 μg/mL (7.8 L of distillate collected). The solution was cooled to room temperature and seeded with the (R,R) diastereomer (50 mg). Upon formation of a seedbed the slurry was cooled to −11° C. and potassium 3,7-dimethyloct-3-oxide (1.09 kg; 48.7 wt % solution in heptane) was over 10 minutes. The mixture was aged between −12 and −7° C. for 5 hours during which virtually all of the undesired diastereomer had been converted to the desired (R,R) diastereomer according to HPLC analysis. Acetic acid (0.28 L) was added followed by a 5% NaHCO₃ in water solution (4.1 L). The biphasic mixture was warmed to 45-50° C. The organic layer was separated, washed with water (4.1 L) at 45-50° C. and concentrated via distillation at atmospheric pressure to a total volume of 16 L (4.1 L of distillate collected). The solution was seeded at 45° C. and then allowed to cool to room temperature. The slurry was cooled to 5° C., aged for 1.5 hours and then filtered. The solids were washed with cold heptane (3.0 L) and dried in vacuo to provide the pure product (3.51 kg; 84% overall yield).

### EXAMPLE 8

(2R, 2-alpha-R, 3a)-2-[1-[3,5-bis(trifluoromethyl)phenyl]ethoxy-3-(4-fluorophenyl)-1,4-oxazine (Process 1)

A solution of (2R, 2-alpha-R)-4-benzyl-2-[1-[3,5-bis(trifluoromethyl)-phenyl]ethoxy-1,4-oxazin-3-one (4.80 g) in toluene (27 mL) was cooled to −6° C. and a solution of 4-fluorophenylmagnesium bromide in THF (27 mL; 0.93 M) was added slowly such that T<0° C. The resulting clear solution was aged for 1 hour and then quenched into aqueous citric acid (10 wt %; 27 mL). Toluene (27 mL) was added and the organic layer was separated and washed with 0.5 M sodium bicarbonate solution and water (25 mL each). The solution was partially concentrated to a total volume of approximately 20 mL and then diluted with methanol to a total volume of 90 mL. 4-Toluenesulfonic acid monohydrate (1.95 g) and 10% Pd/C catalyst (950 mg) were added and the mixture was hydrogenated under 5 psi of hydrogen at RT for 4 hours. The catalyst was filtered and washed with additional methanol (90 mL). The combined filtrates were concentrated to dryness. The residue was dissolved in 60 mL of hot toluene. Upon slow cooling crystals were formed. After cooling to room temperature, heptane (60 mL) was added. After filtration and drying, (2R, 2-alpha-R, 3a)-2-[1-[3,5-bis(trifluoromethyl)phenyl]ethoxy-3-(4-fluorophenyl)-1,4-oxazine as the tosylate salt was obtained in 89% overall yield.

### EXAMPLE 9

(2R, 2-alpha-R, 3a)-2-[1-[3,5-bis(trifluoromethyl)phenyl]ethoxy-3-(4-fluorophenyl)-1,4-oxazine (Process 2)

A solution of 4-fluorophenylmagnesium bromide in THF (150 mL; 0.93 M) was slowly added to a solution of (2R, 2-alpha-R)-4-benzyl-2-[1-[3,5-bis (trifluoromethyl)phenyl]ethoxy-1,4-oxazin-3-one (49.5 g) in THF (50 mL) between 20 and 25° C. The resulting clear solution was aged for 30 minutes and then added slowly into cold methanol (100 mL) such that T<20° C. A solution of 4-toluene-sulfonic acid monohydrate (42.1 g) in methanol (50 mL) was added to the slurry followed by the 5% Pd/C catalyst (16.5 g; 55 wt % wet). The resulting mixture was hydrogenated under 5 psi of hydrogen at room temperature for 3 hours. The catalyst was filtered and washed with methanol (100 mL). The combined filtrates were concentrated via distillation at atmospheric pressure to a total volume of approximately 350 mL. The distillation was continued while keeping the volume constant at 350 mL via slow addition of 4-methyl-2-pentanone (methyl-isobutyl ketone; MIBK; 450 mL). Upon completion of the distillation, the resulting slurry was allowed to cool to 20-30° C. and washed with 500 mL of a solution of trisodium citrate dihydrate (10 wt %) and sodium bicarbonate (1.0 M) in water. Concentrated hydrochloric acid (11.9 g; 37.3 wt %) was added to the organic layer and the clear solution was concentrated under atmospheric pressure to a total volume of approximately 180 mL. The resulting product slurry was cooled from 118° C. to 5° C. and filtered. The solids were washed with methyl-isobutyl ketone (MIBK) and dried in vacuo at 40° C. to afford 46.4 g of (2R, 2-alpha-R, 3a)-2-[1-[3,5-bis(trifluoromethyl)phenyl]ethoxy-3-(4-fluorophenyl)-1,4-oxazine as the hydrochloride salt in 87% overall yield.

### EXAMPLE 10

(2R, 2-alpha-R, 3a)]-5-[[2-[1-[3,5-bis(trifluoromethyl)phenyl]ethoxy]-3-(4-fluorophenyl)-4-morpholinyl]methyl]-12-dihydro-3H-1,2,4-triazol-3-one Powdered potassium carbonate (682 g, 4.93 moles) is added to a solution of (2R, 2-alpha-R, 3a)-2-[1-[3,5-bis(trifluoromethyl)phenyl]ethoxy-3-(4-fluorophenyl)-1,4-oxazine hydrochloride (2.06 moles), N-methylcarboxy-2-chloroacetamidrazone (375 g, 2.26 moles), and dimethylformamide (10 L). The reaction was maintained between 15 and 25° C. and aged for 2.5 hours. The batch is diluted with 1:1 hexane:methyl-t-butyl ether (10 L) and 10.9% aqueous ammonium chloride (11 L). The phases were partitioned and the aqueous phase is back extracted with 1:1 hexane:methyl-t-butyl ether (2×8 L), followed by 1:2 hexane:methyl-t-butyl ether (8 L). The combined organic phases are washed with water (2×15 L), then vacuum concentrated. The resulting material is dissolved in xylenes (20 L) and heated to reflux (137° C.). The solution is maintained at reflux for 3 hours, then cooled to ambient temperature, whereupon (2R, 2-alpha-R, 3a)]-5-[[2-[1-[3,5-bis(trifluoromethyl)phenyl]ethoxy]-3-(4-fluorophenyl)-4-morpholinyl]methyl]-1,2-dihydro-3H-1,2,4-triazol-3-one crystallizes. The batch is aged overnight, then filtered. The filter cake is washed with xylenes (2 L), then hexanes (2×2 L), then dried under vacuum at 30° C. for three days to yield (2R, 2-alpha-R, 3a)]-5-[[2-[1-[3,5-bis(trifluoromethyl)phenyl]ethoxy]-3-(4-fluorophenyl)-4-morpholinyl]methyl]-1,2-dihydro-3H-1,2,4-triazol-3-one.

Alternatively, the title product may be prepared as follows from the (2R, 2-alpha-R, 3a)-2-[1-[3,5-bis(trifluoromethyl)phenyl]ethoxy-3-(4-fluorophenyl)-1,4-oxazine tosylate salt, (1.90 Kg, 3.12 mol); N-methylcarboxyl-2-chloroacetamidrazone (516.3 g, 3.12 mol); K₂CO₃ (1.08 kg, 2.5 eq.); and DMSO (15.6 L). To a suspension of amine salt and powder K₂CO₃ in DMSO (7.8L) at 20° C. is added a solution of N-methylcarboxyl-2-chloroacetamidrazone in DMSO (7.8L). The first half of the solution is added quickly, (with slight cooling with ice water bath) then the remaining half is added over a period of 1 hour. After the addition, the reaction is checked by LC, and the reaction is quenched with cold water (15L) and methyl-t-butyl ether (MTBE) (30L) solution. The organic layr is separated, and washed with water, sat. NaHCO₃, brine, and water (20L/each) respectively. The aqueous layers is back extracted with additional MTBE (15L). The combined MTBE solution is concentrated to an oil. The resulting crude product is dissolved in xylene (25L) and diisopropylethylamine (6.25 L) and is heated to reflux (˜135° C.) and the reaction is monitored by LC. The reaction takes 4-6 hours to complete, the reaction solution is cooled down to room temperature overnight and filter to get the title product (expect 1.33 kg, ˜80%, typically purity 98.5A %).

The resulting crude product is dissolved in hot methanol (13.3L), added charcoal 133 g, then filtered and the charcoal is washed with hot methanol (3.3L). The methanol solution is cooled down to room temperature, then water (7L) is added dropwise. After being stirred at room temperature for 2 hours, the suspension is filtered to isolate purified product as a white crystalline compound (expect 1.20 kg, 90% recovery, typical purity, 99.5A %).

### EXAMPLE 11

Preparation of [N-Ac-(4-trans-L-Hyp)]-Ala-Ser-Chg-Gln-Ser-Leu-Dox

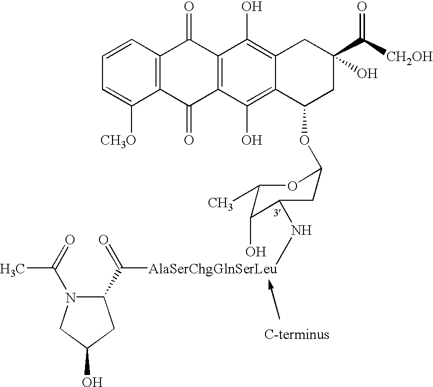

Step A: [N-Ac-(4-trans-L-Hyp(Bzl))]-Ala-Ser(Bzl)Chg-Gln-Ser(Bzl)Leu-PAM Resin (11-1).

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Acetic acid was used for the introduction of the N terminal acetyl group. Removal of the Boc group was performed using 50% TFA in methylene chloride and the TFA salt neutralized with diisopropylethylamine. At the completion of the synthesis the peptide resin was dried to yield Intermediate 11-1.

Step B: [N-Ac-(4-trans-L-Hyp)]-Ala-Ser-Chg-Gln-Ser-Leu-OH (11-2)

The protected peptide resin (11-1), 1.2 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield Intermediate 11-2.

Step C: [N-Ac-(4-trans-L-Hyp)]-Ala-Ser-Chg-Gln-Ser-Leu-Dox The above described intermediate (11-2), 1.157 g (1.45 mmol) was dissolved in DMSO (30 ml) and diluted with DMF (30 ml). To the solution was added doxorubicin hydrochloride, 516 mg (0.89 mmol) followed by 0.310 ml of diisopropylethylamine (1.78 mmol). The stirred solution was cooled (0° C.) and 0.276 ml of diphenylphosphoryl azide (1.28 mmol) added. After 30 minutes, an additional 0.276 ml (1.28 mmol) of DPPA was added and the pH adjusted to ˜7.5 (pH paper) with diisopropylethylamine (DIEA). The pH of the cooled reaction (0° C.) was maintained at ˜7.5 with DIEA for the next 3 hours and the reaction stirred at 0-4° C. overnight. After 18 hours, the reaction (found to be complete by analytical HPLC, system A) was concentrated to an oil. Purification of the crude product was achieved by preparative HPLC, Buffer A=0.1% NH₄OAc—H₂O; B=CH₃CN. The crude product was dissolved in 400 ml of 100% A buffer, filtered and purified on a C-18 reverse phase HPLC radial compression column (Waters, Delta-Pak, 15 μM, 100 Å). A step gradient of 100% A to 60% A was used at a flow rate of 75 ml/min (UV=214 nm). Homogeneous product fractions (evaluated by HPLC, system A) were pooled and freeze-dried. The product was dissolved in H₂O (300 ml), filtered and freeze-dried to provide the purified title compound.

Physical Properties

The physical/chemical properties of the product of Step C are shown below:

| | |
|---|---|
| Molecular Formula: | C₆₂H₈₅N₉O₂₃ |
| Molecular Weight: | 1323.6 |
| High Resolution ES Mass Spec: | 1341.7 (NH₄⁺) |
| HPLC: | System A |
| Column: | Vydac 15 cm #218TP5415, C18 |
| Eluant: | Gradient 95:5 (A:B) to 5:95 (A:B) over |
| | 45 min. A = 0.1% TFA/H₂O, B = 0.1% |
| | TFA/Acetonitrile |
| Flow: | 1.5 ml/min. |
| Wavelength: | 214 nm, 254 nm |
| Retention Time: | 18.2 min. |

Amino Acid Compositional Analysis¹:

| Theory | Found |
|---|---|
| Ala (1) | 1.00 |
| Ser (2) | 1.88 |
| Chg (1) | 0.91 |
| Gln² (1) | 1.00 (as Glu) |
| Hyp (1) | 0.80 |
| Leu (1) | 1.01 |
| Peptide Content: | 0.657 μmol/mg |

Note: ¹20 hr., 100° C., 6N HCl
²Gln converted to Glu

### EXAMPLE 12

Preparation of [N-Glutaryl-(4-trans-L-Hyp)]-Ala-Ser-Chg-Gln-Ser-Leu-Dox (Compound B)

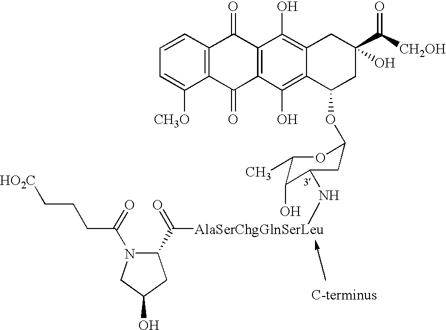

Step A: [N-Glutaryl(OFm)-(4-trans-L-Hyp)]-Ala-Ser-Chg-Gln-Ser-Leu-PAM Resin Starting with 0.5 mmol (0.67 g) Boc-Leu-PAM resin, the protected peptide was synthesized on a 430 Å ABI peptide synthesizer. The protocol used a 4 fold excess (2 mmol) of each of the following protected amino acids: Fmoc-Ser(tBu), Fmoc-Gln(Trt), Fmoc-Chg, Fmoc-Ala, Boc-(4-trans-LHyp). Coupling was achieved using DCC and HOBT activation in methyl-2-pyrrolidinone. The intermediate mono fluorenylmethyl ester of glutaric acid [Glutaryl(OFm)] was used for the introduction of the N-terminal glutaryl group. Removal of the Fmoc group was performed using 20% piperidine. The acid sensitive protecting groups, Boc, Trt and tBu, were removed with 50% TFA in methylene chloride. Neutralization of the TFA salt was with diisopropylethylamine. At the completion of the synthesis, the peptide resin was dried to yield the title compound.

Step B: [N-Glutaryl(OFm)-(4-trans-L-Hyp)]-Ala-Ser-Chg-Gln-Ser-Leu-OH

The protected peptide resin from Step A, 1.2 g, was treated with HF (20 ml) for 1 hr at 0° C. in the presence of anisole (2 ml). After evaporation of the HF, the residue was washed with ether, filtered and extracted with DMF. The DMF filtrate (75 ml) was concentrated to dryness and triturated with H₂O. The insoluble product was filtered and dried to provide the title compound.

Step C: [N-Glutaryl(OFm)-(4-trans-L-Hyp)]-Ala-Ser-Chg-Gln-Ser-Leu-Dox

The above prepared intermediate from Step B, (1.33 g, 1.27 mmol) was dissolved in DMSO (6 ml) and DMF (69 ml). To the solution was added doxorubicin hydrochloride, 599 mg (1.03 mmol) followed by 376 μl of diisopropylethylamine (2.16 mmol). The stirred solution was cooled (0° C.) and 324 μl of diphenylphosphoryl azide (1.5 mmol) added. After 30 minutes, an additional 324 μl of DPPA was added and the pH adjusted to ˜7.5 (pH paper) with diisopropylethylamine (DIEA). The pH of the cooled reaction (0° C.) was maintained at ˜7.5 with DIEA for the next 3 hours and the reaction stirred at 0-4° C. overnight. After 18 hours, the reaction (found to be complete by analytical HPLC, system A) was concentrated to provide the title compound as an oil.

Step D: [N-Glutaryl-(4-trans-L-Hyp)]-Ala-Ser-Chg-Gln-Ser-Leu-Dox

The above product from Step C was dissolved in DMF (54 ml), cooled (0° C.) and 14 ml of piperidine added. The solution was concentrated to dryness and purified by preparative HPLC. (A=0.1% NH₄OAc—H₂O; B=CH₃CN.) The crude product was dissolved in 100 ml of 80% A buffer, filtered and purified on a C-18 reverse phase HPLC radial compression column (Waters, Delta-Pak, 15μ, 100 Å). A step gradient of 80% A to 67% A was used at a flow rate of 75 ml/min (uv=214 nm). Homogeneous product fractions (evaluated by HPLC, system A) were pooled and freeze-dried. The product was further purified using the above HPLC column. Buffer A=15% acetic acid-H₂O; B=15% acetic acid-methanol. The product was dissolved in 100 ml of 20% B/80% A buffer and purified. A step gradient of 20% B to 80% B was used at a flow rate of 75 ml/min (uv=260 nm). Homogeneous product fractions (evaluated by HPLC, system A) were pooled, concentrated and freeze-dried from H₂O to yield the purified title compound.

| | |
|---|---|
| High Resolution | 1418.78 (Na⁺) |
| ES Mass Spec: | |
| HPLC: | System A |
| Column: | Vydac 15 cm #218TP5415, C18 |
| Eluant: | Gradient 95:5 (A:B) to 5:95 (A:B) over 45 min. |
| | A = 0.1% TFA/H₂O, B = 0.1% TFA/Acetonitrile |
| Flow: | 1.5 ml/min. |
| Wavelength: | 214 nm, 254 nm |
| Retention Time: | 18.3 min. |

Amino Acid Compositional Analysis¹:

| Theory | Found |
|---|---|
| Ala (1) | 0.99 |
| Ser (2) | 2.02 |
| Chg (1) | 1.00 |
| Gln² (1) | 1.01 (as Glu) |
| Hyp (1) | 0.99 |
| Leu (1) | 1.00 |
| Peptide Content: | 0.682 μmol/mg |

Note: ¹20 hr., 100° C., 6N HCl
²Gln converted to Glu

### EXAMPLE 13

Preparation of (4-trans-L-Hyp)-Ala-Ser-Chg-Gln-Ser-Leu-Dox

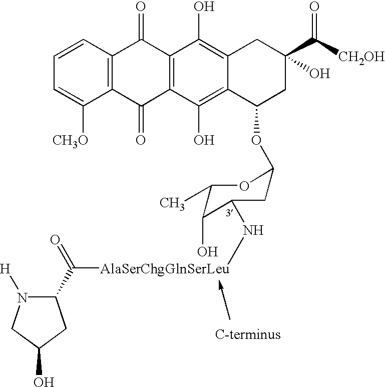

Step A: Fmoc-(4-trans-L-Hyp(Bzl))-Ala-Ser(Bzl)Chg-Gln-Ser(Bzl)Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser (Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Fmoc-OSu (succinamidyl ester of Fmoc) was used for the introduction of the N-terminal protecting group. Removal of the Boc group was performed using 50% TFA in methylene chloride and the TFA salt neutralized with diisopropylethylamine. At the completion of the synthesis the peptide resin was dried to yield the title intermediate.

Step B: Fmoc-(4-trans-L-Hyp)-Ala-Ser-Chg-Gln-Ser-Leu-OH

The protected peptide resin from Step A, 1.1 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield the title intermediate.

Step C: Fmoc-(4-trans-L-Hyp)-Ala-Ser-Chg-Gln-Ser-Leu-Dox

The intermediate from Step B, 0.274 g, was dissolved in DMSO (10 ml) and diluted with DMF (10 ml). To the solution was added doxorubicin hydrochloride, 104 mg followed by 62 μL of diisopropylethylamine (DIEA). The stirred solution was cooled (0° C.) and 56 μL of diphenylphosphoryl azide added. After 30 minutes, an additional 56 μL of DPPA was added and the pH adjusted to 7.5 (pH paper) with DIEA. The pH of the cooled reaction (0° C.) was maintained at ˜7.5 with DIEA. After 4 hours, the reaction (found to be complete by analytical HPLC, system A) was concentrated to an oil. HPLC conditions, system A.

Step D: (4-trans-L-Hyp)-Ala-Ser-Chg-Gln-Ser-Leu-Dox

The above product from Step C was dissolved in DMF (10 ml), cooled (0° C.) and 4 ml of piperidine added. The solution was concentrated to dryness and purified by preparative HPLC. (A=0.1% NH₄OAc—H₂O; B=CH₃CN.) The crude product was dissolved in 100 ml of 90% A buffer, filtered and purified on a C-18 reverse phase HPLC radial compression column (Waters, Delta-Pak, 15 μl, 100 Å). A step gradient of 90% A to 65% A was used at a flow rate of 75 ml/min (uv=214 nm). Homogeneous product fractions (evaluated by HPLC, system A) were pooled and freeze-dried.

| | |
|---|---|
| Molecular Formula: | C₆₀H₈₃N₉O₂₂ |
| Molecular Weight: | 1281.56 |
| High Resolution | 1282.59 (MH⁺) |
| ES Mass Spec: | |
| HPLC: | System A |
| Column: | Vydac 15 cm #218TP5415, C18 |
| Eluant: | Gradient 95:5 (A:B) to 5:95 (A:B) over 45 min. |
| | A = 0.1% TFA/H₂O, B = 0.1% TFA/Acetonitrile |
| Flow: | 1.5 ml/min. |
| Wavelength: | 214 nm, 254 nm |
| Retention Time: | 17.6 min. |

Amino Acid Compositional Analysis¹:

| Theory | Found |
|---|---|
| Ala (1) | 1.00 |
| Ser (2) | 1.94 |
| Chg (1) | 0.94 |
| Gln² (1) | 1.05 (as Glu) |
| Hyp (1) | 0.96 |
| Leu (1) | 1.03 |
| Peptide Content: | 0.690 μmol/mg |

Note: ¹20 hr., 100° C., 6N HCl
²Gln converted to Glu

### EXAMPLE 14

des-Acetylvinblastine-4-O-(N-Acetyl-4-trans-L-Hyp-Ser-Ser-Chg-Gln-Ser-Ser-Pro) Ester

Step A: Preparation of 4-des-Acetylvinblastine

A sample of 2.40 g (2.63 mmol) of vinblastine sulfate (Sigma V-1377) was dissolved under N₂ in 135 ml of absolute methanol and treated with 45 ml of anhydrous hydrazine, and the solution was stirred at 20-25° C. for 18 hours. The reaction was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2×) and saturated NaCl (1×). The combined organic layers were dried over anhydrous Na₂SO₄, and the solvent was removed at reduced pressure to yield the title compound as an off-white crystalline solid. This material was stored at −20° C. until use.

Step B: Preparation of 4-des-Acetylvinblastine 4-O-(Prolyl) Ester

A sample of 804 mg (1.047 mmol) of 4-des-acetylvinblastine, dissolved in 3 ml of CH₂Cl₂ and 18 ml of anhydrous pyridine under nitrogen, was treated with 1.39 g of Fmoc-proline acid chloride (Fmoc-Pro-Cl, Advanced Chemtech), and the mixture was stirred for 20 hr at 25° C. When analysis by HPLC revealed the presence of unreacted starting des-acetylvinblastine, another 0.50 g of Fmoc-Pro-Cl was added, with stirring another 20 hours to complete the reaction. Water (ca. 3 ml) was added to react with the excess acid chloride, and the solution was then evaporated to dryness and partitioned between 300 ml of EtOAc and 150 ml of saturated NaHCO₃, followed by washing twice with saturated NaCl. After drying (Na₂SO₄), the solvent was removed under reduced pressure to give an orange-brown residue, to which was added 30 ml of DMF and 14 ml of piperidine, and after 5 minutes the solution was evaporated under reduced pressure to give a orange-yellow semi-solid residue. After drying in vacuo for about 1 hour, approx. 200 ml of H₂O and 100 ml of ether was added to this material, followed by glacial HOAc dropwise with shaking and sonication until complete dissolution had occurred and the aqueous layer had attained a stable pH of 4.5-5.0 (moistened pH range 4-6 paper). The aqueous layer was then washed with 1 100-ml portion of ether, and each ether layer was washed in turn with 50 ml of H₂O. The combined aqueous layers were subjected to preparative HPLC in 2 portions on a Waters C4 Delta-Pak column 15 μM 300A (A=0.1% TFA/H₂O; B=0.1% TFA/CH₃CN), gradient elution 95→70% A/70 min. Pooled fractions yielded, upon concentration and lyophilization, the title compound.

Step C: N-Acetyl-4-trans-L-Hyp-Ser-Ser-Chg-Gln-Ser-Ser-WANG Resin

Starting with 0.5 mmole (0.61 g) of Fmoc-Ser(t-Bu)—WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser (t-Bu)-OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step D: N-Acetyl-4-trans-L-Hyp-Ser-Ser-Chg-Gln-Ser-Ser-OH

One 0.5-mmol run of the above peptide-resin was suspended in 25 ml of TFA, followed by addition of 0.625 ml each of H₂O and triisopropylsilane, then stirring at 25° for 2.0 hours. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

| Column: | Vydac 15 cm #218TP5415, C18 |
|---|---|
| Eluant: | Gradient (95% A → 50% A) over 45 min. |
| | A = 0.1% TFA/H₂O, B = 0.1% |
| | TFA/acetonitrile |
| Flow.: | 1.5 ml/min. |
| High Resolution ES/FT-MS: | 789.3 |

Step E: des-Acetylvinblastine-4-O-(N-Acetyl-4-trans-L-Hyp-Ser-Ser-Chg-Gln-Ser-Ser-Pro) Ester

Samples of 522 mg (0.66 mmol) of the peptide prepared as described in step D and 555 mg (ca. 0.6 mmol) of 4-des-Acetylvinblastine 4-O-(Prolyl) ester from Step B, prepared as above, were dissolved in 17 ml of DMF under N₂. Then 163 mg (1.13 mmol) of 1-hydroxy-7-azabenzotriazole (HOAt) was added, and the pH was adjusted to 6.5-7 (moistened 5-10 range pH paper) with 2,4,6-collidine, followed by cooling to 0° C. and addition of 155 mg (0.81 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 12 hours the reaction was worked up by addition of ˜4 ml of H₂O and, after stirring 1 hour, concentrated to a small volume in vacuo and dissolution in ca. 150 ml of 5% HOAc. and preparative HPLC in two portions on a Waters C₁₈ Delta-Pak column 15 μM 300A (A=0.1% TFA/H2O; B=0.1% TFA/CH₃CN, gradient elution 95→65% A/70 min). Homogeneous fractions containing the later-eluting product (evaluated by HPLC, system A, 95→65% A/30 minutes) from both runs were pooled and concentrated to a volume of ˜50 ml and passed through approx. 40 ml of AG4X4 ion exchange resin (acetate cycle), followed by freeze-drying to give the title compound as a lyophilized powder.

High Resolution ES/FT-MS: 1637.0

### EXAMPLE 15

des-Acetylvinblastine-4-O—(N-Acetyl-4-trans-L-Hyp-Ser-Ser-Chg-Gln-Ser-Ser-Pro) Ester Acetate

A sample of 4.50 g (3.7 mmol) of 4-O-(prolyl) des-acetylvinblastine TFA salt, prepared as described in Example 14, Step B, was dissolved in 300 ml of DMF under N₂, and the solution was cooled to 0° C. Then 1.72 g (10.5 mmol) of 3,4-dihydro-3-hydroxy-4-oxo-1,2,3-benzotriazine (ODHBT) was added, and the pH was adjusted to 7.0 (moistened 5-10 range pH paper) with N-methylmorpholine (NMM), followed by the addition of 4.95 g (5.23 mmol) of the N-acetyl-heptapeptide of Example 14, Step D, portionwise allowing complete dissolution between each addition. The pH was again adjusted to 7.0 with NMM, and 1.88 g (9.8 mmol) of 1-(3-dimethylaminopropyl)-3-ethylcarbodiimide hydrochloride (EDC) was added, followed by stirring of the solution at 0-5° C. until completion of the coupling as monitored by analytical HPLC (system A), maintaining the pH at ca. 7 by periodic addition of NMM. The analysis showed the major component at 26.3 minutes retention time preceded by aminor component (ca. 10%) at 26.1 minutes, identified as the D-Ser isomer of the title compound. After 20 hours the reaction was worked up by addition of 30 ml of H₂O and, after stirring 1 hour, concentrated to a small volume in vacuo and dissolution in ca. 500 ml of 20% HOAc. and preparative HPLC in 12 portions on a Waters C18 Delta-Pak column 15 mM 300A (A=0.1% TFA/H₂O; B=0.1% TFA/CH₃CN), gradient elution 85→65% A/90 min) at a flow rate of 80 ml/min.

Homogeneous fractions (evaluated by HPLC, system C) representing approx. one-fourth of the total run were pooled and concentrated to a volume of ˜150 ml and passed through approx. 200 ml of Bio-Rad AG4X4 ion exchange resin (acetate cycle), followed by freeze-drying of the eluant gave the acetate salt of the title compound as a lyophilized powder: retention time (system A) 26.7 minutes, 98.9% pure; high resolution ES/FT-MS m/e 1636.82; amino acid compositional analysis 20 hours, 100° C., 6N HCl (theory/found), Ser4/3.91 (corrected), Glu 1/0.92 (Gln converted to Glu), Chg 1/1.11, Hyp 1/1.07, Pro 1/0.99, peptide content 0.516 mmol/mg.

Further combination of homogeneous fractions and purification from side fractions, processing as above through approx. 500 ml of ion exchange resin, afforded an additional amounts of the title compound.

HPLC conditions, system A:

| Column: | Vydac 15 cm #218TP5415, C18 |
|---|---|
| Flow: | 1.5 ml/min. |
| Eluant: | Gradient (95% A → 50% A) over 45 min. |
| A = 0.1% TFA/H₂O, B = 0.1% TFA/acetonitrile | |
| Wavelength: | 214 nm, 280 nm |

HPLC conditions, system C:

| Column: | Vydac 15 cm #218TP5415, C18 |
|---|---|
| Flow: | 1.5 ml/min. |
| Eluant: | Gradient (85% A → 65% A) over 30 min. |
| A = 0.1% TFA/H₂O, B = 0.1% TFA/acetonitrile | |
| Wavelength: | 214 nm, 280 nm |

### EXAMPLE 16

Preparation of 4-des-Acetylvinblastine-23-(4′-aminomethylbicyclo-[2.2.2] octane)methylamide (BDAM-(dAc)vinblastine)

Step A: Preparation of 4-des-Acetylvinblastine-23-hydrazide

A sample of 3.99 g (4.38 mmol) of vinblastine sulfate (Sigma V-1377) was dissolved in 30.4 ml of 1:1 (v/v) absolute ethanol/anhydrous hydrazine, under N₂, and the solution was heated in an oil bath at 60-65° C. for 23 hours. Upon cooling, the solution was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2×) and saturated NaCl (1×). The combined organic layers were dried over anhydrous Na₂SO₄, and the solvent was removed in vacuo to yield, after drying 20 hours in vacuo, the title compound as a white crystalline solid. This material was dissolved in 82 ml of dry, degassed DMF for storage at −20° C. until use (conc. 36 mg/ml).

Step B: Boc-4-aminomethylbicyclo-[2.2.2]octane carboxylic Acid

A sample of 8.79 g (40.0 mmol) of 4-carboxybicyclo-[2.2.2]octanemethylamine hydrochloride salt suspended in 100 ml each of THF and H₂O was treated with 20.0 ml (14.6 g=3.3 equiv.) of TEA, followed by 11.8 g (47.9 mmol) of BOC-ON reagent. All went into solution, and after stirring 24 hours the solution was concentrated in vacuo to a volume of about 50 ml and partitioned between 100 ml of ether and 300 ml of H₂O. After addition of about 2 ml of TEA the aqueous layer was washed with ether (3×), each ether in turn washed with H₂O, and the combined aqueous layer was acidified with 5% KHSO₄ to give the title compound as a white solid, isolated by filtration and drying in vacuo.

Step C: Boc-4-aminomethylbicyclo-[2.2.2]octane Carboxamide

A stirred solution under N₂ of 12.0 g (42.5 mmol) of the product from step B in 100 ml of DMF was treated with 8.0 g (49.3 mmol) of carbonyldiimidazole. After 30 minutes, the DMF was evaporated in vacuo to afford 50-60 ml of a light brown paste, which was stirred and treated with 70 ml of conc. NH₄OH rapidly added. The initial solution turned to a white paste within 30 minutes, after which H₂O was added up to a total volume of 400 ml to complete precipitation of product, which was triturated and isolated by filtration and washing with H₂O, and dried in vacuo to yield the title compound as a white solid.

Step D: Boc-4-aminomethylbicyclo-[2.2.2]octane Nitrile

A solution of 7.52 g (26.6 mmol) of the product from step C in 50 ml of CH₂Cl₂ and 80 ml of anhydrous pyridine was treated with 11.12 g of (methoxycarbonylsulfamoyl)-triethyl-ammonium hydroxide inner salt (Burgess reagent) in 1-g portions over 5 minutes. After stirring for 1.5 hours, TLC (90-10-1, CHCl₃—CH₃OH—H₂O) showed complete conversion to product, and the solution was evaporated to give a paste, to which H₂O was added, up to 400 ml, with trituration and stirring to afford, after standing 20 hr at 0° C., filtration and drying in vacuo, the title compound as a white solid.

Step E: Boc-4-aminomethylbicyclo-[2.2.2]octane Methylamine

A solution of 6.75 g (25.5 mmol) of the product from step D in 200 ml of CH₃OH plus 4 ml of HOAc and 2 ml of H₂O was hydrogenated over 1.63 g of PtO₂ in a Parr shaker at 55 psi for 22 hours. The catalyst was removed by filtration through Celite, and the filtrate was concentrated in vacuo to an oily residue, which was flushed/evaporated with CH₃OH (1×) and CH₂Cl₂ (2×). Product began to crystallize toward the end of the evaporation, and ether (up to 300 ml) was added to complete the precipitation. The white solid was triturated and isolated by filtration and washing with ether to give, after drying in vacuo, the title compound as the acetate salt.

400 Mhz ¹H-NMR (CDCl₃): δ (ppm, TMS) 4.5 (1s, Boc-NH); 2.9 (2br d, —CH₂-NH-Boc); 2.45 (2br s, —CH₂—NH₂); 2.03 (3s, CH₃COOH);1.45 (9s, Boc); 1.40 (12s, ring CH₂).

Step F: Preparation of 4-des-Acetylvinblastine-23-(4′-aminomethylbicyclo-[2.2.2]octane) Methylamide (BDAM-(dAc)vinblastine)

A 30-ml aliquot of the above DMF solution of 4-desacetylvinblastine-23-hydrazide (1.41 mmol), cooled to −15° C. under Argon, was converted to the azide in situ by acidification with 4M HCl in dioxane to pH<1.5 (moistened 0-2.5 range paper), followed by addition of 0.27 ml (1.3 equiv) of isoamyl nitrite and stirring for 1 hour at 10-15° C. The pH was brought to 7 by the addition of DIEA, and a slurry of 1.27 g (3.8 mmol) of the Boc diamine product from step E above in 20 ml of DMF was then added, and the reaction was allowed to warm slowly to 15-20° C. over 2 hours, at which point coupling was complete, as monitored by analytical HPLC (A=0.1% TFA/H₂O; B=0.1% TFA/CH₃CN). The solvent was removed in vacuo and the residue partitioned between EtOAc and 5% NaHCO₃, the organic layer washed with 5% NaCl, and the aqueous layers back-extracted with CH₂Cl₂ to assure removal of the intermediary Boc-BDAM-(dAc)vinblastine. The combined organic layers were dried over Na₂SO₄, the solvent was removed under reduced pressure, and the residue, after flush/evaporation twice from CH₂Cl₂, was dissolved in 30 ml of CH₂Cl₂ and treated with 30 ml of TFA for 30 minutes. The solvents were rapidly removed in vacuo, and the residue was dissolved in 300 ml of 10% HOAc for purification by preparative HPLC in 5 portions on a Waters C4 Delta-Pak column 15 μM 300A (A=0.1% TFA/H₂O; B=0.1% TFA/CH₃CN), gradient elution 95→70% A/60 min, isocratic 70%/20 min. Homogeneous fractions (evaluated by HPLC, system A, 95→50% A) from the five runs were pooled and concentrated in acuo, followed by freeze-drying to give of the title compound as the lyophilized TFA salt.

HPLC conditions, system A:

| Column: | Vydac 15 cm #218TP5415, C18 |
|---|---|
| Eluant: | Gradient (A → B) over 45 min. |
| A = 0.1% TFA/H₂O, B = 0.1% TFA/acetonitrile | |
| Flow: | 1.5 ml/min. |

Retention time: BDAM (dAc) vinblastine 23.5 min. (95% → 50% A)

97% purity

High Resolution ES/FT-MS: 905.63
Compound content by elemental analysis = 0.714 μmol/mg:

N (calc) = 9.28 N (found) = 6.00

### EXAMPLE 17

Preparation of 4-des-Acetylvinblastine-23-(N-Acetyl-Ser-Ser-Ser-Chg-Gln-Ser-Val-BDAM) Amide Acetate Salt

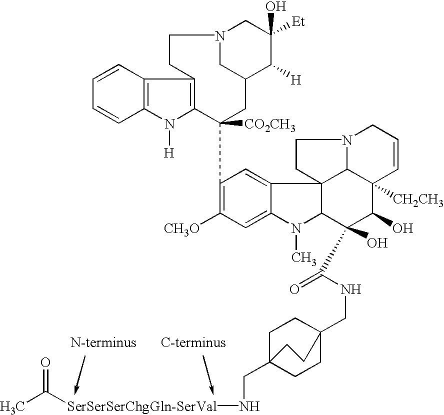

Step A: N-Acetyl-Ser-Ser-Ser-Chg-Gln-Ser-Val-PAM Resin

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH; and acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-Acetyl-Ser-Ser-Ser-Chg-Gln-Ser-Val-OH

Three 0.5-mmol runs of the above peptide-resin (3.5 g) were combined and treated with liquid HF (65 ml) for 1.5 hr at 0° C. in the presence of anisole (6 ml). After evaporation of the HF, the residue was washed with ether, filtered and leached with 150 ml of DMF in several portions, adding DIEA to pH ˜8, followed by removal of the DMF in vacuo to a volume of 100 ml. The concentration was determined as ca. 11.7 mg/ml (by weighing the dried resin before and after leaching. The sample purity was determined as 96% by HPLC. The solution was used directly for conjugation with BDAM-(dAc) vinblastine.

Step C: 4-Des-acetylvinblastine-23-(N-Acetyl-Ser-Ser-Ser-Chg-Gln-Ser-Val-BDAM) Amide Acetate Salt

To 58 ml (equivalent to 0.875 mmol of peptide) of the solution from step B was added 530 mg (0.520 mmol) of BDAM-(dAc)vinblastine, prepared as described in Example 16, Step F, under N₂, cooling to 0° C., and the pH was adjusted to ˜8 (moistened 5-10 range pH paper) with DIEA. Then 0.134 ml (0.62 mmol) of DPPA was added, followed by stirring at 0-5° C. until completion of the coupling as monitored by analytical HPLC (A=0.1% TFA/H₂O; B=0.1% TFA/CH₃CN), maintaining the pH at ≧7 by periodic addition of DIEA. After 24 hours, the reaction was worked up by addition of 10 ml of H₂O, stirring 1 hour and concentration to small volume in vacuo, then dissolution in ca. 100 ml of 10% HOAc/5% CH₃CN, adjustment of the pH to 5 with NH₄HCO₃, filtration to remove insolubles, and preparative HPLC in 3 portions on a Waters C4 Delta-Pak column 15 μM 300A (A=0.1% NH₄HCO₃/H₂O; B=CH₃CN), gradient elution 95→40% A/70 min. Fractions from each run containing product were pooled, acidified to pH 3 with glacial HOAc, concentrated in vacuo to a volume of ˜50 ml, and purified by preparative HPLC on a Waters C18 Delta-Pak column 15 μM 300A (A=0.1% TFA/H₂O; B=0.1% TFA/CH₃CN), gradient elution 95→70% A/60 min, isocratic 70%/20 min. Homogeneous fractions (evaluated by HPLC, system A, 95→50% A) from all three runs were pooled and concentrated to a volume of ˜100 ml., diluted with 5% CH₃CN, and passed through AG4X4 ion exchange resin (acetate cycle), followed by freeze-drying to give the title compound as a lyophilized powder.

HPLC conditions, system A:

| Column: | Vydac 15 cm #218TP5415, C18 |
|---|---|
| Eluant: | Gradient (A → B) over 45 min. |
| A = 0.1% TFA/H₂O, B = 0.1% TFA/acetonitrile | |
| Flow: | 1.5 ml/min. |

Retention times:

| | |
|---|---|
| BDAM (dAc) vinblastine | 23.5 min. |
| N-Acetyl-Ser-Ser-Ser-Chg-Gln-Ser-Val-OH | 14.5 min. |
| 4-Des-acetylvinblastine-23-( N-Acetyl-Ser-Ser- | 29.5 min. |
| Ser-Chg-Gln-Ser-Val-BDAM) amide | |

High Resolution ES/FT-MS: 1662.03
Amino Acid Compositional Analysis¹ (theory/found):

²Ser4/3.6 ³Glu 1/2.10 ⁴Val 1/0.7 Chg 1/0.95
Peptide content 0.504 μmol/mg
Note: ¹20 hr, 100° C., 6N HCl
²Uncorrected
³Gln converted to Glu
⁴Incomplete hydrolysis

### EXAMPLE 18

Preparation of 4-des-Acetylvinblastine-23-(N-methoxy-diethylene-oxyacetyl-4-trans-L-Hyp-Ser-Ser-Chg-Gln-Ser-Val-BDAM) Amide Acetate Salt

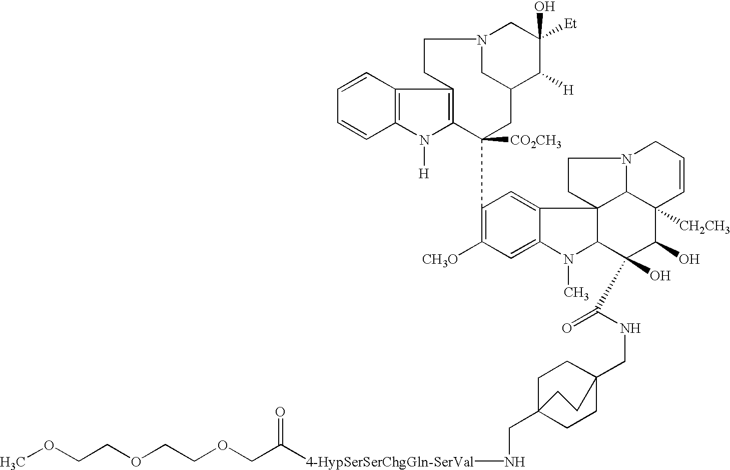

Step A: N-methoxydiethyleneoxyacetyl-4-trans-L-Hyp-Ser-Ser-Chg-Gln-Ser-Val-PAM Resin

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH, Boc-4-trans-Hyp(Bzl)-OH; and 2-[2-(2-methoxyethoxy)-ethoxy]acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-methoxydiethyleneoxyacetyl-4-trans-L-Hyp-Ser-Ser-Chg-Gln-Ser-Val-OH

Two 0.5-mmol runs of the above peptide-resin (2.4 g) were combined and treated with liquid HF (40 ml) for 1.5 hr at 0° C. in the presence of anisole (4 ml). After evaporation of the HF, the residue was washed with ether, filtered and leached with 150 ml of H₂O in several portions, followed by preparative HPLC on a Waters C18 Delta-Pak column 15 μM 100A (A=0.1% TFA/H₂O; B=0.1% TFA/CH₃CN), gradient elution 95→70% A/70 min, and pooling of homogeneous fractions and freeze drying to give the title compound as lyophilized powder. The sample purity was determined as 99% by HPLC.

Step C: 4-des-Acetylvinblastine-23-(N-methoxydiethylene-oxyacetyl-4-trans-L-Hyp-Ser-Ser-Chg-Gln-Ser-Val-BDAM) Amide Acetate Salt

Samples of 440 mg (0.47 mmol) of the peptide from step B and 340 mg (0.33 mmol) of BDAM-(dAc)vinblastine, prepared as described in Example 15, Step F, were dissolved in 25 ml of DMF under N₂, cooling to 0° C. Then 85 mg (0.63 mmol) of 1-hydroxy-7-azabenzotriazole (HOAt) was added, and the pH was adjusted to 6.5-7 (moistened 5-10 range pH paper) with 2,4,6-collidine, followed by addition of 117 mg (0.61 mmol) of 1-(3-dimethylaminopropyl)-3-ethylcarbodiimide hydro-chloride (EDC). Stirring was continued at 0-5° C. until completion of the coupling as monitored by analytical HPLC (A=0.1% TFA/H2O; B=0.1% TFA/CH₃CN), maintaining the pH at 6.5-7 by periodic addition of 2,4,6-collidine. After 3 hours the reaction was worked up by addition of ˜10 ml of H₂O, stirring 1 hour and concentration to small volume in vacuo, then dissolution in ca. 70 ml of 5% HOAc. and preparative HPLC on a Waters C18 Delta-Pak column 15 μM 300A (A=0.1% TFA/H₂O; B=0.1% TFA/CH₃CN), gradient elution 95→40% A/70 min). Homogeneous fractions (evaluated by HPLC, system A, 95→50% A) from all three runs were pooled and concentrated to a volume of ˜50 ml and passed through AG4X4 ion exchange resin (acetate cycle), followed by freeze-drying to give the title compound as a lyophilized powder.

HPLC conditions, system A:

Column: Vydac 15 cm #218TP5415, C18
Eluant: Gradient (A → B) over 45 min.
A = 0.1% TFA/H₂O, B = 0.1% TFA/acetonitrile
Flow: 1.5 ml/min.

Retention times:

| | |
|---|---|
| BDAM (dAc) vinblastine | 23.5 min. |
| N-methoxydiethyleneoxyacetyl-4-trans- | 16.2 min. |

L-Hyp-Ser-Ser-Chg-Gln-Ser-Val-OH

| | |
|---|---|
| 4-des-Acetylvinblastine-23- | 29.6 min. |
| (N-methoxydiethyleneoxyacetyl- | |

4-trans-L-Hyp-Ser-Ser-Chg-Gln-Ser-Val-BDAM) amide

High Resolution ES/FT-MS: 1805.95
Amino Acid Compositional Analysis¹ (theory/found):
²Ser3/1.7 ³Glu 1/1.01 ⁴Val 1/0.93 Chg 1/0.98 Hyp 1/1.01

Peptide content = 0.497 μmol/mg
Note: ¹20 hr, 100° C., 6N HCl
²Uncorrected
³Gln converted to Glu
⁴Incomplete hydrolysis

### EXAMPLE 19

Preparation of 4-des-Acetylvinblastine-23-(N-Acetyl-4-trans-L-Hyp-Ser-Ser-Chg-Gln-Ser-HCAP) Amide Acetate Salt (19-7)

Step A: N-Acetyl-4-trans-L-Hyp-Ser-Ser-Chg-Gln-OH (19-1) Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Chg-OH, Fmoc-4-trans-Hyp(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA:2.5% H20:2.5% Triisopropylsilane (20 ml) for 2 hours at room temperature under argon. After evaporation of the TFA, the residue was washed with ether, filtered and dried to give crude peptide which was purified by preparatory HPLC on a Delta-Pak C18 column with 0.1% trifluoroacetic acid aqueous acetonitrile solvent systems using 100-70% A, 60 min linear gradient. Fractions containing product of at least 99% (HPLC) purity were combined to give the title compound.

FABMS: 615.3

Peptide Content: 1.03 mmole/mg.

HPLC: 99% pure @214 nm, retention time=10.16 min, (Vydac C₁₈, gradient of 95% A/B to 50% A/B over 30 min, A=0.1% TFA-H₂O, B=0.1% TFA-CH₃CN)

Step B: N-Boc-(1S,2R)-(+)-Norephedrine (19-2)

A solution of 1.51 g (10 mmol) of (1S,2R)-(+)-Norephedrine in a mixture of 1,4 dioxane (20 ml), water (10 ml) and 1N NaOH (10 ml) was stirred and cooled in an ice-water bath. Di-(t-butyl) dicarbonate (2.4 g, 11 mmol) was added in portions over approx. 20 minutes. The reaction was stirred in the cold for 2 hours, then at room temperature for an additional 1 hour. The solution was concentrated to remove most of the dioxane, cooled in an ice bath and covered with a layer of ethyl acetate (30 ml) and acidified to pH 2 with 1N KHSO4. The aqueous phase was extracted 2×with EtOAc. The combined extracts were washed with water, brine and were concentrated and dried to provide the desired product as a white crystalline solid (19-2). FABMS: 252

Step C: N-Boc-HCAP (19-3)

A solution of 2.38 g of N-Boc-(1S,2R)-(+)-Norephedrine (19-2) in 50 ml acetic acid/10 ml H₂O was hydrogenated at 60 psi on a Parr apparatus over 500 mg of Ir black catalyst for 24 hours. The reaction was filtered through a Celite pad, and the filtrate concentrated in vacuo to give a tan foam (19-3). FABMS: 258.2

Step D: N-Benzyloxycarbonyl-Ser-N-t-Boc-HCAP Ester (19-4)

A solution of 1.95 g (6.6 mmol) of N-Z-Ser(tBu)-OH, 1.54 g (6.0 mmol) of N-Boc-HCAP (19-3), 1.26 g (6.6 mmol) of EDC, and 146 mg (1.2 mmol) of DMAP in 30 ml of anh. CH₂Cl₂ was treated and the resulting solution stirred at room temperature in an N₂ atmosphere for 12 hours. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (19-4).

Step E: H-Ser(tBu)-N-t-Boc-HCAP Ester (19-5)

A 2.0 g of (19-4) in a solution of 90 ml EtOH, 20 ml water, and 10 ml acetic acid was hydrogenated on a Parr apparatus at 50 psi over 200 mg of Pd(OH)₂ catalyst for 3 hours. The reaction was filtered through a Celite pad, and the filtrate was concentrated to small volume in vacuo, then purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the intermediate (19-5). FABMS: 401.3

Step F: N-Acetyl-4-trans-L-Hyp-Ser-Ser-Chg-Gln-Ser-HCAP Amine (19-6)

A solution of 614 mg (1.0 mmol) of N-Acetyl-4-trans-L Hyp-Ser-Ser-Chg-Gln-OH (19-1), 400 mg (1.0 mmol) of H-Ser(tBu)-N-t-Boc-HCAP ester (19-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 hours. The solvent was removed in vacuo, the residue was washed with ether and dried. The crude product was treated with 95% TFA:5% H₂O (20 ml) for 2 hours at room temperature under argon. After evaporation of the TFA, the residue was purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the intermediate compound (19-6).

FABMS: 841.8

Peptide Content: 863.39 NMole/mg.

HPLC: 99% pure @214 nm, retention time=13.7 min, (Vydac C₁₈, gradient of 95% A/B to 5% A/B over 30 min, A=0.1% TFA-H₂O, B=0.1% TFA-CH₃CN)

Step G: 4-des-Acetylvinblastine-23-(N-Ac-4-trans-L-Hyp-Ser-Ser-Chg-Gln-Ser-HCAP) Amide Acetate Salt (19-7)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hour at 10-15° C. The pH was brought to 7 by the addition of DIEA, and 555 mg (0.66 mmol) of amine derivative (19-6) from step F was then added, and the reaction was stirred at 0° C. for 24 hours, and purified by preparatory HPLC on a 15 μM, 100A, Delta-Pak C18 column with 0.1% trifluoroacetic acid-aqueous acetonitrile solvent systems using 95-50% A, 60 min linear gradient. Homogeneous fractions were pooled and concentrated in vacuo, followed by freeze-drying to give the title compound as the TFA salt which was converted to the corresponding HOAc salt by AG4X4 resin (100-200 mesh, free base form, BIO-RAD) (19-7).

ES: 1576.7

Peptide Content: 461.81 NMole/mg.

Ser 3.04; Hyp 1.07; Chg 1.02; Glu 1.00

HPLC: 99% pure @214 nm, retention time=18.31 min, (Vydac C₁₈, gradient of 95% A/B to 5% A/B over 30 min, A=0.1% TFA-H₂O, B=0.1% TFA-CH₃CN)

### EXAMPLE 20

Preparation of 4-des-Acetylvinblastine-23-(N-Acetyl-Ser-Chg-Gln-Ser-Ser-Pro-HCAP) Amide Acetate Salt (20-5)

Step A: N-Acetyl-Ser-Chg-Gln-Ser-Ser-OH (20-1)

Starting with 0.5 mmole (0.80 g) of Fmoc-Ser(tBu)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Gln-OH, Fmoc-Chg-OH, Fmoc-Ser(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA:2.5% H₂O:2.5% Triisopropylsilane (20 ml) for 2 hours at room temperature under argon. After evaporation of the TFA, the residue was washed with ether, filtered and dried to give crude peptide which was purified by preparatory HPLC on a Delta-Pak C18 column with 0.1% trifluoroacetic acid aqueous acetonitrile solvent systems using 100-70% A, 60 min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C₁₈, gradient of 95% A/B to 50% A/B over 30 min, A=0.1% TFA-H₂O, B=0.1% TFA-CH₃CN)

Step B: N-Boc-(1S,2R)-(+)-Norephedrine (19-2)

A solution of 1.51 g (10 mmol) of (1S,2R)-(+)-Norephedrine in a mixture of 1,4 dioxane (20 ml), water (10 ml) and 1N NaOH (10 ml) is stirred and cooled in an ice-water bath. Di-(t-butyl) dicarbonate (2.4 g, 11 mmol) was added in portions over approx. 20 minutes. The reaction was stirred in the cold for 2 hours, then at room temperature for an additional 1 hour. The solution was concentrated to remove most of the dioxane, cooled in an ice bath and covered with a layer of ethyl acetate (30 ml) and acidified to pH 2 with 1N KHSO4. The aqueous phase was extracted 2×with EtOAc. The combined extracts were washed with water, brine and were concentrated and dried to provide the desired product as a white crystalline solid. FABMS: 252

Step C: N-Boc-HCAP (19-3)

A solution of 2.38 g of N-Boc-(1S,2R)-(+)-Norephedrine (19-2) in 50 ml acetic acid/10 ml H₂O was hydrogenated at 60 psi on a Parr apparatus over 500 mg of Ir black catalyst for 24 hours. The reaction was filtered through a Celite pad, and the filtrate concentrated in vacuo to give a tan foam. FABMS: 258.2

Step D: N-Benzyloxycarbonyl-Pro-N-t-Boc-HCAP Ester (20-2)

A solution of 1.62 g (6.6 mmol) of N-Z-Pro-OH, 1.54 g (6.0 mmol) of N-Boc-HCAP (19-3), 1.26 g (6.6 mmol) of EDC, and 146 mg (1.2 mmol) of DMAP in 30 ml of anh. CH₂Cl₂ was treated and the resulting solution stirred at room temperature in an N₂ atmosphere for 12 hours. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E: H-Pro-N-t-Boc-HCAP Ester (20-3)

A 2.0 g of (20-2) in a solution of 90 ml EtOH, 20 ml water, and 10 ml acetic acid was hydrogenated on a Parr apparatus at 50 psi over 200 mg of Pd(OH)₂ catalyst for 3 hours. The reaction was filtered through a Celite pad, and the filtrate was concentrated to small volume in vacuo, then purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the title compound (20-3). FABMS: 356.3

Step F: N-Acetyl-Ser-Chg-Gln-Ser-Ser-Pro-HCAP Amine (20-4)

A solution of 589 mg (1.0 mmol) of N-Acetyl-Ser-Chg-Gln-Ser-Ser-OH (20-1), 356 mg (1.0 mmol) of H-Pro-N-t-Boc-HCAP ester (20-3), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 hours. The solvent was removed in vacuo, the residue was washed with ether and dried. The crude product was treated with 95% TFA:5% H₂O (20 ml) for 2 hours at room temperature under argon. After evaporation of the TFA, the residue was purified by preparatory HPLC on a Delta-Pak C18 column with 0.1% trifluoroacetic acid-aqueous acetonitrile solvent systems using 95-50% A, 60 min linear gradient. Fractions containing product of at least 99% (HPLC) purity were combined to give the title compound (20-4).

FABMS: 825.5

Peptide Content: 893.6 NMole/mg.

HPLC: 99% pure @214 nm, retention time=15.2 min, (Vydac C18, gradient of 95% A/B to 5% A/B over 30 min, A=0.1% TFA-H₂O, B=0.1% TFA-CH₃CN)

Step G: 4-des-Acetylvinblastine-23-(N-Ac-Ser-Chg-Gln-Ser-Ser-Pro-HCAP) Amide Acetate Salt (20-5)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hour at 10-15° C. The pH was brought to 7 by the addition of DIEA, and 545 mg (0.66 mmol) of amine derivative (20-4) from step F was then added, and the reaction was stirred at 0° C. for 24 hours, and purified by preparatory HPLC on a 15 μM, 100A, Delta-Pak C18 column with 0.1% trifluoroacetic acid-aqueous acetonitrile solvent systems using 95-50% A, 60 min linear gradient. Homogeneous fractions were pooled and concentrated in vacuo, followed by freeze-drying to give the title compound as the TFA salt which was converted to title compound by AG4X4 resin (100-200 mesh, free base form, BIO-RAD) (20-5)

ES⁺: 1560.9

Peptide Content: 586.8 NMole/mg.

Ser 3.04; Chg 1.01; Glu 1.00; Pro 0.97

HPLC: 99% pure @214 nm, retention time=13.4 min, (Vydac C₁₈, gradient of 95% A/B to 5% A/B over 30 min, A=0.1% TFA-H₂O, B=0.1% TFA-CH₃CN)

Biological Assays.

The ability of the compounds useful in the methods of the present invention to act as NK-1 receptor antagonists can be demonstrated using the following assays

### EXAMPLE 21

NK-1 Receptor Binding Assay

NK-1 receptor binding assays are performed in intact Chinese hamster ovary (CHO) cells expressing the human NK-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance P (0.1 nM, 2000 Ci/mmol; New England Nuclear) is incubated in the presence or absence of test compounds (dissolved in 5 μl dimethylsulphoxide, DMSO) with 5×10⁴ CHO cells. Ligand binding is performed in 0.25 ml of 50 mM Tris-HCl, pH 7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2 μg/ml pepstatin, 2 μg/ml leupeptin and 2.8 μg/ml furoyl saccharine. The incubation proceeds at room temperature until equilibrium is achieved (>40 minutes) and the receptor-ligand complex is harvested by filtration over GF/C filters pre-soaked in 0.1% polyethylenimine using a Tomtek 96-well harvester. Non-specific binding is determined using excess substance P (1 μM) and represents <10% of total binding.

Particularly preferred NK-1 receptor antagonists of use in the present invention are compounds which are potent NK-1 receptor antagonists, i.e. compounds with an NK-1 receptor affinity (IC₅₀) of less than 10 nM, favourably less than 2 nM and preferably less than 1 nM.

The following examples illustrate pharmaceutical compositions according to the invention.

### EXAMPLE 22

Tablet Formulation Containing 50-300 mg of NK-1 Antagonist

| | Amount mg | | |
|---|---|---|---|
| NK-1 antagonist | 50.0 | 100.0 | 300.0 |
| Microcrystalline cellulose | 80.0 | 80.0 | 80.0 |
| Modified food corn starch | 80.0 | 80.0 | 80.0 |
| Lactose | 189.5 | 139.5 | 439.5 |
| Magnesium Stearate | 0.5 | 0.5 | 0.5 |

The active ingredient, cellulose, lactose and a portion of the corn starch are mixed and granulated with 10% corn starch paste. The resulting granulation is sieved, dried and blended with the remainder of the corn starch and the magnesium stearate. The resulting granulation is then compressed into tablets containing 50 mg, 100 mg and 300 mg of the NK-1 receptor antagonist per tablet.

### EXAMPLE 23

Parenteral Injection Formulation

| | Amount | |
|---|---|---|
| Active Ingredient | 10 to 300 | mg |
| Citric Acid Monohydrate | 0.75 | mg |
| Sodium Phosphate | 4.5 | mg |
| Sodium Chloride | 9 | mg |
| Water for injection | to 10 | ml |

The sodium phosphate, citric acid monohydrate and sodium chloride are dissolved in a portion of the water. The active ingredient is dissolved or suspended in the solution and made up to volume.

### PSA Conjgate Assays

### EXAMPLE 24

Assessment of the Recognition of Oligopeptide-Cytotoxic Drug Conjugates by Free PSA

The PSA conjugates, prepared as described above and in particular in Examples 11-20, are individually dissolved in PSA digestion buffer (50 mM tris(hydroxymethyl)-aminomethane pH 7.4, 140 mM NaCl) and the solution added to PSA at a molar ration of 100 to 1. Alternatively, the PSA digestion buffer utilized is 50 mM tris(hydroxymethyl)-aminomethane pH 7.4, 140 mM NaCl. The reaction is quenched after various reaction times by the addition of trifluoroacetic acid (TFA) to a final 1% (volume/volume). Alternatively the reaction is quenched with 10 mM ZnCl₂. The quenched reaction is analyzed by HPLC on a reversed-phase C18 column using an aqueous 0.1% TFA/acetonitrile gradient. The amount of time (in minutes) required for 50% cleavage of the noted oligopeptide-cytotoxic agent conjugates with enzymatically active free PSA were then calculated.

### EXAMPLE 25

In vitro Assay of Cytotoxicity of Peptidyl Derivatives of Doxorubicin

The cytotoxicities of a cleaveable oligopeptide-doxorubicin conjugates, prepared as described above and in particular in Examples 11-13, against a line of cells which is known to be killed by unmodified doxorubicin are assessed with an Alamar Blue assay. Specifically, cell cultures of LNCap prostate tumor cells (which express enzymatically active PSA) or DuPRO cells in 96 well plates are diluted with medium (Dulbecco's Minimum Essential Medium-α [MEM-α]) containing various concentrations of a given conjugate (final plate well volume of 200%1). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

### EXAMPLE 26

In vitro Assay of Cytotoxicity of Peptidyl Derivatives of Vinca Drugs

The cytotoxicities of a cleaveable oligopeptide-vinca drug conjugates, prepared as described above and in particular in Examples 14-19, against a line of cells which is known to be killed by unmodified vinca drug was assessed with an Alamar Blue assay. Specifically, cell cultures of LNCap prostate tumor cells, Colo320DM cells (designated C320) or T47D cells in 96 well plates are diluted with medium containing various concentrations of a given conjugate (final plate well volume of 200%1). The Colo320DM cells, which do not express free PSA, are used as a control cell line to determine non-mechanism based toxicity. The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures and an EC₅₀ was determined.

### EXAMPLE 27

In vivo Efficacy of Peptidyl-Cytotoxic Agent Conjugates

LNCaP.FGC or DuPRO-1 cells are trypsinized, resuspended in the growth medium and centifuged for 6 mins. at 200×g. The cells are resuspended in serum-free α-MEM and counted. The appropriate volume of this solution containing the desired number of cells is then transferred to a conical centrifuge tube, centrifuged as before and resuspended in the appropriate volume of a cold 1:1 mixture of α-MEM-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22G needle. Mice are either given approximately 5×10⁵ DuPRO cells or 1.5×10⁷ LNCaP.FGC cells.

Following inoculation with the tumor cells the mice are treated under one of two protocols:

Protocol A:

One day after cell inoculation the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

### EXAMPLE 28

In vitro Determination of Proteolytic Cleavage of Conjugates by Endogenous Non-PSA Proteases

Step A: Preparation of Proteolytic Tissue Extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000×g in a swinging bucket rotor (HB4-5), the pellet is discarded and the re-supernatant centrifuged at 100,000×g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000×g. The supernatant is discarded and the pellet resuspended in Buffer C (10 mM potassium phosphate buffer containing 0.25 M sucrose, pH 7.4), using ½ the volume used above, and homogenized with a dounce homogenizer.

Protein content of the two solutions (cytosol and membrane) is determined using the Bradford assay. Assay aliquots are then removed and frozen in liquid N₂. The aliquots are stored at −70° C.

Step B: Proteolytic Cleavage Assay

For each time point, 20 microgram of PSA conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

### EXAMPLE 29

In vivo Efficacy of Administration of a Combination of a PSA Conjugate and an NK-1 Receptor Antagonist

Male nude mice (4 groups of 15) are injected subcutaneously with 1.5×10⁷ LNCaP.FGC cells (available from the American Type Culture Collection, ATCC No. CR^{L}-1740; see also J. S. Horoszewicz et al. Cancer Res., 43:1809-1818 (1983)) in 80% Matrigel.

Five days after the tumor cell implantation, a test NK-1 receptor antagonist is incorporated into the rodent chow food that is given to the mice ad libitum for the remainder of the test period. The concentration of the NK-1 receptor antagonist in the food is adjusted to provide a therapeutically minimal or subminimal plasma concentration of the NK-1 receptor antagonist. For example, if the compound of Example 10 is being tested in combination with a PSA conjugate, the concentration of the compound in the food is adjusted so that a continuous plasma concentration of between 0.3-1.0 μM is maintained.

Administration of the NK-1 receptor antagonist is as follows:

Group A: Food containing test NK-1 receptor antagonist compound

Group B: Food without NK-1 receptor antagonist

Group C: Food containing test NK-1 receptor antagonist

Group D: Food without NK-1 receptor antagonist

Beginning at the same time as administration of the NK-1 receptor antagonist, a solution of test PSA conjugate is administered to Groups A and B. Vehicle is administered to Groups C and D. The PSA conjugate is administered IV as a therapeutically minimal dose. For example, when the PSA conjugate described in Example 15 is tested, a 0.20 mL of a solution of test PSA conjugate, (3-5 mpk, 34.1 mL D5W+80 μL 7.5% sodium bicarbonate) is administered to Groups A and B. Vehicle (0.20 mL) is administered to Groups C and D.

Three days after the initial dosing of the NK-1 receptor antagonist and the PSA conjugate, three mice from each group are bled from the tail vein to assess serum levels of the test NK-1 receptor antagonist.

After the initial dose of PSA conjugate, the animals are administered PSA conjugate solution either as four additional doses (one/day) of the test PSA conjugate solution or vehicle are administered to the respective Groups over four consecutive days, or once a week for four consecutive weeks

At the end of 5-6 weeks after the inoculation with the LNCaP cells the mice are bled from the tail vein and the plasma PSA level is measured using a Tandem®-E PSA ImmunoEnzyMetri Assay kit (Hybritech). The plasma concentration of the NK-1 receptor antagonist is also determined at this time. The mice are then sacrificed, weighed, tumors excised and weighed.

What is claimed is:

1. A method for treating cancer in a mammal in need thereof which comprises administering to said mammal amounts of at least one tachykinin receptor antagonist and at least one PSA conjugate.

2. The method according to claim 1 wherein an amount of an tachykinin receptor antagonist and an amount of an PSA conjugate are administered consecutively.

3. The method according to claim 1 wherein an amount of an tachykinin receptor antagonist and an amount of an PSA conjugate are administered simultaneously.

4. The method according to claim 1 wherein the cancer is a cancer related to cells that express enzymatically active PSA.

5. The method according to claim 1 wherein the cancer is prostate cancer.

6. The method according to claim 1 wherein the PSA conjugate is selected from:
a) a compound represented by the formula I: 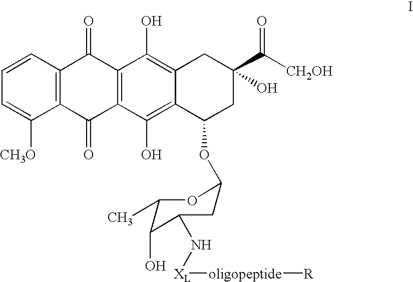
wherein:
oligopeptide is an oligopeptide which is specifically recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine;
R is hydrogen or —(C═O)R¹; and
R¹ is C₁-C₆-alkyl or aryl,
or the pharmaceutically acceptable salt thereof;
b) a compound represented by the formula II: 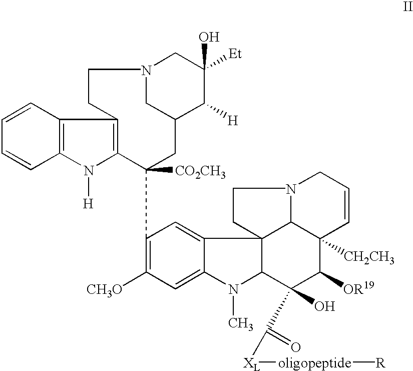
wherein:
oligopeptide is an oligopeptide which is specifically recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine; or
X_{L} is —NH—(CH₂)ₙ—NH—;
R is hydrogen or —(C═O)R¹;
R¹ is C₁-C₆-alkyl or aryl;
R¹⁹ is hydrogen or acetyl; and
n is 1, 2, 3, 4 or 5,
or the pharmaceutically acceptable salt thereof;
c) a compound represented by the formula III: 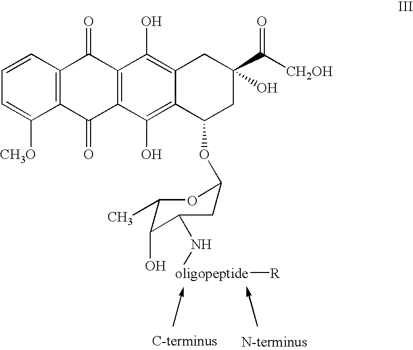
wherein:
oligopeptide is an oligopeptide which is selectively recognized by the free prostate specific antigen (PS A) and is capable of being proteolytically cleaved by the enzymatic activity of the free prostate specific antigen, wherein the oligopeptide comprises a cyclic amino acid of the formula: 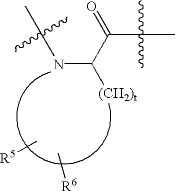
and wherein
the C-terminus carbonyl is covalently bound to the amine of doxorubicin;
R is selected from
a) hydrogen,
b) —(C═O)R^{1a}, 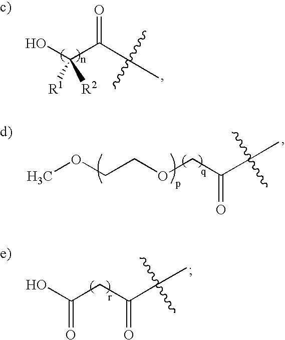
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R⁵ is selected from HO— and C₁-C₆ alkoxy;
R⁶ is selected from hydrogen, halogen, C₁-C₆ alkyl, HO— and C₁-C₆ alkoxy; and
n is 1, 2, 3 or 4;
p is zero or an integer between 1 and 100;
q is 0 or 1, provided that if p is zero, q is 1;
r is an integer between 1 and 10; and
t is 3 or 4;
or a pharmaceutically acceptable salt thereof;
d) a compound represented by the formula IV: 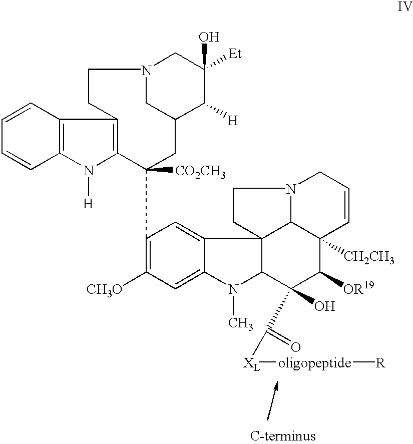
wherein:
oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula: 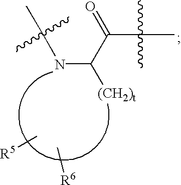
X_{L} is —NH—(CH₂)ᵤ—NH—;
R is selected from
a) hydrogen,
b) —(C═O)R^{1a}, 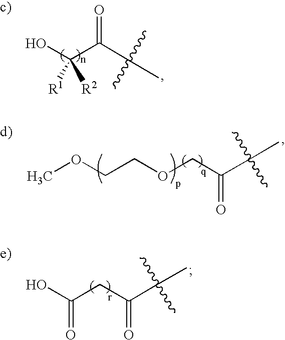
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2 or 3;
t is 3 or 4;
u is 1, 2, 3, 4 or 5;
or the pharmaceutically acceptable salt thereof;
e) a compound represented by the formula V: 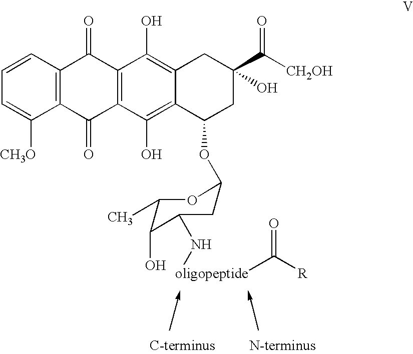
wherein:
oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;
R is selected from 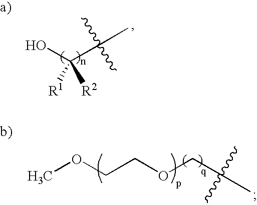
R¹ and R² are independently selected from: hydrogen, OH, C₁-C₆ alkyl, C₁-C₆ alkoxy, C₁-C₆ aralkyl and aryl;
n is 1, 2, 3 or 4;
p is zero or an integer between 1 and 100;
q is 0 or 1, provided that if p is zero, q is 1;
or the pharmaceutically acceptable salt thereof;
f) a compound represented by the formula VI: 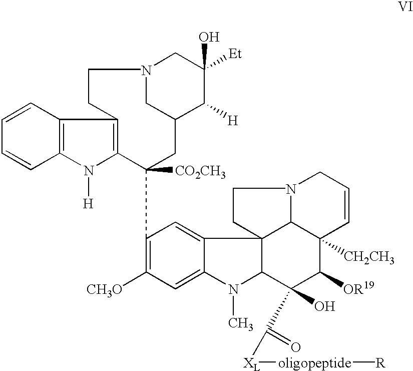
wherein:
oligopeptide is an oligopeptide which is specifically recognized by the free prostate specific antigen (PSA) and is capable of being proteolytically cleaved by the enzymatic activity of the free prostate specific antigen;
X_{L} is —NH—(CH₂)ᵣ—NH—;
R is selected from 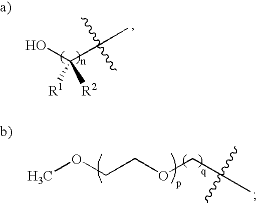
R¹ and R² are independently selected from: hydrogen, OH, C₁-C₆ alkyl, C₁-C₆ alkoxy, C₁-C₆ aralkyl and aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2, 3, 4 or 5;
or the pharmaceutically acceptable salt thereof;
g) a compound represented by the formula VII: 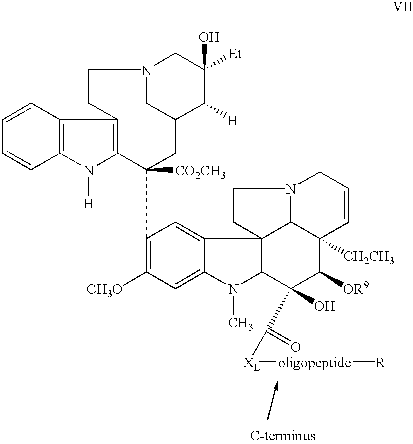
wherein:
oligopeptide is an oligopeptide which is specifically recognized by the free prostate specific antigen (PSA) and is capable of being proteolytically cleaved by the enzymatic activity of the free prostate specific antigen,
X_{L} is —NH—(CH₂)ᵤ—W—(CH₂)ᵤ—NH—;
R is selected from
a) hydrogen,
b) —(C═O)R^{1a}, 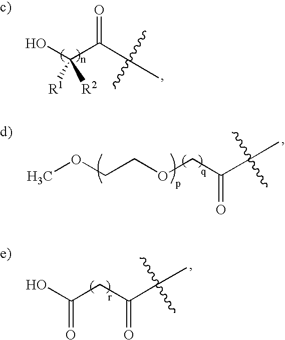
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3;
or the pharmaceutically acceptable salt thereof; and
h) a compound represented by the formula VIII: 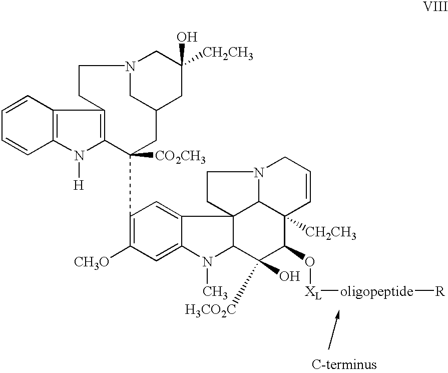
wherein:
oligopeptide is an oligopeptide which is specifically recognized by the free prostate specific antigen (PSA) and is capable of being proteolytically cleaved by the enzymatic activity of the free prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from
a) hydrogen,
b) —(C═O)R^{1a}, 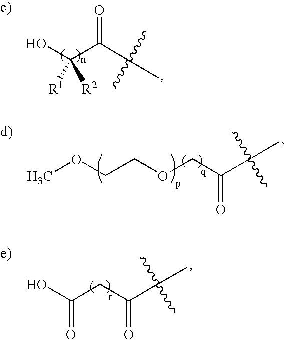
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3;
or the pharmaceutically acceptable salt or optical isomer thereof.

7. The method according to claim 6 wherein the PSA conjugate is selected from:

| | |
|---|---|
| i) | |
| 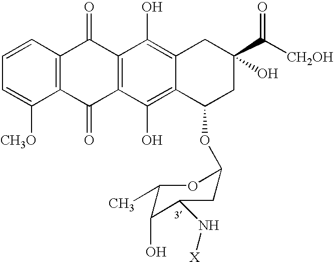 | |
| wherein X is: | |
| AsnLysIleSerTyrGlnSer- | (SEQ.ID.NO.: 1), |
| AsnLysIleSerTyrGlnSerSer- | (SEQ.ID.NO.: 2), |
| AsnLysIleSerTyrGlnSerSerSer- | (SEQ.ID.NO.: 3), |
| AsnLysIleSerTyrGlnSerSerSerThr- | (SEQ.ID.NO.: 4), |
| AsnLysIleSerTyrGlnSerSerSerThrGlu- | (SEQ.ID.NO.: 5), |
| AlaAsnLysIleSerTyrGlnSerSerSerThrGlu- | (SEQ.ID.NO.: 6), |
| Ac-AlaAsnLysIleSerTyrGlnSerSerSerThr- | (SEQ.ID.NO.: 7), |
| Ac-AlaAsnLysIleSerTyrGlnSerSerSerThrLeu- | (SEQ.ID.NO.: 8), |
| Ac-AlaAsnLysAlaSerTyrGlnSerAlaSerThrLeu- | (SEQ.ID.NO.: 9), |
| Ac-AlaAsnLysAlaSerTyrGlnSerAlaSerLeu- | (SEQ.ID.NO.: 10), |
| Ac-AlaAsnLysAlaSerTyrGlnSerSerSerLeu- | (SEQ.ID.NO.: 11), |
| Ac-AlaAsnLysAlaSerTyrGlnSerSerLeu- | (SEQ.ID.NO.: 12), |
| Ac-SerTyrGlnSerSerSerLeu- | (SEQ.ID.NO.: 13), |
| Ac-hArgTyrGlnSerSerSerLeu- | (SEQ.ID.NO.: 14). |
| Ac-LysTyrGlnSerSerSerLeu- | (SEQ.ID.NO.: 15), or |
| Ac-LysTyrGlnSerSerNle- | (SEQ.ID.NO.: 16); |
| ii) | |
| 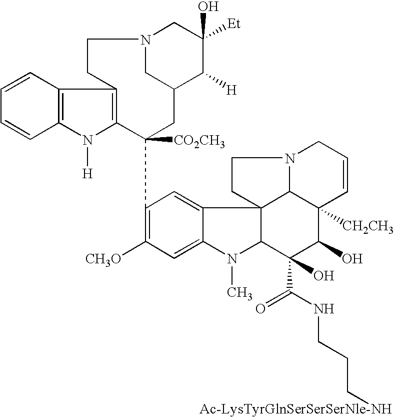 | (SEQ.ID.NO.: 17), or |
| 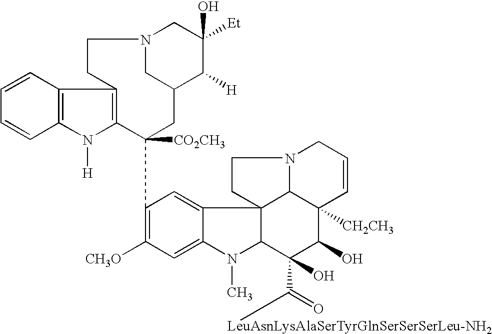 | (SEQ.ID.NO.: 18); |
| iii) | |
| 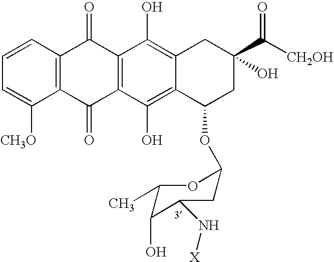 | |
| wherein X is: | |
| 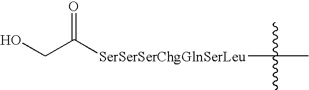 | (SEQ.ID.NO.: 19), |
| 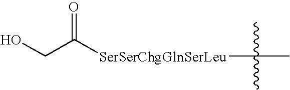 | (SEQ.ID.NO.: 20), or |
| 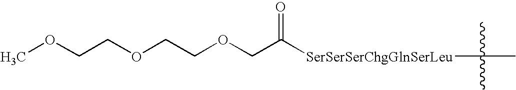 | (SEQ.ID.NO.:21); |
| iv) | |
| 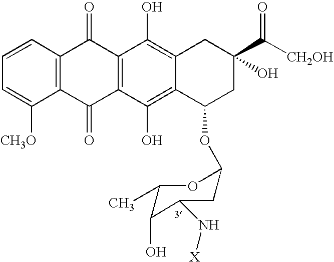 | |
| wherein X is: | |
| 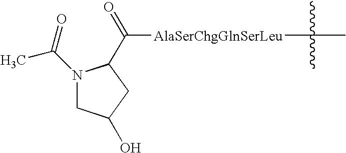 | (SEQ.ID.NO.: 22), |
| 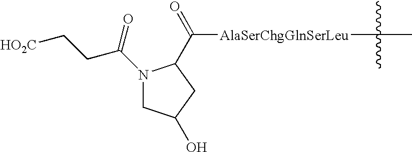 | (SEQ.ID.NO.: 23), |
| 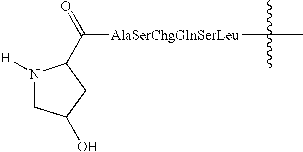 | (SEQ.ID.NO.: 24), |
| 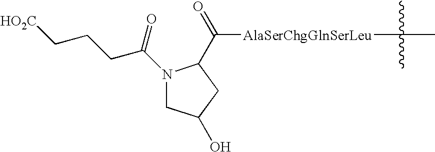 | (SEQ.ID.NO.: 25), |
| 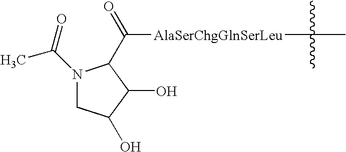 | (SEQ.ID.NO.: 26), |
| 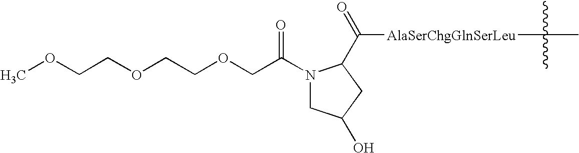 | (SEQ.ID.NO.: 27), or |
| 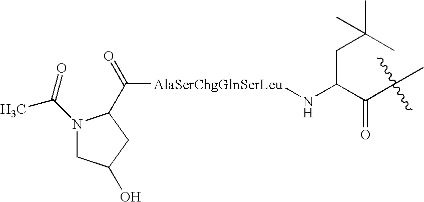 | (SEQ.ID.NO.: 28); |
| v) | |
| 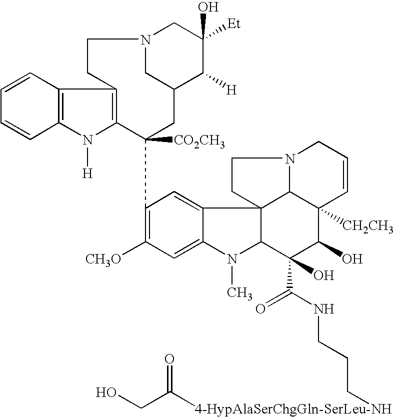 | (SEQ.ID.NO.: 29), |
| 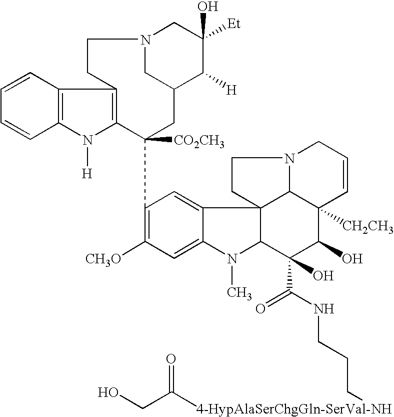 | (SEQ.ID.NO.: 30), or |
| 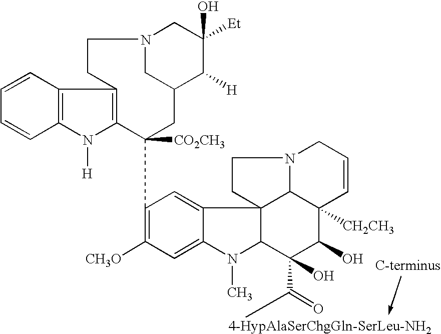 | (SEQ.ID.NO.: 31); |
| vi) | |
| 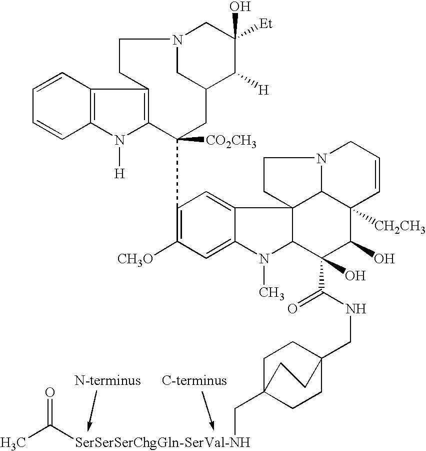 | (SEQ.ID.NO.: 32), |
| 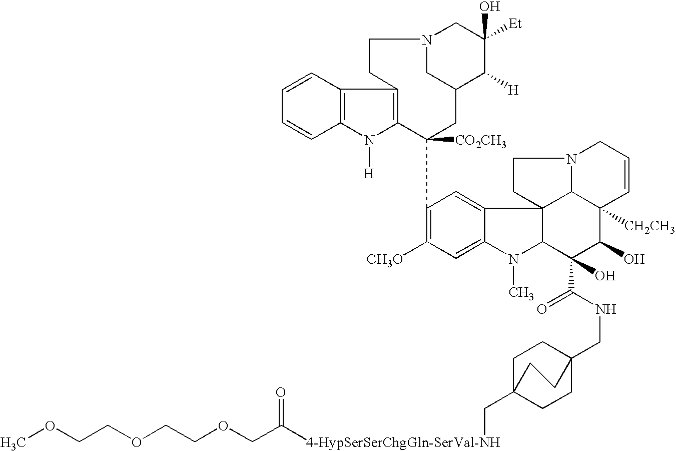 | (SEQ.ID.NO.: 33), |
| 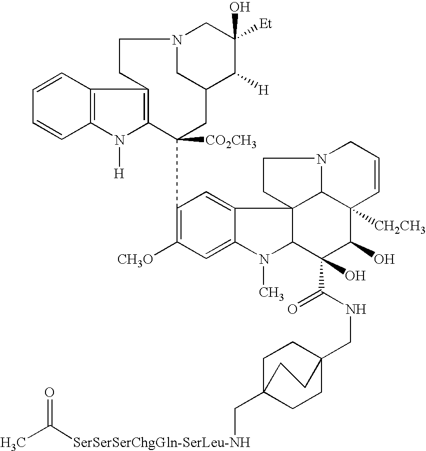 | (SEQ.ID.NO.: 34), or |
| 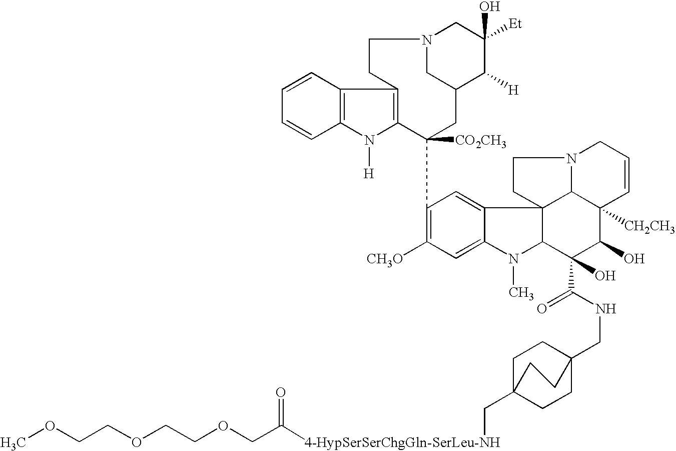 | (SEQ.ID.NO.: 35); |
| vii) | |
| 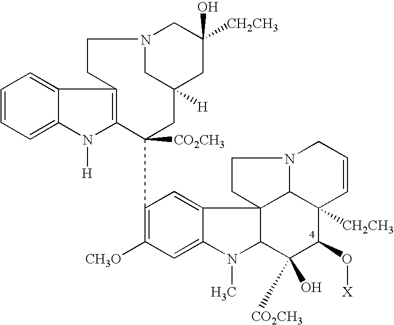 | |
| wherein X is | |
| 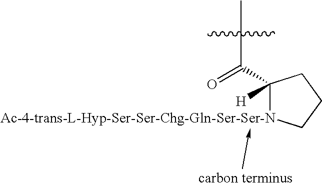 | (SEQ.ID.NO.: 36), |
| 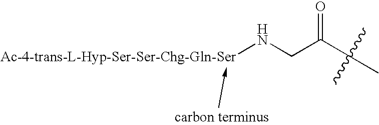 | (SEQ.ID.NO.: 37), |
| 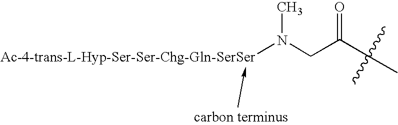 | (SEQ.ID.NO.: 38), |
| 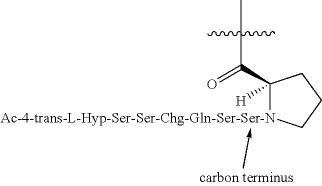 | (SEQ.ID.NO.: 39), |
| 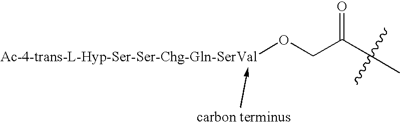 | (SEQ.ID.NO.: 40), |
| 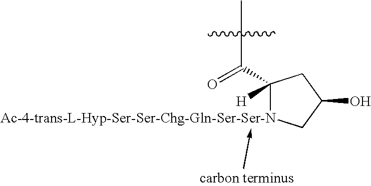 | (SEQ.ID.NO.: 41), |
| 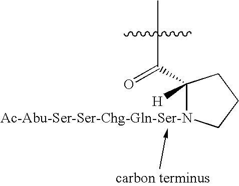 | (SEQ.ID.NO.: 42), |
| 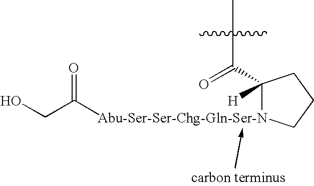 | (SEQ.ID.NO.: 43), |
| 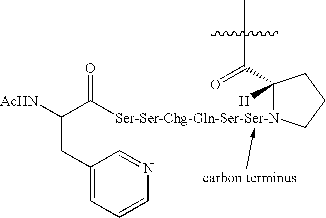 | (SEQ.ID.NO.: 44), |
| 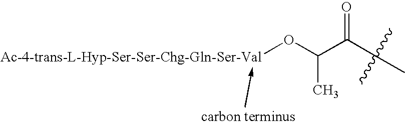 | (SEQ.ID.NO.: 45), or |
| 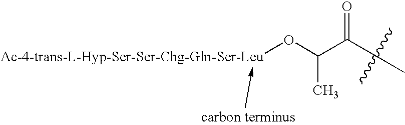 | (SEQ.ID.NO.: 46); |
| or the pharmaceutically acceptable salt or optical isomer thereof. | |

8. The method according to claim 7 wherein the PSA conjugate is: 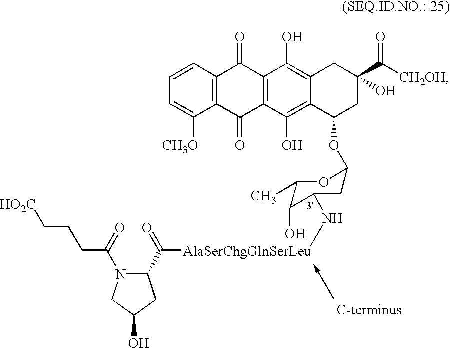
or a pharmaceutically acceptable salt thereof.

9. The method according to claim 1 wherein the tachykinin receptor antagonist is a neurokinin-1 (NK-1) receptor antagonist.

10. The method according to claim 9 wherein the neurokinin-1 (NK-1) receptor antagonist is selected from
a) a compound of formula (IX): 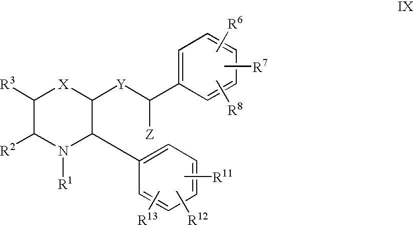
or a pharmaceutically acceptable salt thereof, wherein:
R¹ is selected from the group consisting of:
(1) hydrogen;
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo, wherein halo is fluoro, chloro, bromo or iodo,
(h) —NR⁹R¹⁰, wherein R⁹ and R¹⁰ are independently selected from:
(i) hydrogen,
(ii) C₁₋₆ alkyl,
(iii) hydroxy-C₁₋₆ alkyl, and
(iv) phenyl,
(i) —NR⁹COR¹⁰,
(j) —NR⁹CO₂R¹⁰,
(k) —CONR⁹R¹⁰,
(l) —COR⁹,
(m) —CO₂R⁹,
(n) heterocycle, wherein the heterocycle is selected from the group consisting of:
(A) benzimidazolyl,
(B) benzofuranyl,
(C) benzothiophenyl,
(D) benzoxazolyl,
(E) furanyl,
(F) imidazolyl,
(G) indolyl,
(H) isooxazolyl,
(I) isothiazolyl,
(J) oxadiazolyl,
(K) oxazolyl,
(L) pyrazinyl,
(M) pyrazolyl,
(N) pyridyl,
(O) pyrimidyl,
(P) pyrrolyl,
(Q) quinolyl,
(R) tetrazolyl,
(S) thiadiazolyl,
(T) thiazolyl,
(U) thienyl,
(V) triazolyl,
(W) azetidinyl,
(X) 1,4-dioxanyl,
(Y) hexahydroazepinyl,
(Z) piperazinyl,
(AA) piperidinyl,
(AB) pyrrolidinyl,
(AC) tetrahydrofuranyl, and
(AD) tetrahydrothienyl,
and wherein the heterocycle is unsubstituted or substituted with one or more substituent(s) selected from:
(i) C₁₋₆ alkyl, unsubstituted or substituted with halo, —CF₃, —OCH₃, or phenyl,
(ii) C₁₋₆ alkoxy,
(iii) oxo,
(iv) hydroxy,
(v) thioxo,
(vi) —SR⁹,
(vii) halo,
(viii) cyano,
(ix) phenyl,
(x) trifluoromethyl,
(xi) —(CH₂)ₘ—NR⁹R¹⁰, wherein m is 0, 1 or 2,
(xii) —NR⁹COR¹⁰,
(xiii) —CONR⁹R¹⁰,
(xiv) —CO₂R⁹, and
(xv) —(CH₂)ₘ—OR⁹;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹,
(j) —CO₂R⁹,
(k) heterocycle;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰,
(j) —NR⁹COR¹⁰,
(k) —NR⁹CO₂R¹⁰,
(l) —CONR⁹R¹⁰,
(m) —CO₂NR⁹R¹⁰,
(n) —COR⁹,
(o) —CO₂R⁹;
R² and R³ are independently selected from the group consisting of:
(1) hydrogen,
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰,
(i) —NR⁹COR¹⁰,
(j) —NR⁹CO₂R¹⁰,
(k) —CONR⁹R¹⁰,
(l) —COR⁹, and
(m) —CO₂R⁹;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹, and
(j) —CO₂R⁹;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰,
(j) —NR⁹COR¹⁰,
(k) —NR⁹CO₂R¹⁰,
(l) —CONR⁹R¹⁰,
(m) —CO₂NR⁹R¹⁰,
(n) —COR⁹, and
(o) —CO₂R⁹;
and the groups R¹ and R² may be joined together to form a heterocyclic ring selected from the group consisting of:
(a) pyrrolidinyl,
(b) piperidinyl,
(c) pyrrolyl,
(d) pyridinyl,
(e) imidazolyl,
(f) oxazolyl, and
(g) thiazolyl,
and wherein the heterocyclic ring is unsubstituted or substituted with one or more substituent(s) selected from:
(i) C₁₋₆alkyl,
(ii) oxo,
(iii) C₁₋₆alkoxy,
(iv) —NR⁹R¹⁰,
(v) halo, and
(vi) trifluoromethyl;
and the groups R² and R³ may be joined together to form a carbocyclic ring selected from the group consisting of:
(a) cyclopentyl,
(b) cyclohexyl,
(c) phenyl,
and wherein the carbocyclic ring is unsubstituted or substituted with one or more substituents selected from:
(i) C₁₋₆alkyl,
(ii) C₁₋₆alkoxy,
(iii) —NR⁹R¹⁰,
(iv) halo, and
(v) trifluoromethyl;
and the groups R² and R³ may be joined together to form a heterocyclic ring selected from the group consisting of:
(a) pyrrolidinyl,
(b) piperidinyl,
(c) pyrrolyl,
(d) pyridinyl,
(e) imidazolyl,
(f) furanyl,
(g) oxazolyl,
(h) thienyl, and
(i) thiazolyl,
and wherein the heterocyclic ring is unsubstituted or substituted with one or more substituent(s) selected from:
(i) C₁₋₆alkyl,
(ii) oxo,
(iii) C₁₋₆alkoxy,
(iv) —NR⁹R¹⁰,
(v) halo, and
(vi) trifluoromethyl;
R⁶, R⁷ and R⁸ are independently selected from the group consisting of:
(1) hydrogen;
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰,
(i) —NR⁹COR¹⁰,
(j) —NR⁹CO₂R¹⁰,
(k) —CONR⁹R¹⁰,
(l) —COR⁹, and
(m) —CO₂R⁹;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹, and
(j) —CO₂R⁹;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰,
(j) —NR⁹COR¹⁰,
(k) —NR⁹CO₂R¹⁰,
(l) —CONR⁹R¹⁰,
(m) —CO₂NR⁹R¹⁰,
(n) —COR⁹,
(o) —CO₂R⁹;
(6) halo,
(7) —CN,
(8) —CF₃,
(9) —NO₂,
(10) —SR¹⁴, wherein R¹⁴ is hydrogen or C₁₋₅alkyl,
(11) —SOR¹⁴,
(12) —SO₂R¹⁴,
(13) NR⁹COR¹⁰,
(14) CONR⁹COR¹⁰,
(15) NR⁹R¹⁰,
(16) NR⁹CO₂R¹⁰,
(17) hydroxy,
(18) C₁₋₆alkoxy,
(19) COR⁹,
(20) CO₂R⁹,
(21) 2-pyridyl,
(22) 3-pyridyl,
(23) 4-pyridyl,
(24) 5-tetrazolyl,
(25) 2-oxazolyl, and
(26) 2-thiazolyl;
R¹¹, R¹² and R¹³ are independently selected from the definitions of R⁶, R⁷ and R⁸;
X is selected from the group consisting of:
(1) —O—,
(2) —S—,
(3) —SO—, and
(4) —SO₂—;
Y is selected from the group consisting of:
(1) a single bond,
(2) —O—,
(3) —S—,
(4) —CO—,
(5) —CH₂—,
(6) —CHR¹⁵—, and
(7) —CR¹⁵R¹⁶—, wherein R¹⁵ and R¹⁶ are independently selected from the group consisting of:
(a) C₁₋₆ alkyl, unsubstituted or substituted with one or more of the substituents selected from:
(i) hydroxy,
(ii) oxo,
(iii) C₁₋₆ alkoxy,
(iv) phenyl-C₁₋₃ alkoxy,
(v) phenyl,
(vi) —CN,
(vii) halo,
(viii) —NR⁹R¹⁰,
(ix) —NR⁹COR¹⁰,
(x) —NR⁹CO₂R¹⁰,
(xi) —CONR⁹R¹⁰,
(xii) —COR⁹, and
(xiii) —CO₂R⁹;
(b) phenyl, unsubstituted or substituted with one or more of the substituent(s) selected from:
(i) hydroxy,
(ii) C₁₋₆ alkoxy,
(iii) C₁₋₆ alkyl,
(iv) C₂₋₅ alkenyl,
(v) halo,
(vi) —CN,
(vii) —NO₂,
(viii) —CF₃,
(ix) —(CH₂)ₘ—NR⁹R¹⁰,
(x) —NR⁹COR¹⁰,
(xi) —NR⁹CO₂R¹⁰,
(xii) —CONR⁹R¹⁰,
(xiii) —CO₂NR⁹R¹⁰,
(xiv) —COR⁹, and
(xv) —CO₂R⁹; and
Z is C₁₋₆ alkyl;
b) a compound of formula (X): 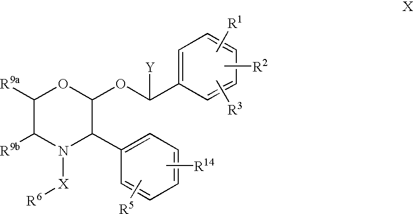
or a pharmaceutically acceptable salt or prodrug thereof, wherein
R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;
R² is hydrogen, halogen, C₁₋₆alkyl, C₁₋₆alkoxy substituted by C₁₋₄alkoxy or CF₃;
R³ is hydrogen, halogen or CF₃;
R⁴ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;
R⁵ is hydrogen, halogen, C₁₋₆alkyl, C₁₋₆alkoxy substituted by C₁₋₄alkoxy or CF₃;
R⁶ is a 5-membered or 6-membered heterocyclic ring containing 2 or 3 nitrogen atoms optionally substituted by ═O, ═S or a C₁₋₄alkyl group, and optionally substituted by a group of the formula ZNR⁷R⁸ where
Z is C₁₋₆alkylene or C₃₋₆cycloalkylene;
R⁷ is hydrogen, C₁₋₄alkyl, C₃₋₇cycloalkyl or C₃₋₇cycloalkylC₁₋₄ alkyl, or C₂₋₄alkyl substituted by C₁₋₄alkoxy or hydroxyl;
R⁸ is hydrogen, C₁₋₄alkyl, C₃₋₇cycloalkyl or C₃₋₇cycloalkylC₁₋₄ alkyl, or C₂₋₄alkyl substituted by one or two substituents selected from C₁₋₄alkoxy, hydroxyl or a 4, 5 or 6 membered heteroaliphatic ring containing one or two heteroatoms selected from N, O and S;
or R⁷, R⁸ and the nitrogen atom to which they are attached form a heteroaliphatic ring of 4 to 7 ring atoms, optionally substituted by a hydroxy group, and optionally containing a double bond, which ring may optionally contain an oxygen or sulphur ring atom, a group S(O) or S(O)₂ or a second nitrogen atom which will be part of a NH or NR^{C} moiety where R^{c} is C₁₋₄alkyl optionally substituted by hydroxy or C₁₋₄alkoxy;
or R⁷, R⁸ and the nitrogen atom to which they are attached form a non-aromatic azabicyclic ring system of 6 to 12 ring atoms; or Z, R⁷ and the nitrogen atom to which they are attached form a heteroaliphatic ring of 4 to 7 ring atoms which may optionally contain an oxygen ring atom;
R^{9a} and R^{9b} are each independently hydrogen or C₁₋₄alkyl, or R^{9a} and R^{9b} are joined so, together with the carbon atoms to which they are attached, there is formed a C₅₋₇ ring;
X is an alkylene chain of 1 to 4 carbon atoms optionally substituted by oxo; and
Y is a C₁₋₄alkyl group optionally substituted by a hydroxyl group; with the proviso that if Y is C₁₋₄alkyl, R⁶ is substituted at least by a group of formula ZNR⁷R⁸ as defined above;
d) a compound of formula (XI): 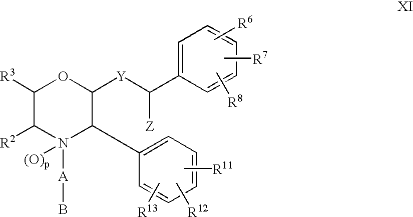
or a pharmaceutically acceptable salt thereof, wherein:
R² and R³ are independently selected from the group consisting of:
(1) hydrogen,
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are independently selected from:
(i) hydrogen,
(ii) C₁₋₆ alkyl,
(iii) hydroxy-C₁₋₆ alkyl, and
(iv) phenyl,
(i) —NR⁹COR¹⁰,
(j) —NR⁹CO₂R¹⁰,
(k) —CONR⁹R¹⁰,
(l) —COR⁹, and
(m) —CO₂R⁹;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹, and
(j) —CO₂R⁹;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰,
(j) —NR⁹COR¹⁰,
(k) —NR⁹CO₂R¹⁰,
(l) —CONR⁹R¹⁰,
(m) —CO₂NR⁹R¹⁰,
(n) —COR⁹, and
(o) —CO₂R⁹;
and, alternatively, the groups R² and R³ are joined together to form a carbocyclic ring selected from the group consisting of:
(a) cyclopentyl,
(b) cyclohexyl,
(c) phenyl,
and wherein the carbocyclic ring is unsubstituted or substituted with one or more substituents selected from:
(i) C₁₋₆alkyl,
(ii) C₁₋₆alkoxy,
(iii) —NR⁹R¹⁰,
(iv) halo, and
v) trifluoromethyl;
and, alternatively, the groups R² and R³ are joined together to form a heterocyclic ring selected from the group consisting of:
(a) pyrrolidinyl,
(b) piperidinyl,
(c) pyrrolyl,
(d) pyridinyl,
(e) imidazolyl,
(f) furanyl,
(g) oxazolyl,
(h) thienyl, and
(i) thiazolyl,
and wherein the heterocyclic ring is unsubstituted or substituted with one or more substituent(s) selected from:
(i) C₁₋₆alkyl,
(ii) oxo,
(iii) C₁₋₆alkoxy,
(iv) —NR⁹R¹⁰,
(v) halo, and
(vi) trifluoromethyl;
R⁶, R⁷ and R⁸ are independently selected from the group consisting of:
(1) hydrogen;
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰,
(i) —NR⁹COR¹⁰,
(j) —NR⁹CO₂R¹⁰,
(k) —CONR⁹R¹⁰,
(l) —COR⁹, and
(m) —CO₂R⁹;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹, and
(j) —CO₂R⁹;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰,
(j) —NR⁹COR¹⁰,
(k) —NR⁹CO₂R¹⁰,
(l) —CONR⁹R¹⁰,
(m) —CO₂NR⁹R¹⁰,
(n) —COR⁹, and
(o) —CO₂R⁹;
(6) halo,
(7) —CN,
(8) —CF₃,
(9) —NO₂,
(10) —SR¹⁴, wherein R¹⁴ is hydrogen or C₁₋₅alkyl,
(11) —SOR¹⁴,
(12) —SO₂R¹⁴,
(13) NR⁹COR¹⁰,
(14) CONR⁹COR¹⁰,
(15) NR⁹R¹⁰,
(16) NR⁹CO₂R¹⁰,
(17) hydroxy,
(18) C₁₋₆alkoxy,
(19) COR⁹,
(20) CO₂R⁹,
(21) 2-pyridyl,
(22) 3-pyridyl,
(23) 4-pyridyl,
(24) 5-tetrazolyl,
(25) 2-oxazolyl, and
(26) 2-thiazolyl;
R¹¹, R¹² and R¹³ are independently selected from the definitions of R⁶, R⁷ and R⁸, or —OX;
A is selected from the group consisting of:
(1) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰,
(i) —NR⁹COR¹⁰,
(j) —NR⁹CO₂R¹⁰,
(k) —CONR⁹R¹⁰,
(l) —COR⁹, and
(m) —CO₂R⁹;
(2) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰,
(i) —COR⁹, and
(j) —CO₂R⁹; and
(3) C₂₋₆ alkynyl;
B is a heterocycle, wherein the heterocycle is selected from the group consisting of: 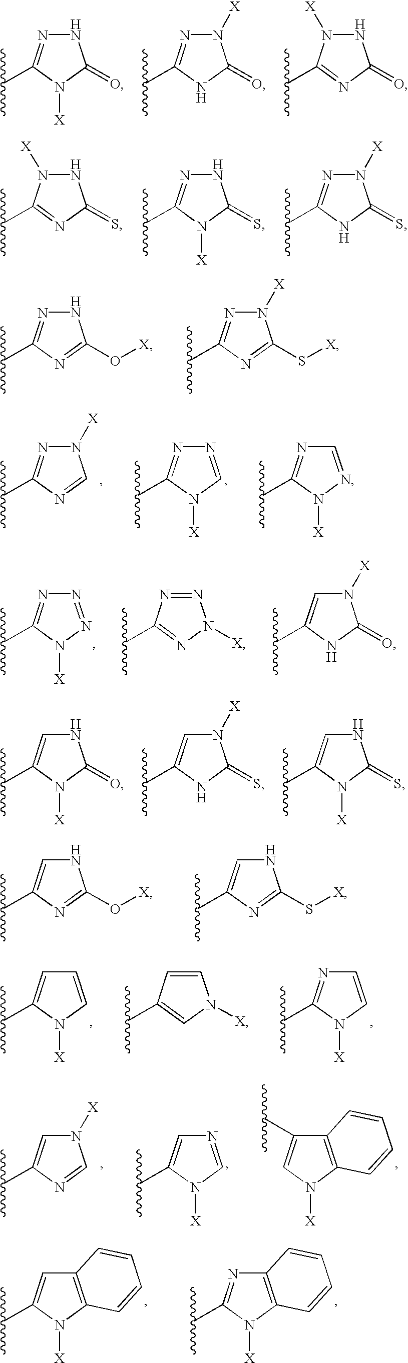
and wherein the heterocycle is substituted in addition to —X with one or more substituent(s) selected from:
(i) hydrogen;
(ii) C₁₋₆ alkyl, unsubstituted or substituted with halo, —CF₃, —OCH₃, or phenyl,
(iii) C₁₋₆ alkoxy,
(iv) oxo,
(v) hydroxy,
(vi) thioxo,
(vii) —SR⁹,
(viii) halo,
(ix) cyano,
(x) phenyl,
(xi) trifluoromethyl,
(xii) —(CH₂)ₘ—NR⁹R¹⁰, wherein m is 0, 1 or 2,
(xiii) —NR⁹COR¹⁰,
(xiv) —CONR⁹R¹⁰,
(xv) —CO₂R⁹, and
(xvi) —(CH₂)ₘ—OR⁹;
p is 0 or 1;
X is selected from:
(a) —PO(OH)O⁻.M⁺, wherein M⁺ is a pharmaceutically acceptable monovalent counterion,
(b) —PO(O⁻)₂.2M⁺,
(c) —PO(O⁻)₂.D²⁺, wherein D²⁺ is a pharmaceutically acceptable divalent counterion,
(d) —CH(R⁴)—PO(OH)O⁻.M⁺, wherein R⁴ is hydrogen or C₁₋₃ alkyl,
(e) —CH(R⁴)—PO(O⁻)₂.2M⁺,
(f) —CH(R⁴)—PP(O⁻)₂.D²⁺,
(g) —SO₃⁻.M+,
(h) —CH(R⁴)—SO₃⁻.M⁺,
(i) —CO—CH₂CH₂—CO₂⁻. M⁺,
(j) —CH(CH₃)—O—CO—R⁵, wherein R⁵ is selected from the group consisting of: 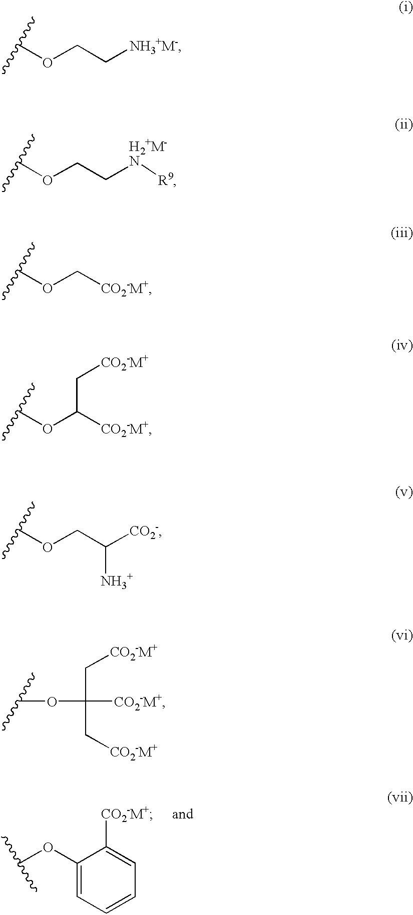
(k) hydrogen, with the proviso that if p is 0 and none of R¹¹, R¹² or R¹³ are —OX, then X is other than hydrogen;
Y is selected from the group consisting of:
(1) a single bond,
(2) —O—,
(3) —S—,
(4) —CO—,
(5) —CH₂—,
(6) —CHR¹⁵—, and
(7) —CR¹⁵R¹⁶—, wherein R¹⁵ and R¹⁶ are independently selected from the group consisting of:
(a) C₁₋₆ alkyl, unsubstituted or substituted with one or more of the substituents selected from:
(i) hydroxy,
(ii) oxo,
(iii) C₁₋₆ alkoxy,
(iv) phenyl-C₁₋₃ alkoxy,
(v) phenyl,
(vi) —CN,
(vii) halo,
(viii) —NR⁹R¹⁰,
(ix) —NR⁹COR¹¹,
(x) —NR⁹CO₂R¹⁰,
(xi) —CONR⁹R¹⁰,
(xii) —COR⁹, and
(xiii) —CO₂R⁹;
(b) phenyl, unsubstituted or substituted with one or more of the substituent(s) selected from:
(i) hydroxy,
(ii) C₁₋₆ alkoxy,
(iii) C₁₋₆ alkyl,
(iv) C₂₋₅ alkenyl,
(v) halo,
(vi) —CN,
(vii) —NO₂,
(viii) —CF₃,
(ix) —(CH₂)ₘ—NR⁹R¹⁰,
(x) —NR⁹COR¹⁰,
(xi) —NR⁹CO₂R¹⁰,
(xii) —CONR⁹R¹⁰,
(xiii) —CO₂NR⁹R¹⁰,
(xiv) —COR⁹, and
(xv) —CO₂R⁹;
Z is selected from:
(1) hydrogen,
(2) C₁₋₆ alkyl, and
(3) hydroxy, with the proviso that if Y is —O—, Z is other than hydroxy, or if Y is —CHR¹⁵—, then Z and R¹⁵ are optionally joined together to form a double bond.
e) a compound of formula (XII): 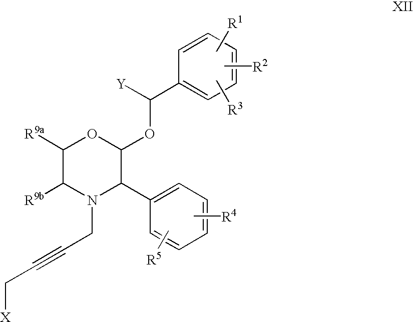
wherein:
X is a group of the formula NR⁶R⁷ or a C- or N-linked imidazolyl ring;
Y is hydrogen or C₁₋₄alkyl optionally substituted by a hydroxy group;
R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} each independently represent hydrogen or C₁₋₄alkyl;
R² is hydrogen, halogen, C₁₋₆alkyl, C₁₋₆alkoxy substituted by C₁₋₄ alkoxy or CF₃;
R³ is hydrogen, halogen or CF₃;
R⁴ is hydrogen, halogen, C₁₋₆alkyl, C₁₋₆alkoxy, hydroxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} are as previously defined;
R⁵ is hydrogen, halogen, C₁₋₆alkyl, C₁₋₆alkoxy substituted by C₁₋₄alkoxy or CF₃;
R⁶ is hydrogen, C₁₋₆alkyl, C₃₋₇cycloalkyl, C₃₋₇cycloalkylC₁₋₄alkyl, phenyl, or C₂₋₄alkyl substituted by C₁₋₄alkoxy or hydroxy;
R⁷ is hydrogen, C₁₋₆alkyl, C₃₋₇cycloalkyl, C₃₋₇cycloalkylC₁₋₄alkyl, phenyl, or C₂₋₄alkyl substituted by one or two substituents selected from C₁₋₄alkoxy, hydroxy or a 4, 5 or 6 membered heteroaliphatic ring containing one or two heteroatoms selected from N, O and S;
or R⁶ and R⁷, together with the nitrogen atom to which they are attached, form a saturated or partially saturated heterocyclic ring of 4 to 7 ring atoms, which ring may optionally contain in the ring one oxygen or sulphur atom or a group selected from NR⁸, S(O) or S(O)₂ and which ring may be optionally substituted by one or two groups selected from hydroxyC₁₋₄alkyl, C₁₋₄alkoxyC₁₋₄alkyl, oxo, COR^{a} or CO₂R^{a} where R^{a} is as previously defined;
or R⁶ and R⁷ together with the nitrogen atom to which they are attached, form a non-aromatic azabicyclic ring system of 6 to 12 ring atoms;
R⁸ is hydrogen, C₁₋₄alkyl, hydroxyC₁₋₄alkyl or C₁₋₄alkoxyC₁₋₄alkyl; and
R^{9a} and R^{9b} are each independently hydrogen or C₁₋₄alkyl, or R^{9a} and R^{9b} are joined so, together with the carbon atoms to which they are attached, there is formed a C₅₋₇ ring;
and pharmaceutically acceptable salts thereof.
f) a compound of formula (XIII): 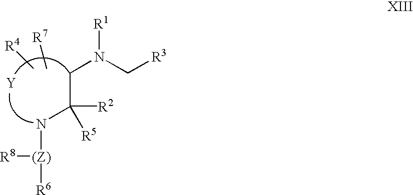
or a pharmaceutically acceptable salt thereof, wherein
Y is (CH₂)ₙ wherein n is an integer from 1 to 4, and wherein any one of the carbon-carbon single bonds in said (CH₂)ₙ may optionally be replaced by a carbon-carbon double bond, and wherein any one of the carbon atoms of said (CH₂)ₙ may optionally be substituted with R⁴, and wherein any one of the carbon atoms of said (CH₂)ₙ may optionally be substituted with R⁷;
Z is (CH₂)ₘ wherein m is an integer from 0 to 6, and wherein any one of the carbon-carbon single bonds of (CH₂)ₘ may optionally be replaced by a carbon-carbon double bond or a carbon-carbon triple bond, and any one of the carbon atoms of said (CH₂)ₘ may optionally be substituted with R⁸;
R¹ is hydrogen or C₁₋₈alkyl optionally substituted with hydroxy, C₁₋₄alkoxy or fluoro;
R² is a radical selected from hydrogen, C₁₋₆ straight or branched alkyl, C₃₋₇cycloalkyl wherein one of the CH₂ groups in said cycloalkyl may optionally be replaced by NH, oxygen or sulphur; aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; phenyl-C₂₋₆ alkyl, benzhydryl and benzyl, wherein each of said aryl and heteroaryl groups and the phenyl moieties of said benzyl, phenyl —C₂₋₆alkyl and benzhydryl may optionally be substituted with one or more substituents independently selected from halo, nitro, C₁₋₆alkyl, C₁₋₆ alkoxy, trifluoromethyl, amino, C₁₋₆alkylamino, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO, C₁₋₆alkyl-CO-C₁₋₆alkyl-, di-C₁₋₆alkylamino, —CONH—C₁₋₆alkyl, C₁₋₆alkyl-CO—NH—C₁₋₆alkyl, —NHCOH and —NHCO—C₁₋₆alkyl; and wherein one of the phenyl moieties of said benzhydryl may optionally be replaced by naphthyl, thienyl, furyl or pyridyl;
R⁵ is hydrogen, phenyl or C₁₋₆alkyl;
or R² and R⁵ together with the carbon to which they are attached, form a saturated ring having from 3 to 7 carbon atoms wherein one of the CH₂ groups in said ring may optionally be replaced by oxygen, NH or sulfur;
R³ is aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; and cycloalkyl having 3 to 7 carbon atoms wherein one of the (CH₂) groups in said cycloalkyl may optionally be replaced by NH, oxygen or sulphur;
wherein each of said aryl and heteroaryl groups may optionally be substituted with one or more substituents, and said C₃₋₇cycloalkyl may optionally be substituted with one or two substituents, each of said substituents being independently selected from halo, nitro, C₁₋₆alkyl, C₁₋₆alkoxy, trifluoromethyl, amino, C₁₋₆ alkylamino, —CO—NH—C₁₋₆alkyl, C₁₋₆alkyl-CO—NH—C₁₋₆alkyl, —NHCOH and —NH CO—C₁₋₆alkyl;
R⁴ and R⁷ are each independently selected from hydroxy, halogen, halo, amino, oxo, cyano, methylene, hydroxymethyl, halomethyl, C₁₋₆alkylamino, di-C₁₋₆alkylamino, C₁₋₆alkoxy, C₁₋₆alkyl-O—CO, C₁₋₆alkyl-O—CO—C₁₋₆alkyl, C₁₋₆ alkyl-CO—O, C₁₋₆alkyl-CO—C₁₋₆alkyl-O—, C₁₋₆alkyl-CO—, C₁₋₆alkyl-CO—C₁₋₆alkyl, and the radicals set forth in the definition of R²;
R⁶ is —NHCOR⁹, —NHCH₂R⁹, SO₂R⁸ or one of the radicals set forth in any of the definitions of R², R⁴ and R⁷;
R⁸ is oximino (═NOH) or one of the radicals set forth in any of the definitions of R², R⁴ and R⁷;
R⁹ is C₁₋₆alkyl, hydrogen, phenyl or phenylC₁₋₆alkyl;
with the proviso that (a) when m is 0, R⁸ is absent, (b) when R⁴, R⁶, R⁷ or R⁸ is as defined in R², it cannot form together with the carbon to which it is attached, a ring with R⁵, and (c) when R⁴ and R⁷ are attached to the same carbon atom, then either each of R⁴ and R⁷ is independently selected from hydrogen, fluoro and C₁₋₆alkyl, or R⁴ and R⁷, together with the carbon to which they are attached, for a C₃₋₆ saturated carbocyclic ring that forms a spiro compound with the nitrogen-containing ring to which they are attached;
g) a compound of formula (XIV): 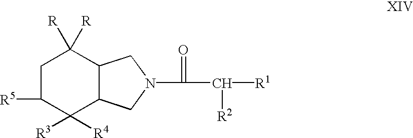
or a pharmaceutically acceptable salt thereof, wherein
radicals R are phenyl radicals optionally 2- or 3-substituted by a halogen atom or a methyl radical;
R¹ is optionally substituted phenyl, cyclohexadienyl, naphthyl, indenyl or optionally substituted heterocycle;
R² is H, halogen, OH, alkyl, aminoalkyl, alkylaminoalkyl, dialkylaminoalkyl, alkyloxy, alkylthio, acyloxy, carboxy, optionally substituted alkyloxycarbonyl, benzyloxycarbonyl, amino or acylamino;
R³ is optionally 2-substituted phenyl;
R⁴ is OH or fluorine when R⁵ is H;
or R⁴ and R⁵ are OH;
or R⁴ and R⁵ together form a bond;
h) a compound of formula (XV): 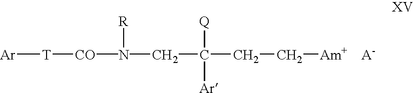
wherein
Ar represents an optionally substituted mono-, di- or tricyclic aromatic or heteroaromatic group;
T represents a bond, a hydroxymethylene group, a C₁₋₄ alkoxymethylene group or a C₁₋₅alkylene group;
Ar′ represents a phenyl group which is unsubstituted or substituted by one or more substituents selected from halogen, preferably chlorine or fluorine, trifluoromethyl, C₁₋₄alkoxy, C₁₋₄alkyl where the said substituents may be the same or different; a thienyl group; a benzothienyl group; a naphthyl group; or an indolyl group;
R represents hydrogen, C₁₋₄alkyl, —C₁₋₄alkoxyC₁₋₄alkyl, or —C₂₋₄ alkanoyloxyC₂₋₄alkyl;
Q represents hydrogen; or
Q and R together form a 1,2-ethylene, 1,3-propylene or 1,4-butylene group;
Am⁺ represents the radical 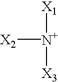
in which X₁, X₂ and X₃, together with the nitrogen atom to which they are attached, form an azabicyclic or azatricyclic ring system optionally substituted by a phenyl or benzyl group; and
A⁻ represents a pharmaceutically acceptable anion;
i) a compound of formula (XVI): 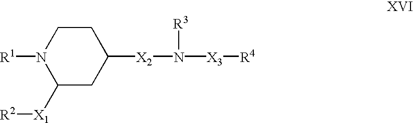
or a pharmaceutically acceptable salt thereof, wherein
R¹ represents an optionally substituted aralkyl, aryloxyalkyl, heteroaralkyl, aroyl, heteroaroyl, cycloalkylcarbonyl, aralka noyl, heteroarylalkanoyl, aralkoxycarbonyl or arylcarbamoyl group or the acyl group of an (-amino acid optionally N-substituted by a lower alkanoyl or carbamoyl-lower alkanoyl group;
R² represents cycloalkyl or an optionally substituted aryl or heteroaryl group;
R³ represents hydrogen, alkyl, carbamoyl or an alkanoyl or alkenoyl group optionally substituted by carboxy or esterified or amidated carboxy;
R⁴ represents an optionally substituted aryl group or an optionally partially saturated heteroaryl group;
X₁ represents methylene, ethylene, a bond, an optionally ketalised carbonyl group or an optionally etherified hydroxymethylene group;
X₂ represents alkylene, carbonyl or a bond; and
X₃ represents carbonyl, oxo-lower alkyl, oxo(aza)-lower alkyl, or an alkyl group optionally substituted by phenyl, hydroxymethyl, optionally esterified or amidated carboxy, or (in other than the (-position) hydroxy.
j) a compound of formula (XVII): 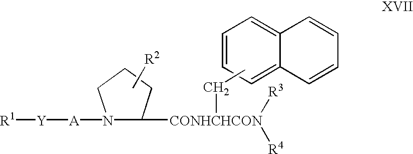
or a pharmaceutically acceptable salt thereof, wherein R¹ is aryl, or a group of the formula: 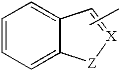
or a pharmaceutically acceptable salt thereof, wherein
X is CH or N;
Z is O or N—R⁵, in which R⁵ is hydrogen or lower alkyl;
R² is hydroxy or lower alkoxy;
R³ is hydrogen or optionally substituted lower alkyl;
R⁴ is optionally substituted ar(lower)alkyl;
A is carbonyl or sulfonyl; and
Y is a bond or lower alkenylene;
k) a compound of the formula (XVIII): 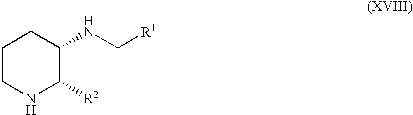
or a pharmaceutically acceptable salt thereof, wherein
R¹⁰ is aryl selected from indanyl, phenyl and naphthyl; heteroaryl selected from thienyl, furyl, pyridyl and quinolyl; and cycloalkyl having 3 to 7 carbon atoms, wherein one of said carbon atoms may optionally be replaced by nitrogen, oxygen or sulfur; wherein each of said aryl and heteroaryl groups may optionally be substituted with one or more substituents, and said C₃₋₇cycloalkyl may optionally be substituted with one or two substituents, said substituents being independently selected from chloro, fluoro, bromo, iodo, nitro, C₁₋₁₀alkyl optionally substituted with from one to three fluoro groups, C₁₋₁₀alkoxy optionally substituted with from one to three fluoro groups, amino, C₁₋₁₀alkyl-S—, C₁₋₁₀alkyl-S(O)—, C₁₋₁₀alkyl-SO₂—, phenyl, phenoxy, C₁₋₁₀alkyl-SO₂NH—, C₁₋₁₀alkyl-SO₂NH—C₁₋₁₀alkyl-, C₁₋₁₀alkylamino-diC₁₋₁₀alkyl-, cyano, hydroxy, cycloalkoxy having 3 to 7 carbon atoms, C₁₋₆alkylamino, C₁₋₆ dialkylamino, HC(O)NH— and C₁₋₁₀alkyl-C(O)NH—; and
R² is thienyl, benzhydryl, naphthyl or phenyl optionally substituted with from one to three substituents independently selected from chloro, bromo, fluoro, iodo, cycloalkoxy having 3 to 7 carbon atoms, C₁₋₁₀alkyl optionally substituted with from one to three fluoro groups and C₁₋₁₀alkoxy optionally substituted with from one to three fluoro groups;
l) a compound of formula (XIX): 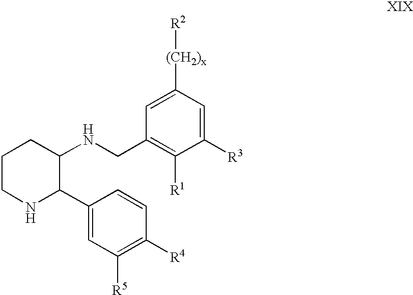
wherein
R¹ is a C₁₋₄alkoxy group;
R² is 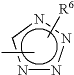
R³ is a hydrogen or halogen atom;
R⁴ and R⁵ may each independently represent a hydrogen or halogen atom, or a C₁₋₄ alkyl, C₁₋₄ alkoxy or trifluoromethyl group;
R⁶ is a hydrogen atom, a C₁₋₄ alkyl, (CH₂)ₘ cyclopropyl, —S(O)ₙ C₁₋₄ alkyl, phenyl, NR⁷R⁸, CH₂C(O)CF₃ or trifluoromethyl group;
R⁷ and R⁸ may each independently represent a hydrogen atom, or a C₁₋₄ alkyl or acyl group;
x represents zero or 1;
n represents zero, 1 or 2;
m represents zero or 1;
and pharmaceutically acceptable salts and solvates thereof.
m) a compound of formula (XX): 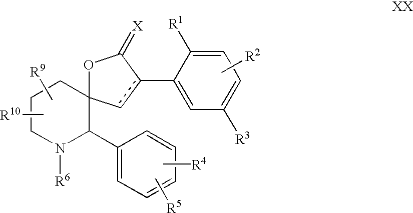
wherein
R¹, R², R³, R⁴, R⁵, R⁶, R⁹, R¹⁰, and X are as defined therein;
n) a compound of formula (XXI): 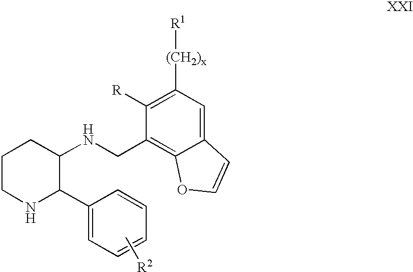
wherein
R represents a hydrogen atom or a C₁₋₄ alkoxy group;
R¹ is selected from phenyl, optionally substituted by a group —(CH₂)ₙCONR³R⁴ or S(O)ₘR³; or a 5- or 6-membered aromatic heterocycle containing 1, 2, 3 or 4 heteroatoms selected from oxygen, nitrogen, or sulphur, optionally substituted by a C₁₋₄ alkyl, trifluoromethyl or cyano group or a group —(CH₂)ₙCONR³R⁴;
R² represents a hydrogen or halogen atom;
R³ and R⁴ independently represent hydrogen or C₁₋₄ alkyl;
n represents zero, 1 or 2;
m represents zero, 1 or 2;
z represents zero or 1;
o) a compound of formula (XXII) 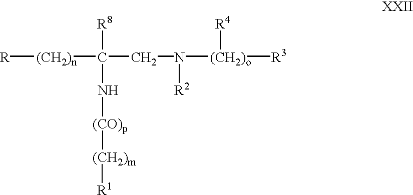
a pharmaceutically acceptable salt thereof, wherein
m is zero, 1, 2 or 3;
n is zero or 1;
o is zero, 1 or 2;
p is zero or 1;
R is phenyl, 2- or 3-indolyl, 2- or 3-indolinyl, benzothienyl, benzofuranyl, or naphthyl;
which R groups may be substituted with one or two halo, C₁₋₃alkoxy, trifluoromethyl, C₁₋₄alkyl, phenyl-C₁₋₃alkoxy, or C₁₋₄alkanoyl groups;
R¹ is trityl, phenyl, diphenylmethyl, phenoxy, phenylthio, piperazinyl, piperidinyl, pyrrolidinyl, morpholinyl, indolinyl, indolyl, benzothienyl, hexamethyleneiminyl, benzofuranyl, tetrahydropyridinyl, quinolinyl, isoquinolinyl, reduced quinolinyl, reduced isoquinolinyl, phenyl-(C₁₋₄alkyl)-, phenyl-(C₁₋₄alkoxy)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-(C₁₋₃alkyl)-, C₁₋₄alkyl, or —NH—CH₂—R;
any one of which R¹ groups may be substituted with halo, C₁₋₄alkyl, C₁₋₄alkoxy, trifluoromethyl, amino, C₁₋₄alkylamino, di(C₁₋₄alkyl)amino, or C₂₋₄ alkanoylamino;
or any one of which R¹ groups may be substituted with phenyl, piperazinyl, C₃₋₈cycloalkyl, benzyl, C₁₋₄alkyl, piperidinyl, pyridinyl, pyrimidinyl, C₂₋₆ alkanoylamino, pyrrolidinyl, C₂₋₆alkanoyl, or C₁₋₄alkoxycarbonyl;
any one of which groups may be substituted with halo, C₁₋₄alkyl, C₁₋₄ alkoxy, trifluoromethyl, amino, C₁₋₄alkylamino, di(C₁₋₄alkyl)amino, or C₂₋₄ alkanoylamino;
or R¹ is amino, a leaving group, hydrogen, C₁₋₄alkylamino, or di(C₁₋₄alkyl)amino;
R⁵ is pyridyl, anilino-(C₁₋₃alkyl)-, or anilinocarbonyl;
R² is hydrogen, C₁₋₄alkyl, C₁₋₄alkylsulfonyl, carboxy-(C₁₋₃alkyl)-, C₁₋₃alkoxycarbonyl-(C₁₋₃alkyl)-, or —CO—R⁶;
R⁶ is hydrogen, C₁₋₄alkyl, C₁₋₃haloalkyl, phenyl, C₁₋₃alkoxy, C₁₋₃hydroxyalkyl, amino, C₁₋₄alkylamino, di(C₁₋₄alkyl)amino, or —(CH₂)_{q}—R⁷;
q is zero to 3;
R⁷ is carboxy, C₁₋₄alkoxycarbonyl, C₁₋₄alkylcarbonyloxy, amino, C₁₋₄alkylamino, di(C₁₋₄alkyl)amino, C₁₋₆alkoxycarbonylamino, or phenoxy, phenylthio, piperazinyl, piperidinyl, pyrrolidinyl, morpholinyl, indolinyl, indolyl, benzothienyl, benzofuranyl, quinolinyl, phenyl-(C₁₋₄alkyl)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-C₁₋₃alkyl;
any one of which aryl or heterocyclic R⁷ groups may be substituted with halo, trifluoromethyl, C₁₋₄alkoxy, C₁₋₄alkyl, amino, C₁₋₄alkylamino, di(C₁₋₄alkyl) amino, or C₂₋₄alkanoylamino;
or any one of which R⁷ groups may be substituted with phenyl, piperazinyl, C₃₋₈cycloalkyl, benzyl, piperidinyl, pyridinyl, pyrimidinyl, pyrrolidinyl, C₂₋₆alkanoyl, or C₁₋₄alkoxycarbonyl;
any of which groups may be substituted with halo, trifluoromethyl, amino, C₁₋₄alkoxy, C₁₋₄alkyl, C₁₋₄alkylamino, di(C₁₋₄alkyl)amino, or C₂₋₄alkanoylamino;
R⁸ is hydrogen or C₁₋₆alkyl;
R³ is phenyl, phenyl-(C₁₋₆alkyl)-, C₃₋₈cycloalkyl, C₅₋₈cycloalkenyl, C₁₋₈ alkyl, naphthyl, C₂₋₈alkenyl, or hydrogen;
any one or which groups except hydrogen may be substituted with one or two halo, C₁₋₃alkoxy, C₁₋₃alkylthio, nitro, trifluoromethyl, or C₁₋₃alkyl groups; and
R⁴ is hydrogen or C₁₋₃alkyl;
with the proviso that if R¹ is hydrogen or halo, R³ is phenyl, phenyl-(C₁₋₆alkyl)-, C₃₋₈cycloalkyl, C₅₋₈cycloalkenyl, or naphthyl.

11. The method according to claim 10 wherein the neurokinin-1 (NK-1) receptor antagonist is selected from:
4-(3-(1,2,4-triazolo)methyl)-2(S)-(3,5-bis(trifluoro-methyl)benzyloxy)-3(S)-phenylmorpholine;
4-(3-(1,2,4-triazolo)methyl)-2(S)-(3,5-bis(trifluoro-methyl)benzyloxy)-3(R)-phenylmorpholine;
4-(3-(5-oxo-1H,4H-1,2,4-triazolo)methyl)-2(S)-(3,5-bis(trifluoromethyl)benzyloxy)-3(S)-phenyl-morpholine; and
2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methylmorpholine;
2-(R)-2-(R)-(1-(R)-(3,5-bis(trifluoromethyl)phenyl)ethoxy)-4-(5-(dimethylamino) methyl-1,2,3-triazol-4-yl)methyl-3-(S)-phenylmorpholine;
(1-(R)-(3,5-bis(trifluoromethyl)phenyl)ethoxy)-4-((dimethylamino-methyl)-1,2,3-triazol-4-yl)methyl)-3-(S)-(4-fluorophenyl)morpholine;
(1-(R)-(3,5-bis(trifluoromethyl)phenyl)ethoxy)-3-(S)-(4-fluoro)-phenyl-4-(3-(1-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(4-morpholinobut-2-yn-yl)morpholine;
2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;
4-(4-azetidinylbut-2-yn-yl)-2-(R)-(1-(R)-(3,5-bis(trifluoromethyl)phenyl) ethoxy)-3-(S)-(4-fluorophenyl)morpholine;
2-(R)-(1-(R)-(3,5-bis(trifluoromethyl)phenyl)ethoxy)-3-(S)-(4-fluorophenyl)-4-(4-imidazolylbut-2-yn-yl)morpholine;
2-(R)-(1-(R)-(3,5-bis(trifluoromethyl)phenyl)ethoxy)-3-(S)-(4-fluorophenyl)-4-(4-(N-methylpiperazinyl)but-2-yn-yl)morpholine;
4-(4-bis(2-methoxyethyl)aminobut-2-yn-yl)-2-(R)-(1-(R)-(3,5-bis(trifluoromethyl) phenyl)ethoxy)-3-(S)-(4-fluorophenyl)morpholine;
2-(R)-(1-(R)-(3,5-bis(trifluoromethyl)phenyl)ethoxy)-3-(S)-(4-fluorophenyl)-4-(4-pyrrolidinobut-2-yn-yl)morpholine;
3-(S)-(4-fluorophenyl)-2-(R)-(1-(R)-(3-fluoro-5-(trifluoromethyl)phenyl)ethoxy)-4-(4-morpholinobut-2-yn-yl)morpholine;
3-(S)-(4-fluorophenyl)-4-(4-morpholinobut-2-yn-yl)-2-(R)-(1-(R)-(3-(trifluoromethyl) phenyl)ethoxy)morpholine;
4-(4-azetidinylbut-2-yn-yl)-3-(S)-(4-fluorophenyl)-2-(R)-(1-(R)-(3-(trifluoromethyl) phenyl)ethoxy)morpholine;
2-(R)-(1-(R)-(3,5-bis(trifluoromethyl)phenyl)ethoxy)-4-(4-(N-(2-methoxyethyl)-N-methyl)aminobut-2-yn-yl)-3-(S)-phenylmorpholine;
2-(R)-(1-(R)-(3,5-bis(trifluoromethyl)phenyl)ethoxy)-4-(4-(N-cyclopropyl-N-(2-methoxyethyl)amino)but-2-yn-yl)-3-(S)-phenylmorpholine;
2-(R)-(1-(R)-(3,5-bis(trifluoromethyl)phenyl)ethoxy)-4-(4-(N-isopropyl-N-(2-methoxyethyl)amino)but-2-yn-yl)-3-(S)-phenylmorpholine;
4-(4-(N,N-dimethylamino)but-2-yn-yl)-3-(S)-(4-fluorophenyl)-2-(R)-(1-(S)-(3-fluoro-5-(trifluoromethyl)phenyl-2-hydroxyethoxy)morpholine;
4-(4-azetidinylbut-2-yn-yl)-3-(S)-(4-fluorophenyl)-2-(R)-(1-(S)-(3-fluoro-5-(trifluoromethyl)phenyl)-2-hydroxyethoxy)morpholine;
2-(R)-(1-(S)-(3,5-bis(trifluoromethyl)phenyl)-2-hydroxyethoxy)-4-(4-(N,N-dimethylamino)but-2-yn-yl)-3-(S)-(4-fluorophenyl)morpholine;
4-(4-azetidinylbut-2-yn-yl)-2-(R)-(1-(S)-(3,5-bis(trifluoromethyl)phenyl-2-hydroxyethoxy)-3-(S)-(4-fluorophenyl)morpholine;
4-(4-N-bis(2-methoxy)ethyl-N-methylamino)but-2-yn-yl)-2-(R)-(1-(R)-(3,5-bis(trifluoromethyl)phenyl)ethoxy)-3-(S)-(4-fluorophenyl)morpholine;
2-(R)-(1-(R)-(3,5-bis(trifluoromethyl)phenyl)ethoxy)-3-(S)-(4-fluorophenyl)-4-(4-(2-(S)-(methoxymethyl)pyrrolidino)but-2-yn-yl)morpholine;
4-(4-(7-azabicyclo[2.2.1]heptano)but-2-yn-yl)-2-(R)-(1-(R)-(3,5-bis(trifluoromethyl) phenyl)ethoxy)-3-(S)-(4-fluorophenyl)morpholine;
2-(R)-(1-(R)-(3,5-bis(trifluoromethyl)phenyl)ethoxy)-4-(4-diisopropylaminobut-2-yn-yl)-3-(S)-(4-fluorophenyl)morpholine;
2-(R)-(1-(R)-(3-fluoro-5-(trifluoromethyl)phenyl)ethoxy)-4-(4-(2-(S)-(methoxymethyl) pyrrolidino)but-2-yn-yl)-3-(S)-phenylmorpholine;
2-(R)-(1-(R)-(3,5-bis(trifluoromethyl)phenyl)ethoxy)-3-(S)-(4-fluorophenyl)-4-(4-(2-(S)-(hydroxymethyl)pyrrolidino)but-2-yn-yl)morpholine;
(2S,3S)-cis-3-(2-methoxybenzylamino)-2-phenylpiperidine;
(3aS,4S,7aS)-7,7-diphenyl-4-(2-methoxyphenyl)-2-[(2S)-(2-methoxyphenyl)propionyl]perhydroisoindol-4-ol;
(+) 1-[2-[3-(3,4-dichlorophenyl)-1-[(3-isopropoxyphenyl)acetyl]-3-piperidinyl]ethyl]-4-phenyl-1-azabicyclo[2,2,2]octane; (2R*,4S *)-2-benzyl-1-(3,5-dimethylbenzoyl)-N-(4-quinolinylmethyl)-4-piperidineamine;
(2S,3S)-3-(2-methoxy-5-trifluoromethoxybenzyl)-amino-2-phenylpiperidine;
[2-methoxy-5-(5-trifluoromethyl-tetrazol-1-yl)-benzyl]-(2S-phenyl-piperidin-3S-yl)-amine;
(3S,5R,6S)-3-[2-cyclopropoxy-5-(trifluoromethoxy)phenyl]-6-phenyl-1-oxa-7-aza-spiro[4.5]decane; (3R,5R,6S)-3-[2-cyclopropoxy-5-(trifluoromethoxy)phenyl]-6-phenyl-1-oxa-7-aza-spiro[4.5]decane;
[5-(5-methyl-tetrazol-1-yl)-benzofuran-7-ylmethyl]-(2S-phenyl-piperidin-3S-yl)-amine;
[N-(2-methoxybenzyl)acetylamino]-3-(1H-indol-3-yl)-2-[N-(2-(4-piperidin-1-yl)piperidin-1-yl)acetylamino]propane; 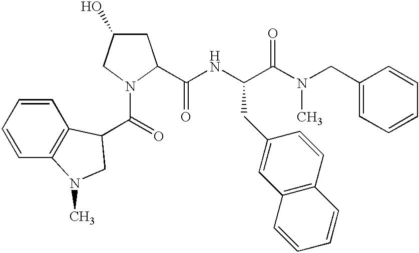
or a pharmaceutically acceptable salt thereof.

12. The method of claim 10, wherein the neurokinin-1 (NK-1) receptor antagonist is selected from:
(±)-(2R,3R,2S3S)-N-{[2-cyclopropoxy-5-(trifluoromethoxy)-phenyl]methyl}-2-phenylpiperidin-3-amine;
2-(S)-(3,5-bis(trifluoromethyl)benzyloxy)-3(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;
2-(S)-(3,5-bis(trifluoromethyl)benzyloxy)-4-(3-(5-oxo-1H,4H-1,2,4-triazolo) methyl)-3-(S)-phenyl-morpholine;
2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-4-(5-(N,N-dimethylamino) methyl-1,2,3-triazol-4-yl)methyl-3-(S)-phenylmorpholine;
2-(R)-(1-(R)-(3,5-bis(trifluoromethyl)phenyl)ethoxy)-4-(5-(N,N-dimethylamino) methyl-1,2,3-triazol-4-yl)methyl-3-(S)-(4-fluorophenyl)morpholine;
(3S,5R,6S)-3-[2-cyclopropoxy-5-(trifluoromethoxy)phenyl]-6-phenyl-1-oxa-7-aza-spiro[4.5]decane; (3R,R,6S)-3-[2-cyclopropoxy-5-(trifluoromethoxy)phenyl]-6-phenyl-1-oxa-7-aza-spiro[4.5]decane;
2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;
2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;
2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;
2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;
or a pharmaceutically acceptable salt thereof.

13. A pharmaceutical composition for achieving a therapeutic effect in a mammal in need thereof which comprises amounts of at least one tachykinin receptor antagonist and at least one PSA conjugate.

14. The pharmaceutical composition according to claim 13 comprising an amount of an tachykinin receptor antagonist and an amount of a PSA conjugate.

15. The pharmaceutical composition according to claim 13 wherein the therapeutic effect is selected from inhibition of cancerous tumor growth and the regression of cancerous tumors.

16. The composition according to claim 13 wherein the cancer is a cancer related to cells that express enzymatically active PSA.

17. The composition according to claim 16 wherein the cancer is prostate cancer.

18. A method of preparing a pharmaceutical composition for treatment of cancer in a mammal in need thereof which comprises mixing amounts of at least one tachykinin receptor antagonist and at least one PSA conjugate.

19. The method of preparing a pharmaceutical composition according to claim 18 comprising mixing an amount of a tachykinin receptor antagonist and an amount of an PSA conjugate.

20. A method of treating cancer in a mammal in need thereof which comprises administering to said mammal amounts of at least one tachykinin receptor antagonist and at least one PSA conjugate and applying to the mammal radiation therapy.

21. The method according to claim 20 wherein an amount of a tachykinin receptor antagonist and an amount of a PSA conjugate are administered simultaneously.

22. The method according to claim 20 wherein an amount of a tachykinin receptor antagonist and an amount of a PSA conjugate are administered consecutively.

23. A method for treating prostatic disease in a mammal in need thereof which comprises administering to said mammal amounts of at least one tachykinin receptor antagonist and at least one PSA conjugate.

24. The method according to claim 23 wherein the prostatic disease is selected from benign prostatic hyperplasia, prostatic intraepithelial meoplasia and prostate cancer.


SEQUENCE LISTING (46 sequences)

SEQ ID NO: 1 - protein, 7 aa, Artificial Sequence
  completely synthetic amino acid sequence
NKISYQS

SEQ ID NO: 2 - protein, 8 aa, Artificial Sequence
  completely synthetic amino acid sequence
NKISYQSS

SEQ ID NO: 3 - protein, 9 aa, Artificial Sequence
  completely synthetic amino acid sequence
NKISYQSSS

SEQ ID NO: 4 - protein, 10 aa, Artificial Sequence
  completely synthetic amino acid sequence
NKISYQSSST

SEQ ID NO: 5 - protein, 11 aa, Artificial Sequence
  completely synthetic amino acid sequence
NKISYQSSSTE

SEQ ID NO: 6 - protein, 12 aa, Artificial Sequence
  completely synthetic amino acid sequence
ANKISYQSSSTE

SEQ ID NO: 7 - protein, 11 aa, Artificial Sequence
  completely synthetic amino acid sequence
ANKISYQSSST

SEQ ID NO: 8 - protein, 12 aa, Artificial Sequence
  completely synthetic amino acid sequence
ANKISYQSSSTL

SEQ ID NO: 9 - protein, 12 aa, Artificial Sequence
  completely synthetic amino acid sequence
ANKASYQSASTL

SEQ ID NO: 10 - protein, 11 aa, Artificial Sequence
  completely synthetic amino acid sequence
ANKASYQSASL

SEQ ID NO: 11 - protein, 11 aa, Artificial Sequence
  completely synthetic amino acid sequence
ANKASYQSSSL

SEQ ID NO: 12 - protein, 10 aa, Artificial Sequence
  completely synthetic amino acid sequence
ANKASYQSSL

SEQ ID NO: 13 - protein, 7 aa, Artificial Sequence
  completely synthetic amino acid sequence
SYQSSSL

SEQ ID NO: 14 - protein, 7 aa, Artificial Sequence
  completely synthetic amino acid sequence
RYQSSSL

SEQ ID NO: 15 - protein, 7 aa, Artificial Sequence
  completely synthetic amino acid sequence
KYQSSSL

SEQ ID NO: 16 - protein, 6 aa, Artificial Sequence
  completely synthetic amino acid sequence
KYQSSL

SEQ ID NO: 17 - protein, 7 aa, Artificial Sequence
  completely synthetic amino acid sequence
KYQSSSL

SEQ ID NO: 18 - protein, 11 aa, Artificial Sequence
  completely synthetic amino acid sequence
LNKASYQSSSL

SEQ ID NO: 19 - protein, 7 aa, Artificial Sequence
  completely synthetic amino acid sequence
XSSXQSL

SEQ ID NO: 20 - protein, 6 aa, Artificial Sequence
  completely synthetic amino acid sequence
XSXQSL

SEQ ID NO: 21 - protein, 7 aa, Artificial Sequence
  completely synthetic amino acid sequence
XSSXQSL

SEQ ID NO: 22 - protein, 7 aa, Artificial Sequence
  completely synthetic amino acid sequence
XASXQSL

SEQ ID NO: 23 - protein, 7 aa, Artificial Sequence
  completely synthetic amino acid sequence
XASXQSL

SEQ ID NO: 24 - protein, 7 aa, Artificial Sequence
  completely synthetic amino acid sequence
PASXQSL

SEQ ID NO: 25 - protein, 7 aa, Artificial Sequence
  completely synthetic amino acid sequence
XASXQSL

SEQ ID NO: 26 - protein, 7 aa, Artificial Sequence
  completely synthetic amino acid sequence
XASXQSL

SEQ ID NO: 27 - protein, 7 aa, Artificial Sequence
  completely synthetic amino acid sequence
XASXQSL

SEQ ID NO: 28 - protein, 7 aa, Artificial Sequence
  completely synthetic amino acid sequence
XASXQSX

SEQ ID NO: 29 - protein, 7 aa, Artificial Sequence
  completely synthetic amino acid sequence
XASXQSL

SEQ ID NO: 30 - protein, 7 aa, Artificial Sequence
  completely synthetic amino acid sequence
XASXQSV

SEQ ID NO: 31 - protein, 7 aa, Artificial Sequence
  completely synthetic amino acid sequence
PASXQSL

SEQ ID NO: 32 - protein, 7 aa, Artificial Sequence
  completely synthetic amino acid sequence
SSSXQSV

SEQ ID NO: 33 - protein, 7 aa, Artificial Sequence
  completely synthetic amino acid sequence
XSSXQSV

SEQ ID NO: 34 - protein, 7 aa, Artificial Sequence
  completely synthetic amino acid sequence
SSSXQSL

SEQ ID NO: 35 - protein, 7 aa, Artificial Sequence
  completely synthetic amino acid sequence
XSSXQSL

SEQ ID NO: 36 - protein, 8 aa, Artificial Sequence
  completely synthetic amino acid sequence
XSSXQSSP

SEQ ID NO: 37 - protein, 7 aa, Artificial Sequence
  completely synthetic amino acid sequence
XSSXQSG

SEQ ID NO: 38 - protein, 8 aa, Artificial Sequence
  completely synthetic amino acid sequence
XSSXQSSG

SEQ ID NO: 39 - protein, 8 aa, Artificial Sequence
  completely synthetic amino acid sequence
XSSXQSSP

SEQ ID NO: 40 - protein, 7 aa, Artificial Sequence
  completely synthetic amino acid sequence
XSSXQSV

SEQ ID NO: 41 - protein, 8 aa, Artificial Sequence
  completely synthetic amino acid sequence
XSSXQSSP

SEQ ID NO: 42 - protein, 7 aa, Artificial Sequence
  completely synthetic amino acid sequence
XSSXQSP

SEQ ID NO: 43 - protein, 7 aa, Artificial Sequence
  completely synthetic amino acid sequence
XSSXQSP

SEQ ID NO: 44 - protein, 8 aa, Artificial Sequence
  completely synthetic amino acid sequence
XSSXQSSP

SEQ ID NO: 45 - protein, 7 aa, Artificial Sequence
  completely synthetic amino acid sequence
XSSXQSV

SEQ ID NO: 46 - protein, 7 aa, Artificial Sequence
  completely synthetic amino acid sequence
XSSXQSL